(19) United States
(12) Patent Application Publication
(10) Pub. No.: US 2010/0080784 A1
(43) Pub. Date: Apr. 1, 2010

(54) METHODS FOR TREATING CACHEXIA AND LYMPHOPENIA

(75) Inventor: Joanna Davies, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: TORREY PINES INSTITUTE FOR MOLECULAR STUDIES, San Diego, CA (US)

(21) Appl. No.: 12/557,373

(22) Filed: Sep. 10, 2009

Related U.S. Application Data
(60) Provisional application No. 61/096,706, filed on Sep. 12, 2008.

(51) Int. Cl. A61K 35/12; A61P 43/00; A61P 3/10; C12Q 1/06

(52) U.S. Cl. 424/93.71; 435/39

(57) ABSTRACT

Disappearance of a cell population, designated CD4⁺ CD44^{v.low}, has been shown to be associated with cachexia and lymphopenia, and those conditions can be treated or delayed by administering those cells to a patient. In addition, disclosed are assays for those cells for diagnosing or prognosticating cachexia and/or lymphopenia and the end of the honeymoon period in Type I diabetes. Furthermore, disclosed herein are methods related to the use of CD4+ CD44v.low cells in promoting insulin-secreting beta cell mass.

### CROSS-REFERENCE TO RELATED APPLICATIONS

This application claims the benefit of priority under 35 U.S.C. §119 to U.S. Provisional Patent Application No. 61/096,706, filed Sep. 12, 2008, and which is expressly incorporated herein by reference in its entirety.

### BACKGROUND OF THE INVENTION

1. Field of the Invention

Aspects of the present invention relate to cellular immunology, molecular biology and medicine. More specifically, some embodiments include methods that relate to the treatment, diagnosis, and prognosis of cachexia and/or lymphopenia.

2. Description of the Related Art

Cachexia (Donnelly and Walsh. Semin Oncol 1995; 22:67-72; Strawford and Hellerstein. Semin Oncol 1988; 25:76-81; Grounds M D. Biogerontology 2002; 3:19-24; Wallace and Schwartz. Int J Cardiol 2002; 85:15-21; Nair et al. J Clin Invest 1995; 95:2926-37) is the dramatic weight loss and muscle atrophy seen in patients with cancer (Donnelly and Walsh. Semin Oncol 1995; 22:67-72) and AIDS (Strawford and Hellerstein. Semin Oncol 1988; 25:76-81) as well as in aging individuals (Grounds M D. Biogerontology 2002; 3:19-24; Wallace and Schwartz. Int J Cardiol 2002; 85:15-21) and in certain autoimmune conditions, including Type I Diabetes (TID) (Nair et al. J Clin Invest 1995; 95:2926-37). TID is an autoimmune disorder caused by the immune-mediated destruction of insulin-secreting pancreatic beta cells, resulting in low insulin production and high blood glucose levels. (Castano and Eisenbarth. Annu Rev Immunol 1990; 8:647-79; Tisch and McDevitt. Cell 1996; 85:291-7). Diabetes can be controlled with daily insulin injections. However, in the long term, diabetes leads to a variety of complications including muscle atrophy and cachexia. (Nair et al. J Clin Invest 1995; 95:2926-37; Charlton and Nair. J Nutr 1998; 128:323S-7S; Vogiatzi et al. J Clin Endocrinol Metab 1997; 82:4083-7).

Accordingly, many investigators seek to design and develop new cachexia therapies so as to improve survival and the quality of life of patients suffering from cachexia.

The honeymoon period in Type I Diabetes (TID) is the transient partial remission seen primarily in children with new onset TID. Treatment is most effective in those patients with the highest residual β-cell function at the time of treatment, ie. during the honeymoon period. Therefore, identifying biomarkers that can accurately identify the honeymoon period is likely to be extremely important in identifying patients who are most likely to respond to treatments aimed at reversing TID. In addition, identifying strategies that delay the loss of the honeymoon period would also be highly significant.

### SUMMARY OF THE INVENTION

Disclosed herein are methods related method of treating, ameliorating, preventing, or delaying the onset of cachexia in a patient comprising administering isolated or purified CD4+ T cells to the patient. The cell sample can be obtained from a mammal. The mammal may or may not be the patient. Some embodiments involve isolating or purifiying CD4+ T cells from the cell sample and expanding the isolated or purified T cells. In some embodiments, the isolated or purified CD4+ T cells are CD4+ CD44^{v.low}. In other embodiments, the cell sample is blood sample. In further embodiments, the cachexia is associated with a disease selected from the group consisting of diabetes mellitus (e.g., Type I diabetes), cancer, and AIDS. In some embodiments, the T cells can be isolated using antibodies (e.g., anti-CD4 and/or anti-CD44 antibodies). In some embodiments, the T cells are expanded using growth factors and/or antibodies. In some embodiments, the T cells administered to the patient include from about 10⁸ to about 10¹¹ cells.

Additionally, disclosed herein are isolated T cell populations and pharmaceutical compositions comprising the T cell populations. In some embodiments, the population is characterized as CD4+ CD44^{v.low}.

Disclosed herein are methods related to inhibiting or reversing lymphopenia in a patient comprising administering isolated or purified CD4+ T cells to the patient. Some embodiments further involve obtaining a cell sample from a mammal, isolating or purifying CD4+ T cells from the cell sample, and expanding the isolated or purified T cells. In some embodiments, the isolated or purified CD4+ T cells are CD4+ CD44^{v.low}. In other embodiments, a therapy is further provided to the patient.

Additional embodiments related to methods of treating, ameliorating or preventing diabetes in a patient comprising CD4+ T cells to the patient. Some embodiments further involve obtaining a cell sample from a mammal, isolating or purifying CD4+ T cells from the cell sample, and expanding the isolated or purified T cells. In some embodiments, the isolated or purified CD4+ T cells are CD4+ CD44^{v.low}.

Further embodiments relate to methods for diagnosing cachexia in a patient, comprising identifying a patient at risk for cachexia, determining a level of CD430 CD44^{v.low} T cells in a biological sample from said patient, and assessing whether the amount of CD4+ CD44^{v.low} T cells is at a level which is lower than a predetermined level.

Other embodiments relate to methods for diagnosing the onset of a honeymoon period in a patient suffering from Type 1 diabetes, comprising identifying a patient with Type 1 diabetes prior to said honeymoon period, determining a level of CD4+ CD44^{v.low} T cells in a biological sample from said patient, and assessing whether the amount of CD4+ CD44^{v.low} T cells is at a level which is higher than a predetermined level.

Further embodiments relate to methods for diagnosing the loss of a honeymoon period in a patient suffering from Type 1 diabetes, comprising identifying a patient with Type 1 diabetes within said honeymoon period, determining a level of CD4+ CD44^{v.low} T cells in a biological sample from said patient, and assessing whether the amount of CD4+ CD44^{v.low} T cells is at a level which is lower than a predetermined level.

More embodiments relate to methods for monitoring the progress of a cachexia therapy in a patient comprising identifying a patient with cachexia, providing said subject a cachexia therapy, and determining a level of CD4+ CD44^{v.low} T cells in a biological sample in said patient, before a treatment with said cachexia therapy and during or after a period of said treatment.

Some embodiments relate to methods for determining the response to a cachexia therapy in a patient comprising identifying a patient with a cachexia, providing said patient a cachexia therapy, and determining a level of CD4+ CD44^{v.low} T cells in a biological sample in said patient, before a treatment with said cachexia therapy and during or after a period of said treatment.

Other embodiments relate to methods for promoting the responsiveness to a therapy for a disorder comprising identifying a patient with the disorder and administering isolated or purified CD4+ T cells to the patient. Some embodiments further involve obtaining a cell sample from a mammal, isolating or purifying CD4+ T cells from the cell sample, and expanding the isolated or purified T cells. In some embodiments, the isolated or purified CD4+ T cells are CD4+ CD44^{v.low}. Some embodiments involve providing to said patient said therapy for said disorder (e.g., cachexia and/or Type I diabetes).

Additional embodiments relate to methods of identifying a patient likely to be responsive to a therapy for a disorder comprising identifying a patient with said disorder, determining a level of CD4+ CD44^{v.low} T cells in a biological sample from said patient, and assessing whether the amount of CD4+ CD44^{v.low} T cells is at a level which is greater than a predetermined level. The disorder can be, for example, cachexia or Type 1 diabetes.

Some embodiments relate to methods for delaying the onset of the honeymoon period in Type 1 diabetes, comprising administering isolated or purified CD4+ T cells to the patient. Some embodiments further involve obtaining a cell sample from a mammal, isolating or purifying CD4+ T cells from the cell sample, and expanding the isolated or purified T cells. In some embodiments, the isolated or purified CD4+ T cells are CD4+ CD44^{v.low}.

Other embodiments relate to methods for delaying the loss of the honeymoon period in Type 1 diabetes, comprising administering isolated or purified CD4+ T cells to the patient. Some embodiments further involve obtaining a cell sample from a mammal, isolating or purifying CD4+ T cells from the cell sample, and expanding the isolated or purified T cells. In some embodiments, the isolated or purified CD4+ T cells are CD4+ CD44^{v.low}.

Additional embodiments relate to methods of treating, ameliorating or preventing diabetes in a patient comprising isolating or purifying pancreatic islets from the patient and/or other donor, growing the islets in culture in the presence of CD4+ T cells (e.g., CD4+ CD44^{v.low} cells), and transplanting the islets into the patient.

### BRIEF DESCRIPTION OF THE DRAWINGS

FIG. 1. NOD mice lose weight post-diabetes onset. NOD female mice were monitored for the development of diabetes and wasting (n=22). Panel a shows the percentage of mice that were diabetic (closed square) and wasting (open square) over a fifteen-week period. Panel b shows the relationship between age of diabetes onset and the age at onset of wasting. The closed circle indicates an individual diabetic mouse while the closed circle x2 indicates two mice that became diabetic and wasting on the same day (n=14). Using Spearman Rank Correlation the linear correlation between the age at onset of diabetes and the age at onset of wasting was found to be highly significant (p<0.0001) with a correlation coefficient of 0.9877 and a 95% confidence interval of 0.9591-0.9963. The data are pooled from two experiments.

FIG. 2. Weight loss in diabetic NOD mice is not associated with a reduction in food and water intake. Diabetic (closed circle, n=11) and non-diabetic (open circle, n=9) age matched female NOD mice were monitored for wasting (a), food intake (b) and water intake (c). Data show the mean±SEM for % of pre-diabetic body wt., food intake and water intake for diabetic mice post-diabetes onset (closed circle). Measurements are taken at the same time for age matched non-diabetic mice (open circle) for direct comparison.

FIG. 3. Cachexia in NOD mice is associated with apoptosis. Onset of wasting was monitored in female NOD mice from the age of 10 through 18 weeks. Skeletal muscle from five wasting diabetic mice and nine non-wasting diabetic mice were analyzed for evidence of apoptosis. The electrophoretic pattern of 2 representative samples of DNA from wasting mice (lanes 2 and 3, 24% and 20% weight loss respectively) and a single representative example of DNA from a non-wasting mouse (lane 4) is shown. The DNA ladder molecular weight markers and a positive control for apoptotic laddering are shown in lanes 1 and 5, respectively. The electrophoretic data is presented as a composite of lanes from the same gel. Using Fisher's Exact Test the data shows a significant correlation between the presence of DNA laddering in mice that are wasting and the lack of DNA fragmentation in mice that are not wasting (p=0.0005). The data are pooled from two experiments.

FIG. 4. Significant skeletal muscle protein loss is associated with wasting and not diabetes without wasting. Soluble protein extract was isolated from the gastrocnemius muscle of mice that were either, diabetic and wasting (D+W, n=6), or diabetic but not wasting (D+NW, n=7), or not diabetic and not wasting (ND+NW, n=7). Total soluble protein in each muscle was determined and the mean±SEM was compared between groups. Data are pooled from 2 independent experiments. The level of statistical significance is indicated as * for p=0.05-0.01, *** for p=0.0009-0.0001.

FIG. 5. Muscle atrophy in diabetic mice is associated with a significant increase in ubiquitin conjugation. The extent of ubiquitin conjugation of protein in the gastrocnemius muscle of mice that were either, diabetic and wasting (D+W, n=6), or diabetic and non-wasting (D+NW, n=7), or non-diabetic and non-wasting (ND+NW, n=7) was determined. Panel a; lanes 1 and 2 contain samples from two D+W mice, lanes 3-5 contain samples from three D+NW mice, and lanes 6-8 contain samples from three ND+NW mice. An equivalent amount of protein was loaded into each lane. The relative amount of ubiquitinated protein in muscle from each mouse was determined by measuring the number of pixels from 64 kDa-250 kDa for each lane. Panel b shows the mean±SEM for the total pixel number per lane between 64-250 kDa calculated for each group. Data are pooled from 2 independent experiments. The level of statistical significance is ** for p=0.009-0.001, *** for p=0.0009-0.0001.

FIG. 6. The E3 ligase MuRF1 is significantly upregulated at the onset of wasting, while upregulation of MAFbx requires both diabetes and wasting. The relative expression of MuRF1 and MAFbx in skeletal muscle from mice that were either, diabetic and wasting (D+W, n=6), or diabetic but not wasting (D+NW, n=7), or neither diabetic nor wasting (ND+NW, n=7) was determined by semi-quantitative RT-PCR. 62 -actin (400 bp) was used as an internal control. The expression of MuRF1 (panel a) and MAFbx (panel b) appear as single bands of 573 by and 845 by respectively. The relative expression of MuRF1 and MAFbx between groups was compared by determining the ratio of either MuRF1 or MAFbx to β-actin for each sample, and then comparing that ratio between groups. Panels a and b show representative image of expression of either MuRF1, or MAFbx (top band) and β-actin (lower band) of mice with D+W (lane 1), D+NW (lane 2), and ND+NW (lane 3). The mean+SEM of the ratio of MuRF1 to β-actin (panel c), or MAFbx to β-actin (panel d) is shown for each group. The level of statistical significance is * for p=0.05-0.01, ** for p=0.009-0.001.

FIG. 7. CD4⁺CD44^{v.low} cells are deficient in cachexia. The FACS profiles show the density of expression of CD44 on CD4⁺ splenocytes from representative examples of non-diabetic and non-cachexic (ND/NC, panels a and b), and diabetic and cachexic mice (D/C, panel c). The mean number of CD4 cells (±SEM) that express CD44 at either, a very low (CD44^{v.low}) density (panel d), or, at a low (CD44^{low}), or, high (CD44^{high}) density (panel e), in non-diabetic and non-cachexic (closed bars, ND/NC, n=5), diabetic and non-cachexic (hatched bars, D/NC, n=5) and diabetic and cachexic (open bars, D/C, n=5) mice is shown. The mean±SEM of the total number of CD4⁺ cells in each group is also shown in panel e. The level of statistical significance is indicated as * for p=0.05-0.01 and ** for p=0.009-0.001. The data are representative of three experiments.

FIG. 8. The phenotype of CD4⁺ CD44^{v.low} cells. Splenocytes from ten-week old NOD female mice were labeled with CD4- and CD44-specific antibodies in combination with antibodies specific for one of CD3 (n=8), CD25 (n=12), CD45RB (n=8), CD62L (n=8) or CD38 (n=8). Data shown in panel a are representative of the co-expression of CD44 and CD4 on NOD splenocytes. Data shown in panels b-f are representative of the co-expression of CD44 and either CD3 (panel b), CD25 (panel c), CD45RB (panel d), CD62L (panel e) or CD38 (panel f) on CD4⁻ cells in four separate experiments. The horizontal bars are set based on the isotype control profiles. CD4⁻ CD44^{v.low} cells are to the left of the vertical bars in each panel.

FIG. 9. Onset of diabetes and wasting in NOD mice. A group of NOD female mice (n=15) were monitored for the development of diabetes and wasting. Panel a shows the incidence of diabetes onset with age. Panel b shows body weight increase and loss (as a percentage of body weight at 10 weeks of age), for each of 7 diabetic mice. Percentage body weight change is shown from the time of diabetes onset to the time taken to lose 20% original body weight. The data are representative of two experiments.

FIG. 10. CD4⁺ CD44^{v.low} cells decreases the incidence of wasting, but not diabetes. The incidence of wasting (panel a) and diabetes (panel b) was compared in female NOD mice injected at 10 weeks of age with 2.5×10⁵ sorted CD4⁺ CD44^{v.low} cells (n=10, o) isolated from spleens of 11 week old pre-diabetic NOD female donors, or with no cells (n=12, ν). Body weight (c) and body weight as a percentage of weight of each mouse at 10 weeks of age (d), shows the severity of wasting in each group. Data in panels c and d are shown as mean±SEM. The data are representative of two experiments.

FIG. 11. CD4⁺ CD44^{v.low} cells significantly delay the onset of wasting when infused into diabetic NOD mice. Female NOD mice were monitored for the onset of diabetes. Within one week after diabetes was diagnosed, diabetic mice were infused with either 5×10⁵ sorted CD4⁺ CD44^{v.low} cells isolated from pre-diabetic NOD mice (n=16, ν), or, an equal number of CD4⁺ cells depleted of CD44^{v.low} cells (n=20, λ), also isolated from pre-diabetic NOD mice, or, with no cells (n=18, σ). The incidence and time of onset of wasting is shown for each group and compared using the Logrank Mantel Cox test. p=0.02 for untreated versus CD4⁺ CD44^{v.low} cell treatment groups.

FIG. 12. The effect of CD4⁺ CD44^{v.low} cells on insulin-secreting (β cells. The insulin positive area in the pancreas was compared in female NOD mice injected at 9 weeks of age with 2.5×10⁵ CD4⁺ CD44^{v.low} cells (treated, n=9), or with no cells (untreated, n=9). All mice were sacrificed at 15 weeks post-cell infusion and the amount of insulin in the pancreas, measured in pixels, is shown in panels a, d and g, as mean±SD for each group. The representative sections of pancreas shown are stained for the presence of insulin (brown coloration). Panel a shows the insulin area in diabetic untreated mice (7 out of 9 mice were diabetic and all diabetic mice were wasting) compared to diabetic treated mice (8 out of 9 mice were diabetic and 4 of the diabetic mice were also wasting). Panels b and c are representative sections of pancreata from diabetic untreated mice (b, diabetic and wasting), and diabetic treated (c, diabetic and wasting) mice. Panel d compares the insulin area in pancreata from treated diabetic mice that were either wasting (W, n=4) or not wasting (NW, n=4). Panels e and f are representative sections of pancreata from treated wasting (e) and non-wasting (f) mice. Panel g shows the insulin area in non-diabetic mice that were either untreated (shaded), or treated (open). Panels h and i are representative sections of pancreata from these non-diabetic untreated (h) and treated (i) mice.

FIG. 13. Cachexia in C57BL/6 mice induced by LL2 is also associated with lymphopenia. C57BL/6 mice were either injected with 5×10⁵ LL2 cells in the left thigh or left untreated. Equal numbers of mice from each group were sacrificed at day 15 (n=4), 22 (n=4), 25 (n=2), 27 (n=2) and 28 (n=2) days post LL2 injection and skeletal muscle was isolated and immediately weighed. The data in panel a shows the mean muscle weight±SD for each group at each time point. The number of CD4⁺ T cells in spleen and lymph nodes was determined on days 27 and 28 post-LL2 treatment and compared to age matched untreated control mice. Panel b shows mean±SD of pooled data from days 27 and 28, n=4 per group. The data are representative of two separate experiments. The level of statistical significance is indicated as * for p=0.05-0.01.

FIG. 14. CD4⁺ CD44^{v.low} cells inhibit muscle atrophy in cancer cachexia. C57BL/6 mice were treated on day 0 with LL2 and on the same day (panel a), either, an infusion of CD4⁺ CD44^{v.low} cells (open box, n=5), or, CD4⁺ cells depleted of CD44^{v.low} cells (dots, n=3), or, with no CD4⁺ cells (closed box, n=4). In a separate experiment (panel b), C57BL/6 mice were treated on day 0 with LL2 and then on days 24 and 25 with either CD4⁺ CD44^{v.low} cells (open box, n=4), or, with no CD4⁺ cells (closed box, n=4). Mice in both experiments were sacrificed on day 28 and skeletal muscle weighed. The data for each experiment and are shown as mean±SEM. The level of statistical significance is indicated as * for p=0.05-0.01.

FIG. 15. CD4⁺ CD44^{v.low} cells inhibit skeletal muscle protein and DNA loss in mice with cancer. C57BL/6 mice were treated on day 0 with LL2 and, on the same day, either an infusion of CD4⁺ CD44^{v.low} cells (open box, n=4), or, CD4⁺ cells deplated of CD44^{v.low} cells (dots, n=4), or, with no CD4⁺ cells (closed box, n=4). Mice from each group were sacrificed on days 25 (n=2) and 27 (n=2) days post-LL2 injection, the skeletal muscle was isolated and the total amount of soluble protein (a), and DNA (b) in each muscle was determined. The data are shown as mean±SEM and are pooled from both time points, and is representative of two separate experiments. The level of statistical significance is indicated as * for p=0.05-0.01.

FIG. 16. CD4⁺ CD44^{v.low} cell-mediated protection from cachexia is associated with protection CD4⁺ T cell lymphopenia. On days 27 (n=2) and 28 (n=2) days post-LL2 injection, lymph nodes were isolated from C57BL/6 mice that were treated on day 0 with LL2 and on the same day, either an infusion of CD4⁺ CD44^{v.low} cells (open box), or, CD4⁺ cells depleted of CD44^{v.low} cells (dots), or, with no CD4⁺ cells (closed box). The hatched boxes represent age and sex matched untreated mice. The number of CD4⁺ T cells that expressed CD44 at a very low (CD4⁺ CD44^{v.low}, panel a), intermediate (CD4⁺ CD44^{int}, panel b) and high (CD4⁺ CD44^{high}, panel c) density was determined. The data are shown as mean±SEM and are pooled from both time points. The level of statistical significance is indicated as * for p=0.05-0.01.

FIG. 17. Representative sample of blood collected from a healthy donor. A is a dot plot of CD4 versus CD44 expression of events within a gate that excludes debris. Box 1 shows the region containing beads. Box 2 shows the region containing CD4+ cells. Panel B is a histogram gated on CD4+ cells (Box 2) showing the expression of CD44. Marker, M1 shows CD44^{v.low} cells, M2 shows CD44int cells, and M3 shows CD44high cells.

### DETAILED DESCRIPTION OF PREFERRED EMBODIMENTS

Disclosed herein is the unexpected discovery that the only CD4⁺ cell subset that is significantly lost between the onset of diabetes and the onset of cachexia, is the subset that expressed the lowest density of CD44 (CD44^{v.low}), suggesting that the loss of this cell subset is specific to the development of cachexia. Also disclosed herein is the discovery that CD4⁺ CD44^{v.low} cells delay the onset of wasting. These cells significantly reduced muscle atrophy, inhibited muscle protein loss, and prevented DNA loss, even when given after the onset of cachexia. Protection from wasting and muscle atrophy by CD4⁺ CD44^{v.low} cells was associated with protection from lymphopenia.

Also disclosed herein is the discovery that CD4+ CD44^{v.low} cells can be detected in PBL of healthy blood donors and that the loss of CD4⁺ CD44^{v.low} cells in peripheral blood can be used to predict the onset of cachexia. In addition, CD4+ CD44^{v.low} cells in peripheral blood can be used as a biomarker to indicate cachexia or the onset of cachexia.

Also disclosed herein is the discovery CD4+ CD44^{v.low} cells can be used to treat diabetes by promoting an increase in insulin secretion. Further disclosed herein is the discovery that CD4⁺CD44^{v.low} cells in the PBL of patients with TID can also be used as a biomarker to indicate the onset (increase in CD4⁺CD44^{v.low} cells) and loss (decrease in CD4⁺ CD44^{v.low} cells) of the honeymoon period.

In addition, disclosed herein is the discovery that CD4+ CD44^{int} can differentiate to become CD4+ CD44^{v.low} cells, which provides novel therapeutic approaches to treating and/or diagnosing cachexia, diabetes, and/or diseases associated with cachexia.

The section headings used herein are for organizational purposes only and are not to be construed as limiting the subject matter described.

As described herein, it is intended that where a range of values is provided, it is understood that each intervening value, to the tenth of the unit of the lower limit unless the context clearly dictates otherwise, between the upper and lower limit of that range and any other stated or intervening value in that stated range is contemplated and encompassed within the embodiments. The upper and lower limits of these smaller ranges may independently be included in the smaller ranges is also encompassed within the embodiments, subject to any specifically excluded limit in the stated range. Where the stated range includes one or both of the limits, ranges excluding either or both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein may also be used in the practice or testing of the embodiments, the preferred methods and materials are now described. All publications mentioned herein are expressly incorporated by reference in their entireties.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

In some contexts, the terms “individual,” “host,” “subject,” and “patient” are used interchangeably to refer to an animal that is the object of treatment, observation and/or experiment. “Animal” includes vertebrates and invertebrates, such as fish, shellfish, reptiles, birds, and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

In some contexts, the terms “ameliorating,” “treating,” “treatment,” “therapeutic,” or “therapy” do not necessarily mean total cure or abolition of the disease or condition. Any alleviation of any undesired signs or symptoms of a disease or condition, to any extent, can be considered amelioration, and in some respects a treatment and/or therapy.

The term “therapeutically effective amount/dose” is used to indicate an amount of an active compound, or pharmaceutical agent (including CD4⁺ CD44^{v.low} T cells) that elicits a biological or medicinal response. This response may occur in a tissue, system, animal or human and includes alleviation of the symptoms of the disease being treated. For example, with respect to the treatment of cachexia, a therapeutically effective amount preferably refers to the amount of a therapeutic agent (e.g., CD4⁺ CD44^{v.low} T cells) that reduces or ameliorates symptoms of cachexia or increases the quality life of a patient or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

By “isolated” when referring to T cells (e.g., CD4⁺ CD44^{v.low} T cells), is meant that the indicated cell is present in excess in comparison to other cell types, and preferably that the indicated cell represents the majority of cells present. However, the isolated T cells may include some additional cell types, which do not deleteriously affect the basic characteristics of the composition.

As used herein, the term “purified” refers to samples in which particular populations of CD4⁺ CD44^{v.low} cells are at least 10⁰/s or 20%, preferably 30% or 40% or more preferably 50% free from other components with which they are naturally associated. As used herein, the term “enriched” refers to samples in which the proportion of CD4⁺ CD44^{v.low} cells to other T cells is at least double, preferably 3 times, 5 times, 7 times 10 times, 15 times or 20 times that which occurs in a natural environment.

The terms “vector”, “cloning vector”, “expression vector”, and “helper vector” refer to the vehicle by which a DNA or RNA sequence (e.g., a foreign gene) can be introduced into a host cell, so as to promote expression (e.g., transcription and/or translation) of the introduced sequence. Vectors include plasmids, phages, viruses, pseudoviruses, etc.

The phrase “gene transfer” or “gene delivery” refers to methods or systems for reliably inserting foreign DNA into host cells.

As used herein, the term “transfection” is understood to include any means, such as, but not limited to, adsorption, microinjection, electroporation, lipofection and the like for introducing an exogenous nucleic acid molecule into a host cell. The term “transfected” or “transformed”, when used to describe a cell, means a cell containing an exogenously introduced nucleic acid molecule and/or a cell whose genetic composition has been altered by the introduction of an exogenous nucleic acid molecule.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### T Cells

Embodiments herein relate to isolated T cells. In some embodiments, the T cells are CD4+ T cells. In other embodiments, the T cells are CD4+ CD44^{v.low} T cells.

T cells (e.g., CD4⁺ T cells or CD4⁺ CD44^{v.low} or CD4⁺ CD44^{int.}) can be obtained from a number of sources (e.g., cell samples or biological samples), including but not limited to, blood, peripheral blood leukocytes (PBLs), peripheral blood mononuclear cells, bone marrow, thymus, tissue biopsy, tumor, lymph node tissue, gut associated lymphoid tissue, mucosa associated lymphoid tissue, spleen tissue, or any other lymphoid tissue, and tumors. T cells can be obtained from T cell lines and from autologous or allogeneic sources. T cells may also be obtained from a xenogeneic source, for example, from mouse, rat, non-human primate, and pig

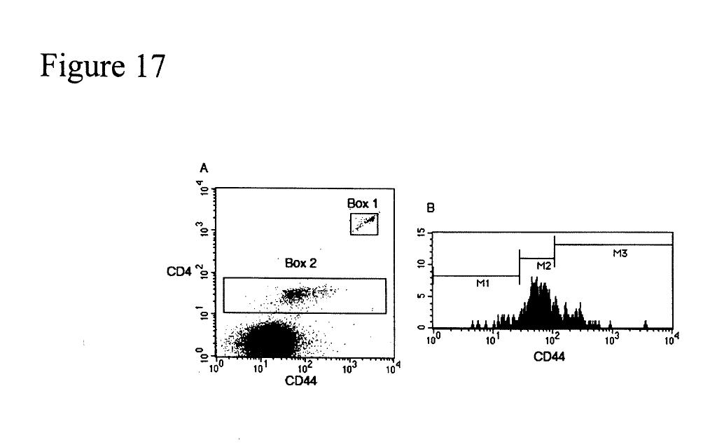

In some embodiments, the cell sample or biological sample is a blood sample and once collected from the patient, peripheral blood leukocytes (PBLs) can then be isolated from the blood sample and stained, for example, with antibodies such as anti-CD4, anti-CD44, or any other antibodies specific to cell markers identified on CD4⁺ CD44′^{v.low} cells or any combination of these antibodies. Then an isolation and/or sorting method, such as, for example, flow cytometry (e.g., fluorescence activated cell sorting (FACS)), bead chromatography, or any other isolation and/or sorting method known in the art can be used to obtain CD4⁺ CD44^{v.low} cells. In some embodiments, CD4+ CD44v.low cells can be identified as the peak with the lowest mean fluorescence intensity as shown and described in FIG. 17. In some embodiments the number of CD4+ CD44v.low cells is less than or equal to about 5% of the total CD4+ T cells present.

In some embodiments the isolated CD4⁺ CD44^{v.low} T cells can be expanded ex vivo or in vitro using any cell growth enhancing environment. CD4+ CD44v.low cell can be expanded in culture with CD3-specific monoclonal antibody with and without the cytokines IL-2 and IL-7 and the CD28-specific monoclonal antibody. Murine CD4+ CD44v.low cells can also be expanded in immunodeficient mice. In some embodiments, human CD4+ CD44v.low cells can be grown in mice that express human HLA antigens. In some embodiments, isolated CD4+ T cells that have an intermediate density of CD44 (CD4+ CD44^{int}), and that do not contain any Foxp3+ cells, are able to differentiate into CD4+ CD44^{v.low} cells. In some embodiments, once the desired population of T cells has been grown up, they can then be transferred into the patient through any pharmaceutically acceptable route. Ex vivo administration, in which cells are isolated from a patient, optionally expanded or altered, optionally purified, and then reintroduced into a patient, is particularly contemplated.

In other embodiments, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis or leukapheresis may be washed to remove the plasma fraction and to place the cells in an appropriate buffer or media for subsequent processing steps. In one embodiment, the cells are washed with phosphate buffered saline (PBS). As those of ordinary skill in the art would readily appreciate, a washing step may be accomplished by methods known to those in the art, such as by using a semi-automated “flow-through” centrifuge (for example, the Cobe 2991 cell processor, Baxter) according to the manufacturer's instructions. After washing, the cells may be resuspended in a variety of biocompatible buffers. Alternatively, the undesirable components of the apheresis sample may be removed and the cells may be directly resuspended in culture media.

In alternative embodiments, T cells are isolated from peripheral blood lymphocytes by lysing the red blood cells, isolating and reserving the monocytes as described previously, or for example, by centrifugation through a PERCOLL™ gradient. A specific subpopulation of T cells, such as CD4+ CD44^{v.low} cells, can be further isolated by positive or negative selection techniques. For example, CD4+ CD44^{v.low} cells can be positively selected using antibody-conjugated magnetic beads (e.g., DYNABEADS™). In one aspect, enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected.

In further embodiments, paramagnetic particles of a size sufficient to be engulfed by phagocytotic monocytes can be used, that are subsequently removed through magnetic separation. In certain embodiments, the paramagnetic particles are commercially available beads, for example, those produced by Dynal AS under the trade name Dynabeads™. Exemplary Dynabeads™ in this regard are M-280, M-450, and M-500. In one aspect, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be expanded. In certain embodiments, the irrelevant beads include beads coated with sheep anti-mouse antibodies, goat anti-mouse antibodies, and human serum albumin.

In some embodiments, the T cells may be genetically modified using any number of methods known in the art. The T cells may be transfected using numerous RNA or DNA expression vectors known to those of ordinary skill in the art. Genetic modification may comprise RNA or DNA transfection using any number of techniques known in the art, for example electroporation (using e.g., the Gene Pulser II, BioRad, Richmond, Calif), various cationic lipids, (LIPOFECTAMINE™, Life Technologies, Carlsbad, Calif), or other techniques such as calcium phosphate transfection as described in Current Protocols in Molecular Biology, John Wiley & Sons, New York. N.Y. For example, 5-50 μg of RNA or DNA in 500 μl of Opti-MEM can be mixed with a cationic lipid at a concentration of 10 to 100 μg, and incubated at room temperature for 20 to 30 minutes. Other suitable lipids include LIPOFECTIN™, LIPOFECTAMINE™. The T cells may also be transduced using viral transduction methodologies as described below The T cells may alternatively be genetically modified using retroviral transduction technologies. In some embodiments, the retroviral vector may be an amphotropic retroviral vector, preferably a vector characterized in that it has a long terminal repeat sequence (LTR), e.g., a retroviral vector derived from the Moloney murine leukemia virus (MoMLV), myeloproliferative sarcoma virus (MPSV), murine embryonic stem cell virus (MESV). murine stem cell virus (MSCV), spleen focus forming virus(SFFV), or adeno-associated virus (AAV). Most retroviral vectors are derived from murine retroviruses. Retroviruses adaptable for use in accordance with the present invention can, however, be derived from any avian or mammalian cell source. These retroviruses are preferably amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

### Treatments

Embodiments disclosed herein relate to methods for treating, ameliorating, preventing, or delaying the onset of cachexia and/or lymphopenia by administering isolated T cells to a patient (e.g., CD4+ CD44^{v.low} T cells). Other embodiments relate to treating, ameliorating, preventing, or delaying the onset diseases, conditions or disorders that are typically associated with cachexia and/or lymphopenia that include, but are not limited to, cancer, AIDS, liver cirrhosis, diabetes mellitus (e.g., Type I diabetes), organ failure (e.g., chronic renal failure), chronic obstructive pulmonary disease, chronic cardiac failure, immune system diseases (e.g., rheumatoid arthritis and systemic lupus erythematosus), tuberculosis, cystic fibrosis, gastrointestinal disorders (e.g., irritable bowel syndrome and inflammatory bowel disease), Parkinson's disease, dementia, major depression, anorexia nervosa, an aged condition and sarcopenia. Cachexia can also result from, for example, aging, autoimmunities, chronic viral, bacterial and fungal infection, and end-stage organ failure. Some embodiments relate to a method of treating the various indications of cachexia or a disease, condition or disorder that is typically associated with cachexia. In particular, some embodiments are related to a method of treating a patient suffering from symptoms of cachexia or a disease, condition or disorder that is typically associated with cachexia. Symptoms of cachexia include, but are not limited to, loss of weight, muscle atrophy, fatigue, weakness, significant loss of appetite, asthenia, and anemia.

Some embodiments relate to newly isolated populations of CD4⁺ T cells that express a low density of CD44, termed CD4⁻ CD44^{v.low} cells. In some embodiments, the newly isolated CD4⁺ CD44^{v.low} cells can be used to treat or prevent the onset of cachexia and/or lymphopenia.

Some embodiments relate to methods for treating or preventing the onset of cachexia and/or lymphopenia by first identifying a patient with cachexia and/or lymphopenia, with a disease, condition or disorder that is typically associated with cachexia and/or lymphopenia, at risk for cachexia and/or lymphopenia, or at risk for a disease, condition or disorder that is typically associated with cachexia and/or lymphopenia, and collecting a cell sample from the patient. In some embodiments, the CD4⁺ CD44^{v.low} can be administered to the same patient from which they were obtained. In other embodiments, the CD4⁺ CD44^{v.low} cells can be administered to a patient other than the patient from which the they were obtained. In still other embodiments, the CD4⁺ CD44^{v.low} cells can be obtained from a mammal that is not a patient. In other embodiments, the administered CD4⁺ CD44^{v.low} cells can comprise a mixture of cells obtained from at least two of the patient to whom the CD4⁺ CD44^{v.low} cells are administered, a patient other than the patient to whom the CD4⁺ CD44^{v.low} cells are administered and a non-patient mammal.

Some other embodiments relate to methods of treating and/or preventing the onset of cachexia and/or lymphopenia or cachexia-related and/or lymphopenia diseases or disorders by activating and expanding certain T cell populations within the body of a patient. For example, agents can be introduced into the body to expand or activate CD4⁺ CD44^{v.low} cells in vivo in a patient with cachexia or at risk of cachexia which results in the amelioration of the effects of the disease or disorder. For example, CD3-specific monoclonal antibody, IL-2, IL-7, and the CD28-specific monoclonal antibody may each be administered alone or in combination. In some embodiments, isolated CD4+ T cells that have an intermediate density of CD44 (CD4+ CD44int), and that do not contain any Foxp3+ cells, can be induced to differentiate into CD4+ CD44^{v.low} cells. In some embodiments, CD4+ CD44v.low cells can be administered to a patient and will expand in vivo to generate more CD4+ CD44v.low cells.

In some other embodiments, the expanded CD4⁺ CD44^{v.low} cell population can be administered alone or in combination with another therapeutic compound. Any therapeutic compound used in the treatment of cachexia or a disease, condition or disorder that is typically associated with cachexia can be used, including but not limited to, hydrazine sulfate, medroxyprogesterone, megestrol acetate, IL-12, melatonin (M. Puccio and L. Nathanson 1997 Seminars in Oncology, 24:277-287), alpha-lipoic acid, amifostine, N-acetyl cysteine (G. Mantovani, et al., 2003 J Mol Med 81:664-673), thalidomide, pentoxyfyline, eicosapentaenoic acid, and ibuprofen (R. Kurzrock 2001 Cancer 92:1684-1688). In one embodiment, no adjuvant is used. In addition, the T cells of embodiments disclosed herein can be administered with agents used to treat the primary disease, including but not limited to, immunosuppressive drugs for the treatment of autoimmunity, anti-cancer drugs, anti-viral drugs, and antibiotics, or any other agent known in the art.

While not being bound to any one particular theory, it is believed that lymphopenia is associated with poor responsiveness to therapy. Thus, reversing lymphopenia in cachexia can be used to promote the responsiveness to therapy (e.g., cancer therapy). Thus, in some embodiments, administering T cells (e.g., CD4⁺ CD44^{v.low}) to a patient or causing the expansion of T cells (e.g., CD4⁺ CD44^{v.low}) in a patient can be used to promote responsiveness to other therapies.

Further embodiments relate to methods of treating, ameliorating or preventing diabetes in a patient. This can be done, for example, by administering T cells (e.g., CD4⁺ CD44^{v.low}) to the patient. Without being bound to any particular theory, it is believed that CD4⁺ CD44^{v.low} T cells can increase insulin-secreting beta cell mass in the pancreas and/or delay the loss of the honeymoon period. This can provide a larger window of time to treat patients who may be more likely to respond to treatments during the honeymoon period.

In addition, CD4⁺ CD44^{v.low} T cells can be used to increase insulin-secreting beta cell mass and to provide a means to grow islets for islet transplantation. In some embodiments, CD4⁺ CD44^{v.low} T cells can be used to promote insulin secretion by islets, either from the patient or other donor, for tansplantation into diabetic patients, irrespective of whether they are cachexic and/or lymphopenic. Therefore, in some embodiments, diabetes can be treated, for example, by growing pancreatic islets in culture using CD4⁺ CD44^{v.low} T cells to increase the function of islet cells and then transplanting them into a patient. In other embodiments, diabetes can be treated by directly administering T cells to the subject.

### Formulations/Pharmaceutical Compositions

Further disclosed herein are pharmaceutical compositions comprising the T cells and a pharmaceutically acceptable carrier. In some embodiments, compositions may be administered either alone, or as a pharmaceutical composition in combination with diluents and/or with other components or cell populations. Briefly, pharmaceutical compositions may comprise a T cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as ethylenediaminetetraacetic acid (EDTA) or glutathione; adjuvants (e.g., aluminum hydroxide); and preservatives.

Pharmaceutical compositions disclosed herein may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “a therapeutically effective amount” is indicated, the precise amount of the compositions to be administered can be determined by a physician with consideration of individual differences in age, weight, extent of disease, and condition of the patient. Typically, in adoptive immunotherapy studies, activated T cells are administered approximately at 2×10⁹ to 2×10¹¹ cells to the patient. (See, e.g., U.S. Pat. No. 5,057,423). In some aspects, particularly in the use of allogeneic or xenogeneic cells, lower numbers of cells, in the range of 10⁶/kilogram (10⁶-10¹² per patient) may be administered. T cell, or other altered post co-culture cell compositions may be administered multiple times at dosages within these ranges. In some embodiments, the dosage of T cells administered to the patient will be from about 10⁶ to about 10¹³ cells, from about 10⁷ to about 10¹² cells, from about 10⁸ to about 10¹¹ cells, or from about 10⁹ to about 10¹⁰ cells. The T cells may be autologous or heterologous to the patient undergoing therapy. This dosage can be repeated as needed on an hourly, daily, weekly, monthly or sporadic basis.

The administration of the subject pharmaceutical compositions may be carried out in any convenient manner, including by aerosol inhalation, injection, ingestion, transfusion, implantation or transplantation. The compositions may be administered to a patient subcutaneously, intradermally, intramuscularly, by intravenous (i.v.) injection, or intraperitoneally. The compositions of T cells may be injected directly into a tissue.

In yet other embodiments, the pharmaceutical composition can be delivered in a controlled release system. In some embodiments, a pump may be used (see Langer, 1990, Science 249:1527-1533; Sefton 1987, CRC Crit. Ref. Biomed. Eng. 14:201; Buchwald et al., 1980; Surgery 88:507; Saudek et al., 1989, N. Engl. J Med. 321:574). In other embodiments, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, N.Y.; Ranger and Peppas, 1983; J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet further embodiments, a controlled release system can be placed in proximity of the therapeutic target, thus requiring only a fraction of the systemic dose (see, e.g., Medical Applications of Controlled Release, 1984, Langer and Wise (eds.), CRC Pres., Boca Raton, Fla., vol. 2, pp. 115-138).

The compositions disclosed herein may also be administered using any number of matrices. Matrices have been utilized for a number of years within the context of tissue engineering (see, e.g., Principles of Tissue Engineering (Lanza, Langer, and Chick (eds.)), 1997. Embodiments disclosed herein utilize such matrices within the novel context of acting as an artificial lymphoid organ to support, maintain, or modulate the immune system, typically through modulation of T cells. Accordingly, embodiments disclosed herein can utilize those matrix compositions and formulations which have demonstrated utility in tissue engineering. The type of matrix that may be used in the compositions, devices and methods of embodiments disclosed herein is virtually limitless and may include both biological and synthetic matrices. In some embodiments, the compositions and devices set forth by U.S. Pat. Nos. 5,980,889; 5,913,998; 5,902,745; 5,843,069; 5,787,900; or 5,626,561 can be utilized, as such these patents are incorporated by reference in their entireties. Matrices comprise features commonly associated with being biocompatible when administered to a mammalian host. Matrices may be formed from both natural and synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release seeded cells or produced cytokine or other active agent. In certain embodiments, the matrix can be flexible and elastic, and may be described as a semisolid scaffold that is permeable to substances such as inorganic salts, aqueous fluids and dissolved gaseous agents including oxygen.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tamports, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

It should be, understood that in addition to the ingredients, particularly mentioned above, the formulations may include other agents conventional in the art having regard to the type of formulation in question.

### Diagnostic and Prognostic Applications

Some embodiments disclosed herein concern diagnostic and prognostic methods for the detection of cachexia, the onset of the honeymoon period in Type I diabetes, and/or the loss of the honeymoon period in Type I diabetes. For example, the detection of levels of CD4+ CD44^{v.low} T cells can provide a means of determining whether or not tissue samples or patients are suffering from cachexia. CD4+ CD44^{v.low} T cell levels may also be used to determine the sensitivity of certain cachexia and/or diabetes treatments or therapies. Such detection methods may be used, for example, for early diagnosis of the disease, to monitor the progress of the disease or the progress of treatment protocols, or to assess the severity of the cachexia. The detection can occur in vitro or in vivo.

The detection of the level of CD4+ CD44^{v.low} T cells may be carried out by any of several means well known to those of skill in the art. Some embodiments disclosed herein relate to methods of detecting S100A6 that is immunological in nature. “Immunological” refers to the use of antibodies (e.g., polyclonal or monoclonal antibodies) to determine the level of CD4+ CD44^{v.low} T cells. For example, antibodies such as anti-CD4, anti-CD44, or any other antibodies specific to cell markers identified on CD4⁺ CD44^{v.low} cells or any combination of these antibodies can be used.

Useful assays include, for example, flow cytometry (e.g., fluorescence activated cell sorting (FACS)), bead chromatography or any other isolation, sorting and/or measuring method can be used to obtain CD4⁺ CD44^{v.low} cells.

As used herein, the term “level” refers to levels of T cells (e.g., CD4+ CD44^{v.low} T cells). Typically the level of the T cells in a biological sample obtained from the patient is different (i.e., increased or decreased) from the “predetermined level” of the same type of cells in a similar sample obtained, for example, at a different time point (examples of biological samples are described herein). For example, the predetermined level may be determined by the control subject data.

In some embodiments, cachexia, the onset of the honeymoon period in Type I diabetes, or the loss of the honeymoon period in Type I diabetes can be diagnosed by assessing whether the level of CD4+ CD44^{v.low} T cells varies from a predetermined level by greater than or equal to 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100%. The pre-determined level and percent variation can be determined using control subject data. This data may vary depending upon the age and sex of the subject.

Numerous well known tissue or fluid collection methods can be utilized to collect the biological sample from the patient in order to determine the level of T cells (e.g., CD4+ CD44^{v.low} T cells). Examples include, but are not limited to, fine needle biopsy, needle biopsy, core needle biopsy and surgical biopsy, lavage, drawing blood, and any known method in the art. Regardless of the procedure employed, once a biopsy/sample is obtained the level of the T cells can be determined and a diagnosis can thus be made. For example, tissue sample may be obtained by collecting blood from a subject. The sample of cells or tissue can then be prepared and exposed to an antibody or a mixture of antibodies according to means which are known to those of skill in the art. Samples can then be prepared for immunohistochemical analysis.

Monitoring Cachexia Therapy and/or Diabetes Therapy

Some embodiments disclosed herein relate to methods for monitoring the progress or efficacy of cachexia therapy and/or diabetes therapy in a subject. The phrase “monitoring the progress of cachexia therapy” or “monitoring the progress of diabetes therapy” refers to determining the relative amount of T cells (e.g., CD4+ CD44^{v.low} T cells) in the body of a patient before, during and/or after cachexia therapy and/or diabetes therapy. In this way, it is possible to evaluate the effectiveness of the therapy. For example, an increase in levels of CD4+ CD44^{v.low} T cells by greater than or equal to 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% will indicate the effectiveness of the therapy. Levels of CD4+ CD44^{v.low} T cells can be measured by methods described herein or by any other method known in the art.

The following examples provide illustrations of some of the embodiments described herein but are not intended to limit invention.

### Example 1

Cachexia in the Non Obese Diabetic Mouse is Associated with CD4⁺ T Cell Lymphopenia

One of the long term consequences of Type I diabetes is weight loss and muscle atrophy, the hallmark phenotype of cachexia. A number of disorders that result in cachexia are associated with immune deficiency. However, whether immune deficiency is a cause or an effect of cachexia is not known. This study examines the non-obese diabetic mouse, the mouse model for spontaneous Type I diabetes, as a potential model to study lymphopenia in cachexia, and to determine whether lymphopenia plays a role in the development of cachexia.

Muscle atrophy seen in patients with Type I diabetes involves active protein degradation by activation of the ubiquitin-proteasome pathway, indicating cachexia. Evidence of cachexia in the non-obese diabetic mouse was determined by measuring skeletal muscle atrophy, activation of the ubiquitin proteasome pathway, and apoptosis, a state also described in some models of cachexia. CD4⁺ T cell subset lymphopenia was measured in wasting and non-wasting diabetic mice.

Data disclosed herein show that the mechanism of wasting in diabetic mice involves muscle atrophy, a significant increase in ubiquitin conjugation, and upregulation of the ubiquitin ligases, MuRF1 and MAFbx, indicating cachexia. Moreover, fragmentation of DNA isolated from atrophied muscle tissue indicates apoptosis. While CD4⁺ T cell lymphopenia is evident in all diabetic mice, CD4⁺ T cells that express a very low density of CD44 were significantly lost in wasting, but not non-wasting, diabetic mice. These data suggest that CD4⁺ T cell subsets are not equally susceptible to cachexia-associated lymphopenia in diabetic mice.

### Introduction

Cachexia (Donnelly and Walsh. Semin Oncol 1995; 22:67-72; Strawford and Hellerstein. Semin Oncol 1988; 25:76-81; Grounds M D. Biogerontology 2002; 3:19-24; Wallace and Schwartz. Int J Cardiol 2002; 85:15-21; Nair et al. J Clin Invest 1995; 95:2926-37) is the dramatic weight loss and muscle atrophy seen in patients with cancer (Donnelly and Walsh. Semin Oncol 1995; 22:67-72) and AIDS (Strawford and Hellerstein. Semin Oncol 1988; 25:76-81) as well as in aging individuals (Grounds M D. Biogerontology 2002; 3:19-24; Wallace and Schwartz. Int J Cardiol 2002; 85:15-21) and in certain autoimmune conditions, including Type I Diabetes (TID) (Nair et al. J Clin Invest 1995; 95:2926-37). TID is an autoimmune disorder caused by the immune-mediated destruction of insulin-secreting pancreatic beta cells, resulting in low insulin production and high blood glucose levels. (Castano and Eisenbarth. Annu Rev Immunol 1990; 8:647-79; Tisch and McDevitt. Cell 1996; 85:291-7). Diabetes can be controlled with daily insulin injections. However, in the long term, diabetes leads to a variety of complications including muscle atrophy and cachexia. (Nair et al. J Clin Invest 1995; 95:2926-37; Charlton and Nair. J Nutr 1998; 128:323S-7S; Vogiatzi et al. J Clin Endocrinol Metab 1997; 82:4083-7). Although significant progress has been made in understanding the pathways that lead to cachexia in the last decade, the majority of these studies use either models of cancer cachexia, or of chemically induced diabetes. The availability of a model of TID cachexia will allow us to determine the relevance of previous studies to TID cachexia. Such a model will also allow further investigation of established pathways, and explore the possibility of novel pathways that are relevant to TID cachexia. In this study, the NOD mouse is used as a model for the study of TID cachexia.

Muscle atrophy in adults with TID (Nair et al. J Clin Invest 1995; 95:2926-37; Charlton and Nair. J Nutr 1998; 128:323S-7S; Vogiatzi et al. J Clin Endocrinol Metab 1997; 82:4083-7), and in rats with chemically-induced diabetes (Liu et al. Biochem Biophys Res Commun 2000; 276:1255-60; Merforth et al. Mol Biol Rep 1999; 26:83-7), as well as muscle atrophy in cancer cachexia (Donnelly and Walsh. Semin Oncol 1995; 22:67-72), involves significant muscle protein loss involving activation of the ubiquitin-proteasome pathway (Price and Mitch. Curr Opin Clin Metab Care 1998; 1:79-83; J Support Oncol 2005; 3:209-17; Am J Clin Nutr 2006; 83:735-43; Curr Opin Clin Nutr Metab Care 2006; 9:220-4). Like patients with TID, the well-established non-obese diabetes (NOD) mouse model for spontaneous TID is also susceptible to weight loss after diabetes onset. (Kikutani and Makino. Adv Immunol 1992; 51:285-322; Makino et al. Jikken Dobutsu 1980; 29:1-213). The mechanism of wasting in the NOD mouse has not yet been reported.

Cachexia is characterized by muscle atrophy and a dramatic loss of muscle protein. (Grounds M D. Biogerontology 2002; 3:19-24; Price and Mitch. Curr Opin Clin Metab Care 1998; 1:79-83; J Support Oncol 2005; 3:209-17; Am J Clin Nutr 2006; 83:735-43; Curr Opin Clin Nutr Metab Care 2006; 9:220-4). A significant loss in muscle DNA has also been described in some models, and this is associated with DNA fragmentation (van Royen et al. Biochem Biophys Res Commun 2000; 270:533-7; Carbo et al. Br J Cancer 2002; 86:1012-6), a classical apoptosis signature. Protein loss in cachexia is the result of a combination of active protein degradation and a decrease in protein synthesis. (Am J Clin Nutr 2006; 83:735-43). Protein degradation involves activation of the ubiquitin-proteasome pathway (N Engl J Med 1996; 335:1897-1905) with an upregulation of the ubiquitin pathway-associated E3 ligases, muscle RING finger 1 (MuRF1) and muscle atrophy F box/atrogin-1 (MAFbx) (Bodine et al. Science 2001; 294:1704-8).

An association between immunodeficiency and cachexia is evident in patients with AIDS (McMichael and Rowland-Jones. Nature 2001; 410:980-7), in ageing individuals (Chakravarti and AbrahamMech Ageing Dev. 1999; 108:183-206; Burns and Goodwin. Drugs Aging 1997; 11:374-97), and in individuals with autoimmunity (Jonsson et al. Scand J Immunol 2002; 56:323-6; Jaramillo et al. Life Sci 1994; 55:1163-77). Whether lymphopenia is a consequence of cachexia, or whether it plays an active role in the development of cachexia is not known. In the event that lymphopenia plays a role in promoting cachexia, then immune intervention might provide a novel therapeutic approach for the treatment of this syndrome. As a first approach in addressing this question, the NOD mouse was used to determine whether the onset of cachexia is association with the preferential loss of a particular CD4⁺ immune cell subset.

A deficiency in CD4⁺ T cells that express a low density of the cell surface marker CD44 (CD44^{low}) is associated with aging (Barrat et al. Res Immunol 1995; 146:23-34; Timm and Thoman. J Immunol 199; 162:711-7), and in the development of spontaneous tumors (Miller et al. J Gerontol 1994; 49:255-62). CD44 is one of the cell surface markers used to distinguish antigen inexperienced (naïve) from antigen experienced (memory) CD4⁺ T cells in the mouse. Naive CD4⁺ T cells express CD44^{low} and a high density of CD62L (CD62L^{high}), while memory cells express CD44 at a high density (CD44^{high}). (Budd et al. J Immunol 1987; 138:3120-9; Swain S L. Immunity 1994; 1:543-52). Naïve cells also express a high density of CD45RB (CD45RB^{high}). (Bottomly et al. Eur J Immunol 1989; 19:617-23; Lee et al. J Immunol 1990; 144:3288-95). By these criteria, the cell subset that is deficient in individuals with spontaneous tumors and during aging is a naïve CD4⁺ T cell. Using these markers to distinguish naïve from memory CD4⁺ T cell subsets, the hypothesis that naïve CD4⁺ T cells are more susceptible to cachexia-associated lymphopenia in diabetic mice than memory CD4⁺ T cells was tested.

The data shows that like muscle atrophy in patients with TID, the mechanism of muscle atrophy in the NOD mouse also involves activation of the ubiquitin-proteasome pathway, suggesting the NOD mouse as a model for the study of TID-induced cachexia. In addition, these findings show that cachexia in TID involved upregulation of both the MAFbx and MuRF1 E3 ligases. While apoptosis was detected in skeletal muscle from wasting diabetic mice, it was only evident as a late event. Having established the NOD mouse as a model for TID cachexia, it is used to show that, both memory and naïve CD4⁺ T cells are lost in diabetic cachexic mice compared to non-diabetic mice. However, the only CD4⁺ cell subset that is significantly lost between the onset of diabetes and the onset of cachexia, is the subset that expressed the lowest density of CD44 (CD44^{v.low}), suggesting that the loss of this cell subset is specific to the development of cachexia. Further investigation will be required to determine whether the loss of this cell subset promotes cachexia and whether inhibiting its loss might provide a novel therapeutic strategy for the treatment of this syndrome.

### Results

### NOD Mice Lose Weight Post-Diabetes Onset

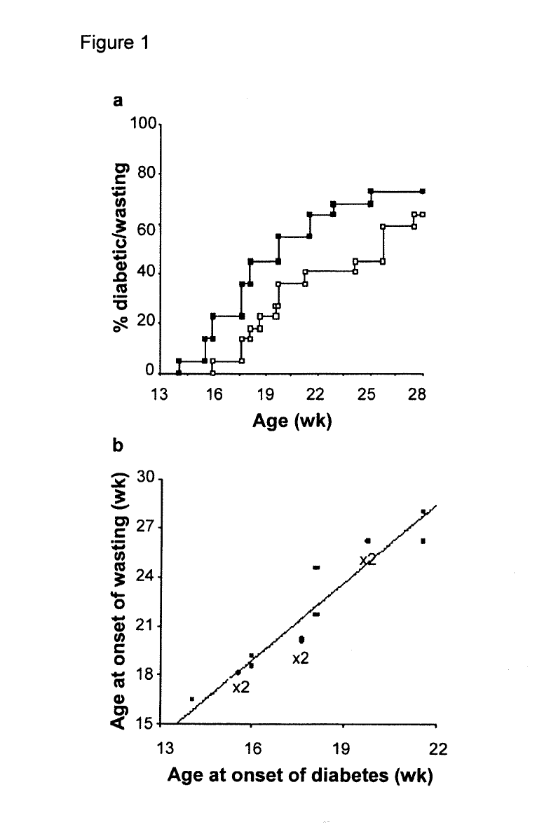

NOD female mice were monitored for diabetes and wasting from the age of ten weeks. Of the twenty-two mice studied, sixteen (74%) became diabetic between fourteen and twenty-five weeks of age. Fourteen of the diabetic mice became wasting between sixteen and twenty-seven weeks of age (FIG. 1a). None of the non-diabetic mice became wasting, and those diabetic mice that were wasting did not lose weight until after the onset of diabetes (FIG. 1b). A linear correlation between the age at onset of diabetes and the age at onset of wasting was found to be highly significant (p<0.0001) with a correlation coefficient of 0.9877 and a 95% confidence interval of 0.9591-0.9963 (FIG. 1b), indicating that the mechanism(s) that results in onset of diabetes and onset of wasting are linked.

Weight Loss in Diabetic NOD Mice is not Associated with a Reduction in Food and Water Intake

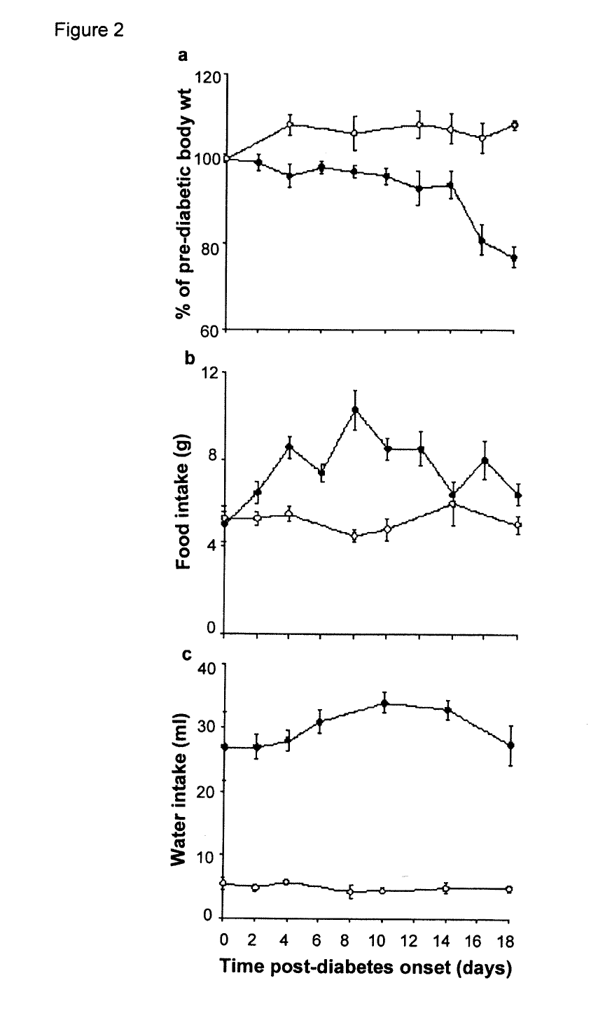

Mice that became diabetic between 14 and 16 weeks of age, and age matched non-diabetic NOD mice were housed separately in individual cages. All mice were weighed every 2-4 days. 400 ml water and 200 g food pellets were measured and given to each diabetic mouse on the day of the second high BGL reading indicating diabetes, and to an equal number of age matched non-diabetic mice. The remaining food and water was measured for each cage 24 hours later. The procedure for measuring food and water intake was repeated every 2-4 days as indicated in FIG. 2. BGL for all mice were taken again at the end of the experiment to confirm the diabetic state of each animal. Only non-diabetic mice that remained non-diabetic for the duration of the experiment are shown as non-diabetic in FIG. 2. As previously shown in FIG. 1, wasting is evident in diabetic mice by 2-3 weeks post-diabetes onset (FIG. 2a). Diabetic mice increase their food intake within the first two weeks after diabetes onset compared to non-diabetic mice. However, food intake is not significantly different in diabetic and non-diabetic mice during the period of weight loss (FIG. 2b). Water intake is also greater in diabetic mice compared to non-diabetic control mice, and this remains higher in diabetic mice than in non-diabetic mice during the period of weight loss (FIG. 2c).

### Skeletal Muscle is Targeted in Wasting NOD Mice

NOD mice were monitored for wasting. Anterior lateral thigh and gastrocnemius muscles were removed from the lower limbs of mice that were either, diabetic and wasting, or neither diabetic nor wasting, and weighed. The weight of both thigh and gastrocnemius muscles were significantly less in mice that were wasting than those from mice that were not wasting, indicating muscle atrophy (Table 1). In addition, when compared to total body weight, skeletal muscle weight was preferentially reduced in wasting mice. This is shown in Table 1 by calculating the ratio of total body weight to skeletal muscle weight.

TABLE 1
Skeletal muscle is preferentially targeted in wasting NOD mice.

| | Wasting^{a} | Non-Wasting^{b} | p value^{c} |
|---|---|---|---|
| Total body wt (g) | 17 +/− 1.9^{d} | 23.9 +/− 1.5 | <0.0001 |
| Right gastro wt (mg)^{e} | 62.9 +/− 15.7 | 117.6 +/− 15.3 | <0.0001 |
| Right thigh wt (mg)^{e} | 81.4 +/− 12.2 | 155.8 +/− 13.3 | <0.0001 |
| Body wt:gastro wt^{f} | 284 +/− 75 | 206 +/− 31 | 0.001 |
| Body wt:thigh wt^{f} | 212 +/− 31 | 160 +/− 18 | <0.0001 |

^{a}Female diabetic NOD mice were monitored for weight loss. Mice were considered wasting when their body weight was reduced by greater than 20% (n = 9).
^{b}Wasting mice were compared to age-matched non-wasting mice (n = 14). Non-wasting mice had lost between 0-5% body weight.
^{c}The Mann-Whitney test was used to determine statistical significance between groups. A p value of less than 0.05 is considered significant.
^{d}The values given are the mean +/− SD for each group.
^{e}The tissue applies to the anterior and lateral thigh muscles isolated from both lower limbs of each mouse.
^{f}The ratio body wt:gastro wt. and body wt:thigh wt. is calculated by dividing the total body weight by the relevant muscle weight.

Wasting in NOD Mice is Associated with Significant Skeletal Muscle Protein and DNA Loss

Cachexia is associated with significant skeletal muscle protein loss, and in some models, DNA loss. Therefore, skeletal muscle was isolated from mice that were wasting (and diabetic) and non-wasting (and not diabetic) and measured both protein and DNA content. Both were significantly reduced in skeletal muscle from wasting mice compared to non-wasting mice (Table 2). These data are consistent with the presence of muscle cachexia.

TABLE 2
Wasting in NOD mice is associated with significant
skeletal muscle protein and DNA loss

| | Wasting^{a} | Non-Wasting^{b} | p value^{c} |
|---|---|---|---|
| Protein^{d} (mg) | 7.0 +/− 1.6^{e} | 19.0 +/− 3.4 | 0.02 |
| DNA^{f} (mg) | 157 +/− 34 | 295 +/− 88 | 0.04 |
| Protein:DNA | 49 +/− 14 | 67 +/− 13 | 0.14 |

^{a}Ten-week old female NOD mice were monitored for the development of wasting. Mice were considered wasting when their body weight was reduced by greater than 20% (n = 8). All mice that were wasting were also diabetic.
^{b}Wasting mice were compared to age-matched non-wasting mice (n = 7). All of the non-wasting mice in this group were non-diabetic.
^{c}The Mann-Whitney test was used to determine statistical significance between groups. A p value of less than 0.05 is considered significant.
^{d}Protein content of gastrocnemius muscle is shown.
^{e}The values given are the mean +/− SD for each group.
g) DNA content of thigh muscle is shown.

Skeletal Muscle Atrophy in NOD Mice is Associated with Apoptosis

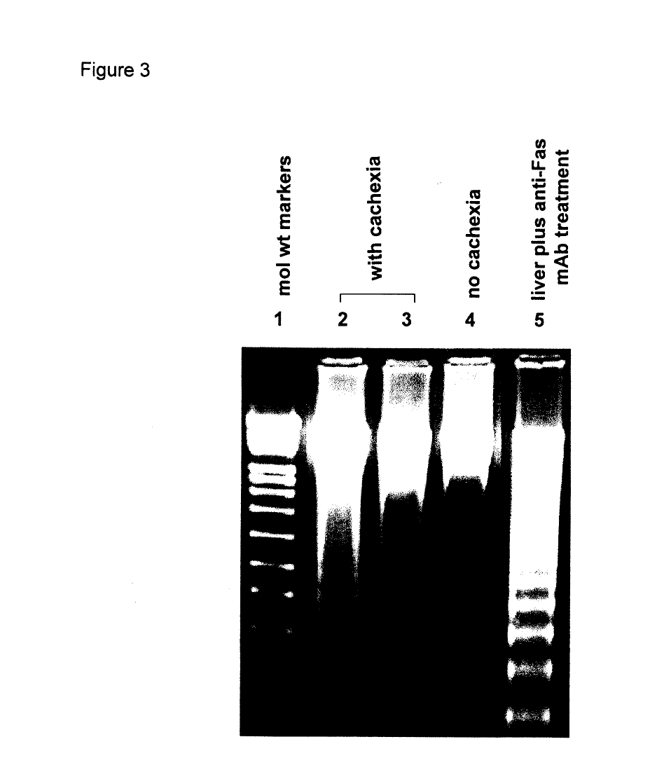

Loss of DNA in muscle cachexia has been associated with apoptosis in the muscle tissue. (van Royen et al. Biochem Biophys Res Commun 2000; 270:533-7; Carbo et al. Br J Cancer 2002; 86:1012-6). Therefore, the possibility that muscle atrophy in NOD mice is associated with apoptosis was directly tested. Skeletal muscle was isolated from five wasting and nine non-wasting mice. DNA was isolated and DNA fragmentation determined. FIG. 3 shows representative samples of DNA from skeletal muscle of wasting and non-wasting mice. Muscle from all wasting mice showed DNA fragmentation while none of the non-wasting mice showed fragmentation (p=0.0005). These data strongly suggest that skeletal muscle wasting in NOD mice involves apoptosis.

Significant Skeletal Muscle Protein Loss is Associated with Wasting and not Diabetes without Wasting

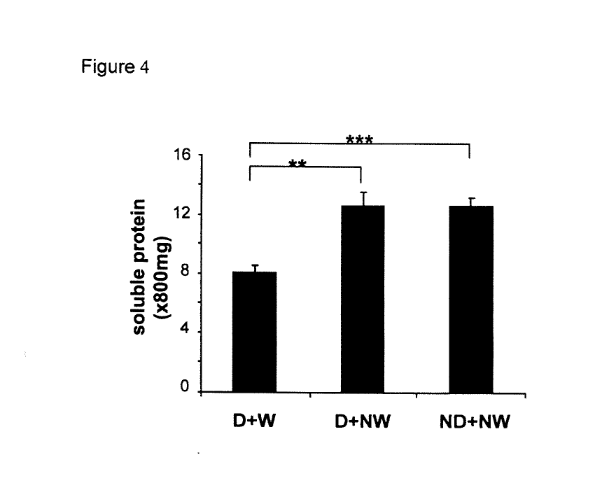

Data shown in Table 1 indicate that muscle atrophy in the wasting diabetic NOD mouse is associated with significant protein loss. In order to confirm that muscle protein was associated with wasting, and not with diabetes in the absence of wasting, skeletal muscle was isolated from mice that were either diabetic and wasting, or diabetic but not wasting, or neither diabetic nor wasting. Significant loss of soluble protein was only detected in muscle isolated from diabetic mice that were wasting and not from mice that were diabetic but not wasting (FIG. 4).

Muscle Atrophy in Diabetic Mice is Associated with a Significant increase in Ubiquitin Conjugation and the E3 ligase MuRF1, but not MAFbx

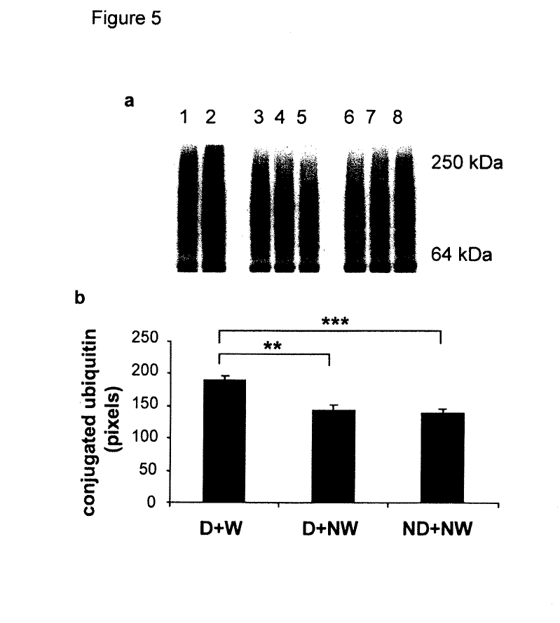

To determine whether protein ubiquitination is associated with muscle atrophy in diabetic NOD mice, the extent of protein conjugated to ubiquitin in skeletal muscle samples from either diabetic and wasting, or diabetic non-wasting, or non-diabetic and non-wasting mice was compared (FIG. 5a). Ubiquitination of high molecular weight proteins (64-250 kDa) is significantly greater in diabetic mice that are wasting compared to diabetic mice that are not wasting (FIG. 5b). Ubiquitination of lower molecular weight proteins (22-64 kDa) is less than that for high molecular weight proteins, and not significantly different between groups (data not shown).

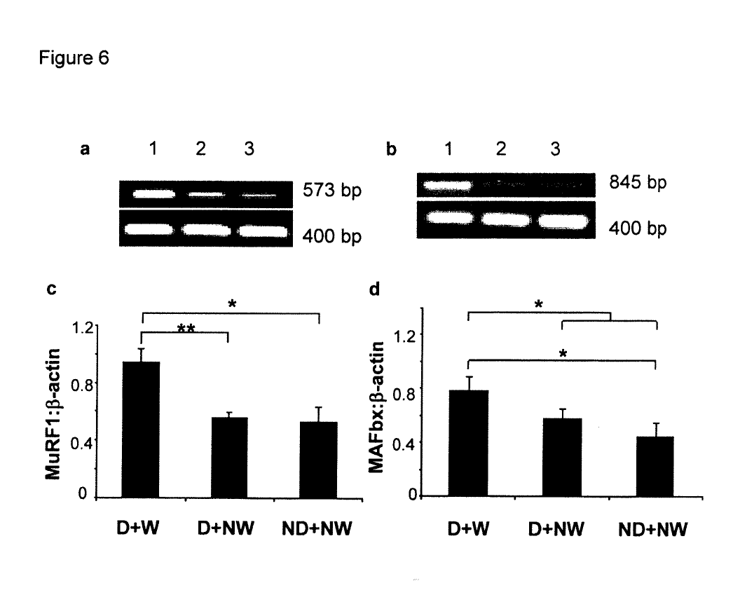

The ubiquitin protein ligases MuRF1 (FIG. 6a) and MAFbx (FIG. 6b) were measured in skeletal muscle from mice in all three groups. Upregulation of MuRF1 was significantly increased at the onset of wasting, but not at the onset of diabetes (FIG. 6c). However, MAFbx upregulation was significantly greater in mice that were diabetic and wasting compared to mice that were non-diabetic and non-wasting, but not compared to mice that were diabetic but not wasting, suggesting that both diabetes and wasting play a role in MAFbx upregulation (FIG. 6d).

### CD4⁺ CD44^{v.low} Cell Deficiency in Cachexic Mice

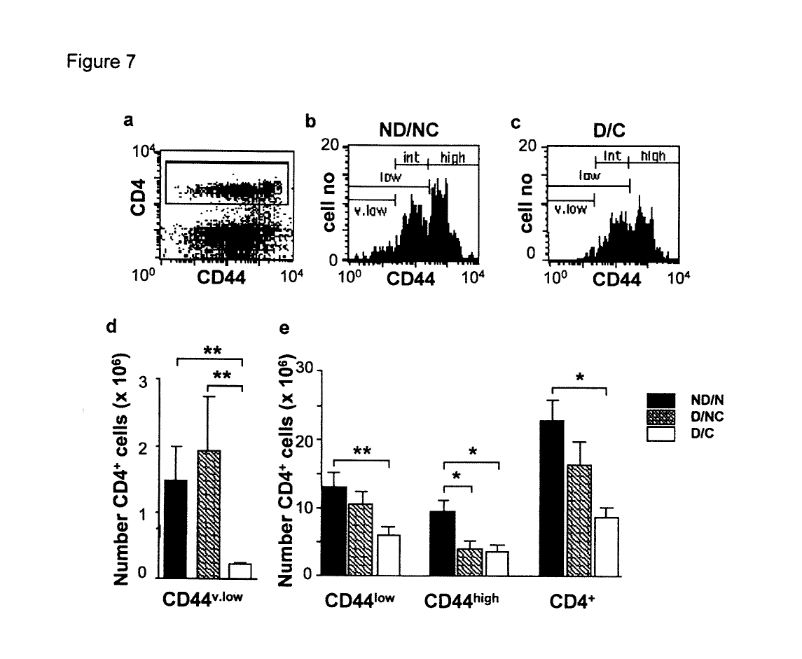

The expression of CD44 on CD4⁺ splenocytes from non-diabetic (ND) and non-cachexic (NC) mice (FIG. 7a) was analyzed in histogram format to define cell subsets by CD44 surface expression (very low, low, intermediate, and high, FIG. 7b). By comparison, the CD44 expression profile from simultaneously diabetic (D) and cachexic (C) mice differed significantly (FIG. 7c), whereas the CD44 expression profile of mice that were diabetic but not cachexic was not different from that shown for non-diabetic mice (data not shown). Strikingly, in diabetic mice that became cachexic (D/C) there was a significant reduction in CD4⁺ CD44^{v.low} cells compared to non-cachexic mice that were either non-diabetic (ND/NC, p=0.002, FIG. 7d) or diabetic (D/NC, p=0.008, FIG. 7d). In addition, the total number of CD4⁺ CD44^{v.low} cells in non-cachexic mice was the same whether the mice were diabetic or not (FIG. 7d). These data suggest the possibility that a deficiency in CD4⁺ CD44^{v.low} cells in the spleen is associated with the development of cachexia but not diabetes.

The data were further analyzed to determine whether cachexia is associated with a deficiency in total naïve CD4⁺ (CD44^{low}) T cells. The total number of CD4⁺ CD44^{low} cells (CD4⁺ CD44^{v.low} plus CD4⁺ CD44^{int}) was not significantly reduced at the onset of diabetes when splenocytes from non-diabetic mice (ND/NC) are compared to splenocytes from diabetic mice (D/NC). In contrast, at the onset of cachexia (D/C) there is a significant reduction in the number of both CD4⁺ CD44^{low} and total CD4⁺ splenocytes compared to splenocytes from non-diabetic mice (FIG. 7e, p=0.008 and p=0.01, respectively). Analysis of the memory CD4⁺ T cell population showed that the total number of CD4⁺ CD44^{high} cells in spleens of cachexic mice was significantly reduced in non-cachexic diabetic mice compared to non-diabetic mice suggesting an association between memory cell deficiency and the onset of diabetes (p=0.03, FIG. 7e). However, a further reduction in the number of memory cells was not detected in the spleens of mice that were also cachexic (D/C) compared to the spleens of diabetic mice that were not cachexic (D/NC).

The phenotype of CD4⁺ CD44^{v.low} Cells

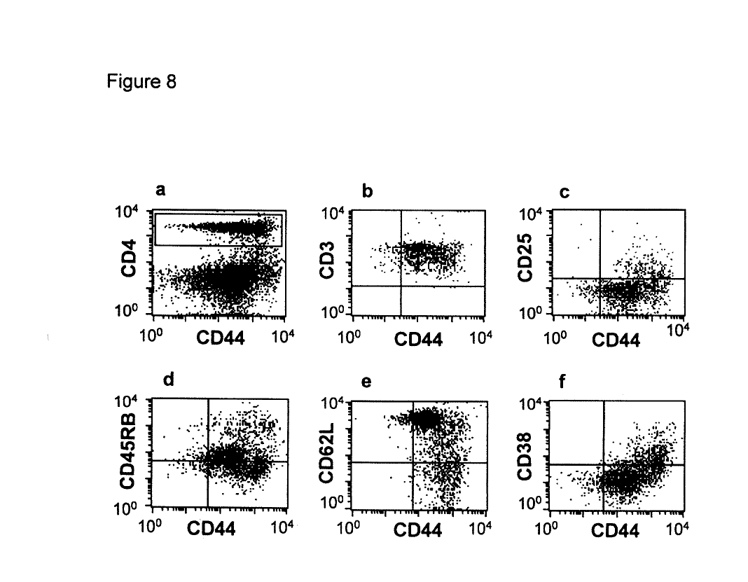

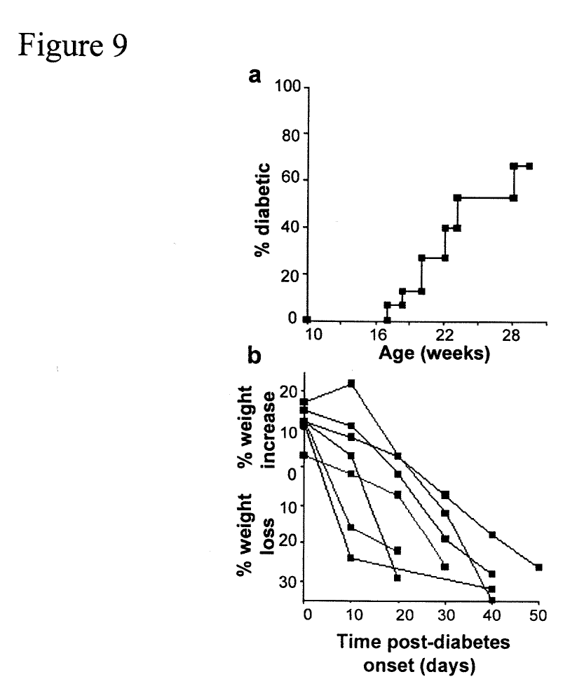

In order to determine whether CD4⁺ CD44^{v.low} cells could be distinguished from naïve CD4⁺ T cells phenotypically, NOD splenocytes were co-labeled with mAb specific CD4 and CD44 (FIG. 8a), and for additional cell surface markers that distinguish CD4⁺ T cell subsets. The data shown in FIG. 9 indicates that, like naïve CD4⁺ T cells, the CD4⁺ CD44^{v.low} cells are CD3⁺ as expected (FIG. 8b), they do not express the regulation/activation marker CD25 (FIG. 8c), they express a mixture of intermediate and high density CD45RB (FIG. 8d), CD62L^{high} (FIG. 8e), and do not express CD38 (FIG. 8f).

### Discussion

One of the long-term complications in patients with TID (Nair et al. J Clin Invest 1995; 95:2926-37; Charlton and Nair. J Nutr 1998; 128:323S-7S; Vogiatzi et al. J Clin Endocrinol Metab 1997; 82:4083-7), and in chemically-induced diabetes in the rat (Liu et al. Biochem Biophys Res Commun 2000; 276:1255-60; Merforth et al. Mol Biol Rep 1999; 26:83-7; Price et al. J Clin Invest 1996; 98:1703-8; Pepato et al. Am J Physiol 1996; 271:E340-7), is muscle atrophy caused by accelerated proteolysis. Wasting in the diabetic NOD mouse, the well-characterized mouse model for spontaneous TID, was also associated with profound skeletal muscle atrophy and a significant loss of skeletal muscle protein and DNA. Activation of the ubiquitin-proteasome pathway is shown by an increase in ubiquitin conjugation of high molecular weight proteins, and upregulation of both MAFbx and MuRF1 in skeletal muscle of wasting diabetic mice. Preferential ubiquitination of high molecular weight proteins has been reported in models of cancer (Combaret et al. Biochem J 2002; 361:185-92), starvation (Wing et al. Biochem J 1995; 307:639-45), and cirrhosis (Lin et al. Am J Physiol Endocrinol Metab 2005; 288: 493-501), and in vitro studies show that high molecular weight proteins are preferentially degraded by the proteasome complex (Wing et al. Biochem J 1995; 307:639-45). Taken together, these data indicate that wasting in the diabetic NOD mouse involves cachexia.

Our data also show that cachexia in diabetic mice, but not diabetic mice in the absence of cachexia, is associated with a significant deficiency in the CD4⁺ CD44^{v.low} T cell subset. CD4⁻ CD44^{v.low} T cell deficiency might be caused either by cell death, or by differentiation to become CD4⁺ CD44^{int} and CD4⁺ CD44^{high} cells. The preferential loss of CD4⁺ CD44^{v.low} cells rather than the equivalent loss of all CD4⁻ T cells suggests either an increased susceptibility of CD4⁺ CD44^{v.low} T cells to cachexia-induced depletion compared to other CD4⁺ cell subsets, or, a role for CD4⁺ CD44^{v.low} T cells in preventing cachexia, resulting in a temporal association between their loss and the onset of cachexia. Further investigation will be required to distinguish between these possibilities.

The co-expression of a high density of CD62L and a mixture of intermediate and high expression of CD45RB, in addition to the low expression of CD44, suggests that the CD4⁺ CD44^{v.low} cells are naïve CD4⁺ T cells. In addition, the lack of expression of the activation markers CD25 (Waldmann T A. Annu Rev Biochem 1989; 58:875-911) and CD38 (Jackson and Bell. J Immunol 1990; 144:2811-5) is consistent with the notion that the CD4⁺ CD44^{v.low} cell subset described here is a resting naïve T cell subset. Both CD25 (Shevach E M. Nat Rev Immunol 2002; 2:389-400; Salomon et al. Immunity 2000; 12:431-40; Diabetes 2007; 56:1395-1402) and CD38 (Martins and Aguas. Immunology 1999; 96:600-5) have also been described as markers that distinguish CD4⁺ cell subsets with regulatory activity in the NOD mouse, suggesting that CD4⁻ CD44^{v.low} cells are not such regulatory cells, and this is also consistent with a naïve cell phenotype. In contrast to the CD4⁺ CD44^{v.low} cells, the naïve CD4⁺ T cell subset as a whole, CD4⁺ CD44^{low} cells, are not significantly reduced at the onset of cachexia in diabetic mice, although they are significantly reduced in cachexic mice when compared to mice that are neither cachexic nor diabetic, suggesting a continuous loss of this cell subset after the onset of diabetes. Memory CD4⁺ T cells, on the other hand, are lost at the onset of diabetes and not at the onset of cachexia. Taken as a whole these data suggest that CD4⁺ T cell lymphopenia in the spleens of cachexic mice is not random, but that the CD4⁺ CD44^{v.low} cell subset is preferentially targeted.

Activation of the ubiquitin-proteasome pathway is common to cachexia seen under a variety of primary disease states, including cancer, chronic infection, diabetes, and starvation (J Support Oncol 2005; 3:209-17; Williams et al. Surgery 1999; 126:744-9), and has become the hallmark that defines wasting as cachexia. The ubiquitin protein E3 ligases, MuRF1 and MAFbx play a critical role in protein degradation by the proteasome pathway. (Bodine et al. Science 2001; 294:1704-8). In the NOD mouse it was found that whereas MuRF1 was significantly upregulated in skeletal muscle of diabetic wasting mice compared to diabetic non-wasting mice, in the case of MAFbx upregulation, significance was only reached when diabetic and wasting mice are compared to non-diabetic and non-wasting mice. These data suggest that MAFbx upregulation begins in diabetic mice before wasting, and then continue as wasting proceeds.

The presence of DNA fragmentation indicates that TID-induced cachexia in the NOD mouse involves apoptosis of skeletal muscle cells. Although evidence of apoptosis has been described in cancer cachexia (van Royen et al. Biochem Biophys Res Commun 2000; 270:533-7; Carbo et al. Br J Cancer 2002; 86:1012-6; Smith and Tisdale. Apoptosis 2003; 8:161-9) it has not been reported in chemically-induced diabetic rats (Lecker et al. FASEB J 2004; 18:39-51). Insulin-like growth factor-1 (IGF-1), a protein that is significantly reduced both in human TID(Capoluongo et al. Eur Cytokine Netw 2006; 17:167-74) and in the diabetic NOD mouse (Landau et al. Int J Exp Diabetes Res 2000; 1:9-18), inhibits caspase 3-mediated apoptosis (Song et al. J Clin Invest 2005; 115:451-8). Therefore, it is tempting to speculate that the mechanism for TID-induced cachexia involves apoptosis by a mechanism that involves a deficiency in IGF-1, and that the NOD model of cachexia provides a model to study mechanisms of apoptosis that are specific to TID cachexia.

A number of factors that play a causal role in the development of diabetes also play a causal role in the onset of cachexia. Thus, insulin can inhibit proteolysis by blocking ubiquitin-mediated proteasomal activity. (Bennett et al. Endocrinology 2000; 141:2508-17) Moreover, treatment of patients with TID with insulin can inhibit protein breakdown. (Charlton and Nair. J Nutr 1998; 128:323S-7S; Abu-Lebdeh and Nair. Baillieres Clin Endocrinol Metab 1996; 10:589-601). However, although treatment with insulin stimulates weight gain in cancer cachexic patients, lean tissue mass was unaffected (Lundholm et al. Clin Cancer Res 2007; 13:2699-706), suggesting that the pathways that lead to cachexia in different primary disease states are not entirely overlapping. In addition, reduced IGF-1 prevents protein breakdown (Curr Opin Clin Nutr Metab Care 2006; 9:220-4) by abrogating proteasome activity in skeletal muscle (Chrysis et al. Growth Horm IGF Res 2002; 12:434-41). Activation of the ubiquitin-proteasomal pathway in the diabetic NOD mouse might also be stimulated by the pro-inflammatory cytokines TNF-α and IFN-γ, which are upregulated during diabetes development (McKenzie et al. Int Immunol 2006; 18:837-46; Skarsvik et al. Scand J Immunol 2004; 60:647-52; Ng et al. Diabetes Res Clin Pract 1999; 43:127-35), and have been shown to stimulate the activation of the ubiquitin proteasomal pathway leading to protein breakdown (N Engl J Med 1996; 335:1897-1905). It is likely that protein breakdown in skeletal muscle of wasting diabetic mice is stimulated by a combination of factors that merge in their action to promote proteaolysis and apoptosis.

To our knowledge, this is the first report that shows that wasting in the NOD mouse model of spontaneous TID is due to cachexia. In addition, it was shown that the mechanism of cachexia in TID involved upregulation of the E3 ligases, MuRF1 and MAFbx. Moreover, like some, but not all, models of cancer cachexia, the mechanism of cachexia in TID also involved apoptosis. These findings suggest that the NOD mouse can be used as a model system to study multiple pathways in the development of TID-induced cachexia. Data generated from experiments designed to distinguish lymphopenia that is associated with diabetes onset, from lymphopenia that is associated with onset of cachexia, suggest that cachexia-associated CD4⁻ T cell lymphopenia is specific to the CD4⁺ CD44^{v.low} cells. Additional investigation will be required to determine whether these cells are a functionally distinct CD4⁺ T cell subset, and whether their loss is an effect of cachexia, or whether the loss of this cell subset plays a role in causing cachexia,. These studies suggest novel therapeutic strategies for the treatment of cachexia in patients with TID.

The example below describes in greater detail some of the materials and methods used in Example 1.

### Example 2

### Mice

Female NOD/LtJ (NOD) adult mice were purchased from the Jackson Laboratories (Bar Harbor, Me.). In our vivarium 70-80% of female NOD mice become diabetic between 14 and 26 weeks of age.

### Assessment of Diabetes

Every week for the duration of the experiment, blood glucose levels (BGL) were tested using a one-step Bayer Glucometer Elite (Bayer, Elkhart, Ind.). Mice that had a BGL of >300 mg/dL were tested again two days later to confirm the high glucose level. Mice were considered diabetic when the BGL were >300 mg/dL over two consecutive readings.

### Assessment of Wasting

Mice were weighed three times a week for the duration of the experiment and were considered wasting when their body weight was 20% less than at the beginning of the experiment. Weight loss in excess of 20% was associated with morbidity and mortality and therefore, wasting mice were sacrificed and tissues taken for analysis within 24 hours of wasting assessment.

### Measurement of Food and Water Intake

Age matched diabetic and non-diabetic NOD mice were housed one mouse per cage. Food weight (g) and water volume (ml) provided to each cage was measured over 24 hr periods every 2 days for the duration of the experiment. The effect of diabetes and wasting on food and water intake was determined.

### Skeletal Muscle Protein Isolation and Quantitation

The left and right gastrocnemius muscles were isolated, weighed, then individually wrapped in autoclaved aluminum foil and stored at −80° C. until analyzed. The packed gastrocnemius muscle was immersed in liquid nitrogen and ground with mortar and pestle. The powdered tissue was transferred into 1 ml of ice-cold homogenization buffer (Tris 0.01M, 2 mM EDTA, 0.15M NaCl, 0.012M Brij 96, 2.22 mM NP-40, 0.025 mM Leupeptin, 0.025 mM Aprotinin, 0.025 mM AEBSF) and homogenized with an electronic pellet pestle. The homogenates were incubated for 30 minutes at 4° C., and centrifuged at 14,000 g for 10 minutes at 4° C. Supernatants were thawed and diluted 1:800 in distilled H₂O on ice. Soluble protein concentration was determined by mixing 160 ml of the diluted sample with 40 ml of Bio-Rad dye reagent (Bio-RAD, Hercules, Calif.) in a 96-well plate using bovine serum albumin (BSA) as the protein standard. Supernatant measurements were performed at least in duplicate. The plates were incubated for 10 minutes at room temperature and read at a 595 nm on a microplate reader (Molecular Devices, Sunnyvale, Calif.).

### Skeletal Muscle DNA Quantitation and DNA Fragmentation

The lateral and anterior thigh muscles were excised from both hind legs of each mouse and weighed. Tissue samples (50 mg) were minced and then lysed in a 6 M guanidinium chloride buffer containing proteinase K (40 μg/ml) at 55° C. for 2-4 hours and then treated briefly with DNase-free RNase following the DNeasy protocol (Qiagen, Valencia, Calif.). Prior to spin column treatment of lysates, small aliquots were diluted in 1M Urea for total DNA measurements using a fluorometric DNA assay (Downs and Wilfinger. Anal Biochem 1983; 131:538-47) with Hoechst dye 33258 (Bio-RAD, Hercules, Calif.). Upon elution of genomic DNA from each spin column, samples were analyzed on a 1.2% agarose gel in 1× TBE containing ethidium bromide and digital images were recorded on an Eagle Eye II UV transilluminator system (Stratagene, La Jolla, Calif.). DNA was similarly isolated from liver tissue of mice treated with anti-Fas mAb (Jo2, Pharmingen, La Jolla, Calif.) for use as a positive control for DNA fragmentation. (Ogasawara et al. Nature 1993; 364:806-9).

### Ubiquitin Conjugation by Western Blot

Protein was isolated from gastrocnemius muscle samples as described above. 30 mg samples of protein supernatant were fractionated by SDS-PAGE (4-20% gradient) and the separated proteins were then transferred onto 0.45 um PVDF (Millipore, Billerica, Mass.). The membranes were blocked with 5% non-fat dry milk and then incubated with anti-ubiquitin polyclonal antiserum (1:100 dilution, Sigma-Aldrich, St. Louis, Mo.) in 5% non-fat dry milk for 2 hours. The blots were washed three times and then incubated with 1:5000 dilution of either anti-rabbit IgG-HRP (Bio-RAD, Hercules, Calif.), or anti-mouse IgG-HRP (Bio-RAD, Hercules, Calif.). After additional washes, the blots were developed with Enhanced ChemiLuminescence (ECL) western blotting detection reagents (Amersham, Piscataway, N.J.), and captured on Hyperfilm ECL (Amersham, Piscataway, N.J.). Ubiquitin conjugated proteins appear as a smear rather than discrete bands. (Minnaugh et al. Electrophoresis 1999; 20:418-28). Therefore, each lane was scanned and the intensity of the smear analyzed using Image J version 1.36b (NIH).

### MuRF1 and MAFbx Measurements by RT-PCR

The gastrocnemius muscle was isolated in the presence of RNAlater RNA Stabilization Reagent (Qiagen, Valencia, Calif.). Total RNA was extracted from 50 mg aliquots of stabilized muscle using TRIzol Reagent (Sigma-Aldrich, St. Louis, Mo.) according to manufacturer's instructions. RNA samples were digested with RNase-free DNase I (Invitrogen, Carlsbad, Calif.) and RT-PCR reactions were performed using Ready-to-go RT-PCR beads (GE Healthcare, Piscataway, N.J.). Briefly, 2 mg RNA and 10 mM of the primers were added to the RT-PCR beads in DEPC water, and the PCR mixtures were subjected to thermal cycling (Bio-RAD, Hercules, Calif.) as follows: 1 cycle of 42 ° C. for 30 minutes for reverse transcription and 95 ° C. for 5 minutes, 32 cycles of 95° C., 1 minute at 55° C. and for 2 minutes, followed by 1 cycle of 75° C. for 5 minutes. For all amplified genes, primers were designed using the Primers3 program, synthesized (Operon, Huntsville, Ala.) and used at a final concentration of 400 mM. The sequences for the primers are as follows: β-actin (400 bp): 5’-TGGAATCCTGTGGCATCCATGAAAC-3′ (forward) and 5′-TAAAACGCAGCTCAGTAACAGTCC-3′ (reverse); MuRF1 (573 bp): 5′-GTCCATGTCTGGAGGTCGTT-3′ (forward) and 5′-GTGGACTTTTCCAGCTGCTC-3′ (reverse); MAFbx (845): 5′-GAACATCATGCAGAGGCTGA-3′ (forward) and 5′-CTTCTTGGCCTGCTGAAAAC-3′ (reverse). PCR products were separated on a 2% agarose gel containing ethidium bromide and gel images were visualized on a UV transilluminator and photographed (Alpha Innotech, San Leandro, Calif.). The intensity of the bands was quantified using Image J version 1.36b (NIH).

### Cell Subset Analysis

Spleen cells from 2-4 month old female NOD mice were prepared for single cell suspensions. Red blood cells were removed with lysing buffer (Sigma Chemical Co., St. Louis, Mo.), and the remaining spleen cells were resuspended in PBS with 1% Fetal Bovine Serum (Intergen Co., New York, N.Y.). Splenocytes were labeled with an allophycocyanin-(APC) conjugated CD4-specific monoclonal antibody (mAb, RM4-5) and PE-conjugated CD44-specific mAb (IM7). APC-conjugated rat IgG2a and PE-conjugated rat IgG2b were used as isotype controls. In some experiments, as indicated in the results section, cells were labeled with APC-conjugated CD4-specific mAb and with either, i) PE-conjugated CD44-specific mAb and FITC-conjugated CD3-specific mAb (hamster IgG, 145-2C11) or, ii) PE-conjugated CD44-specific mAb and FITC-conjugated CD25-specific mAb (rat IgM, OX-39) or, iii) PE-conjugated CD44-specific mAb and FITC-conjugated CD45RB-specific mAb (rat IgG2a, C363.16A) or, iv) PE-conjugated CD44-specific mAb and FITC-conjugated CD38-specific mAb (rat IgG2a, 90) or, v) FITC-conjugated CD44 with PE-conjugated CD62L (rat IgG2a, MEL-14). In each experiment the relevant flurochrome-conjugated isotype controls were used to determine the profile of the positive population. All cell populations were sampled and analyzed using a FACSCalibur with CELLQuest version 3.3 software (Becton Dickinson Immunocytometry Systems, La Jolla, Calif.). All mAbs and isotype controls were purchased from Pharmingen (La Jolla, Calif.).

### Statistical Analysis

The statistical significance for the association of wasting with the loss of skeletal muscle weight, protein and DNA content, and CD4⁺ T cell subsets was assessed using the Mann-Whitney test. (Krauth J. J Neurosci Methods 1983; 9:269-81). The statistical significance of the association of DNA fragmentation, ubiquitin conjugation, E3 ligase upregulation and myosin heavy chain degradation with cachexia was determined using the Unpaired t test. (Ludbrook and Dudley. Aust N Z J Surg 1994; 64:780-7). A linear correlation between the onset of diabetes and the onset of wasting was determined using the Spearman Rank Correlation. (Gaddis and Gaddis. Ann Emerg Med 1990; 19:1462-8). A p value equal to or less than 0.05 is considered significant for all tests. The level of statistical significance is indicated on the Figures as * for p=0.05-0.01, ** for p=0.009-0.001, *** for p=0.0009-0.0001.

### Example 3

### A Novel Role for CD4⁺ T Cells in the Control of Cachexia

Cachexia is the dramatic weight loss and muscle atrophy seen in chronic disease states including autoimmunity, cancer and infection, and is often associated with lymphopenia. It was previously shown that CD4⁺ T cells that expressed the lowest density of CD44 (CD4⁺ CD44^{v.low}) are significantly reduced in diabetic NOD mice that are cachexic compared to diabetic mice that are not cachexic. Using this model, and a model of cancer cachexia, the hypothesis that CD4⁺ CD44^{v.low} cells play an active role in protecting the host from cachexia was tested.

CD4⁺ CD44^{v.low} cells, but not CD4⁺ cells depleted of CD44^{v.low} cells, delay the onset of wasting when infused into either diabetic or pre-diabetic NOD recipients. However, no significant effect on the severity of diabetes was detected. In a model of cancer cachexia, they significantly reduce muscle atrophy, and inhibit muscle protein loss, and DNA loss, even when given after the onset of cachexia. Protection from wasting and muscle atrophy by CD4⁺ CD44^{v.low} cells is associated with protection from lymphopenia. These data suggest, for the first time, a role for an immune cell subset in protection from cachexia, and further suggest that the mechanism of protection is independent of protection from autoimmunity.

### Introduction

Cachexia, characterized by dramatic weight loss and muscle atrophy, is a consequence of a number of chronic disorders including AIDS (Strawford and Hellerstein. 1998. Semin. Oncol. 25:76-81), ageing (Grounds, M. D. 2002. Biogerontology 3: 19-24; Wallace and Schwartz. 2002. Int. J. Cardiol. 85: 15-21; 2007. Clin. Nutr. 26: 389-399) and type 1 diabetes (Nair et al. 1995. J. Clin. Invest. 95: 2926-2937). An association between immunodeficiency and cachexia (McMichael and Rowland-Jones. 2001. Nature 410: 980-981; Chakravarti and Abraham. 1999. Mech. Ageing Dev. 108: 183-206; Burns and Goodwin. 1997. Drugs Aging 11: 374-397; Jonsson et al. 2002. Scand. J. Immunol. 56: 323-326; Jaramillo et al. 1994. Life Sci. 55: 1163-1177) has led us to speculate that a deficiency in the immune system might play a direct role in the development of cachexia, and that correcting or inhibiting the immune deficiency might allow protection.

A deficiency in CD4⁺ T cells that express a low density of the cell surface marker CD44 (CD44¹′) has been associated with aging (Barrat et al. 1995. Res. Immunol. 146: 23-34; Timm and Thoman. 1999. J. Immunol. 162: 711-717; Donnini et al. 2005. Biogerontology. 6:193-204), and with the development of spontaneous tumors (Miller et al. 1994. J. Gerontol. 49: 255-262; Miller et al. 1997. FASEB J. 11: 775-783). In addition, it was shown that a subset of CD4⁺ CD44^{v.low} cells, defined by their expression of the lowest density of CD44 (CD4⁺ CD44^{v.low}), are depleted in diabetic mice at the onset of cachexia, but not in diabetic mice that are not cachexic (Zhao et al. 2008. Immunology. In Press). These data implicate the CD4⁺ CD44^{v.low} T cell subset as a hypothetical candidate for modulating the development of cachexia. CD44 is one of the well-established cell surface markers used to distinguish antigen inexperienced (naïve) from antigen experienced (memory) CD4⁺ T cells in the mouse. Thus, naive CD4⁺ T cells express CD44^{low} and a high density of CD62L (CD62L^{highs}), while memory cells express CD44 at a high density (CD44^{high}) (Budd et al. 1987. J. Immunol. 138: 3120-3129; Swain, S. L. 1994. Immunity 1: 543-552). Naïve cells also express a high density of CD45RB (CD45RB^{high}) (Birkeland et al. 1989. Proc. Natl. Acad. Sci. U.S.A. 86: 6734-6738.; Bottomly et al. 1989. Eur. J Immunol. 19: 617-623; Lee et al. 1990. J. Immunol. 144: 3288-3295). By these criteria, the CD4⁺ CD44^{v.low} T cell subset that is deficient in diabetic mice that are cachexic, is a naïve CD4⁺ T cell.

Autoimmune destruction of pancreatic β cells in type 1 diabetes (TID) results in low insulin production and high blood glucose levels (Castano and Eisenbarth. 1990. Annu. Rev. Immunol. 8: 647-679; Tisch and McDevitt. 1996. Cell 85: 291-297). Without insulin treatment, individuals with TID develop cachexia (Nair et al. 1995. J. Clin. Invest. 95: 2926-2937; Kettelhut et al. 1988. Diabetes Metab. Rev. 4: 751-772). It was shown that wasting in the non-obese diabetic (NOD) mouse, the well-established mouse model for TID (Kikutani and Makino. 1992. Adv. Immunol. 51: 285-322; Makino et al. 1980. Jikken Dobutsu 29: 1-13) is also due to cachexia, with a dramatic loss of skeletal muscle weight, significant muscle protein loss, and activation of the ubiquitin proteasome pathway (Zhao et al. 2008. Immunology. In Press). In addition, muscle atrophy in the NOD mouse is associated with the presence of DNA fragmentation and a significant loss in muscle DNA content, suggesting the possibility that TID cachexia, like some models of cancer cachexia involves apoptosis (Sumi et al. 1999. Osaka City Med. J. 45: 25-35; Carbo et al. 2002. Br. J. Cancer 86: 1012-1016; Busquets et al. 2007. Clin. Nutr. 26: 614-618).

Using the NOD mouse model for TID cachexia, it was found that infusion of highly purified CD4⁺ CD44^{v.low} cells, but not CD4⁺ cells that are depleted of CD44^{v.low} cells, into pre-diabetic NOD mice significantly delays the onset of wasting and muscle atrophy, but no effect on the severity of diabetes was detected. CD4⁺ CD44^{v.low} cells also inhibit muscle atrophy when infused into NOD mice that already have diabetes. That the mechanism of protection induced by CD4⁺ CD44^{v.low} cells is independent of an effect on TID is further suggested by the finding that CD4⁺ CD44^{v.low} cells also inhibit muscle atrophy, including muscle protein and DNA loss, in a C57BL/6 mouse strain model for cancer cachexia. In addition, infusion of CD4⁻ CD44^{v.low} cells, but not CD4⁻ cells depleted of CD4⁻ CD44^{v.low} cells, results in significant inhibition of Lewis lung carcinoma cell (LL2)-induced CD4⁺ T cell lymphopenia in the cancer cachexia model, suggesting that they might modulate cachexia by a mechanism that involves protection from CD4⁺ T cell lymphopenia. To our knowledge, this is the first report that indicates a role for CD4⁺ T cells in protecting the host from muscle atrophy, and suggests a novel role for a CD4⁺ CD44^{v.low} cell subset, and the maintenance of immune homeostasis, in controlling cachexia.

### Results

### Onset of Wasting and Diabetes in NOD Mice

Fifteen NOD female mice were monitored for diabetes and wasting from the age of ten weeks. Ten of the mice were diabetic by twenty-eight weeks of age (FIG. 9a), and seven of these diabetic mice were wasting by seven weeks post-diabetes onset (FIG. 9b). As previously shown (Zhao et al. 2008. Immunology. In Press), none of the five non-diabetic mice became wasting during the time course of the experiment, and the diabetic mice that were wasting did not lose weight until after the onset of diabetes.

CD4⁺ CD44^{v.low} Cells Decrease both the Incidence and the Severity of Wasting, but not Diabetes

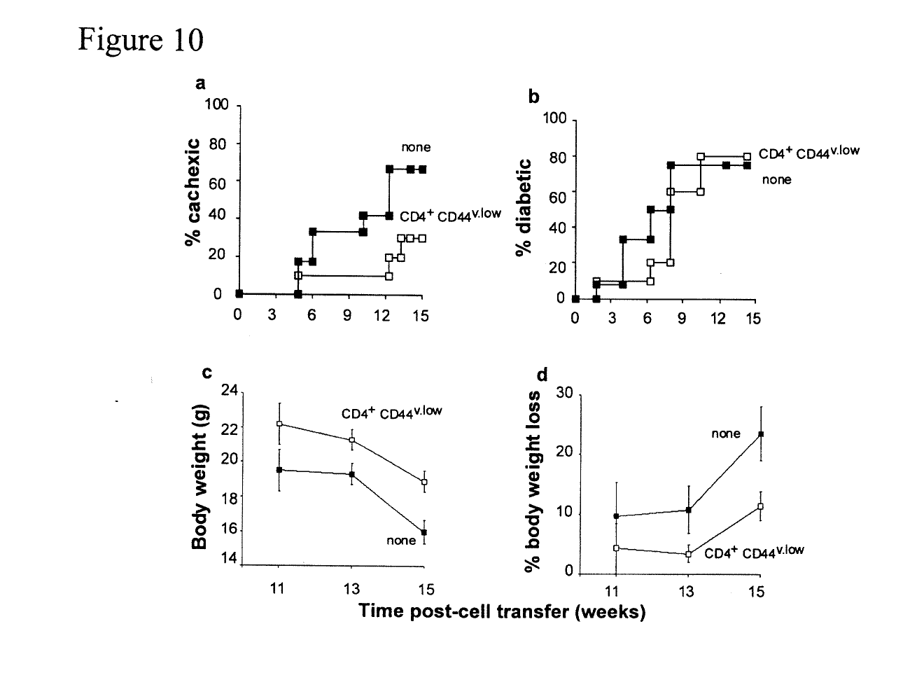

It was previously shown that wasting in the NOD mouse is due to cachexia, and that the onset of cachexia in the diabetic NOD mouse is associated with a significant loss of CD4⁺ CD44^{v.low} cells in the spleen and lymph nodes (Zhao et al. 2008. Immunology. In Press). In order to test the hypothesis that diabetic mice are protected from cachexia by the presence of CD4⁺ CD44^{v.low} cells, the ability of these cells to inhibit the onset of diabetes and wasting in the NOD mouse was first tested. Pre-diabetic female NOD mice were infused with either highly purified CD4⁺ CD44^{v.low} cells or with no cells and then monitored for diabetes and wasting. CD4⁺ CD44^{v.low} cells significantly decrease the rate of onset and the incidence of wasting compared to untreated NOD mice (FIG. 10a, 67% wasting in untreated compared to 30% wasting in CD4⁺ CD44^{v.low} cell treated, p=0.05). In contrast, the infusion of CD4⁺ CD44^{v.low} cells did not inhibit either the onset, or incidence of diabetes (FIG. 10b, 75% diabetic in untreated compared to 80% diabetic in CD4⁺ CD44^{v.low} cell treated). When the total body weight of wasting and non-wasting diabetic mice (from FIG. 10b) was compared (FIG. 10c), and calculated as a percentage of the weight of each mouse at ten weeks of age (FIG. 10d), it was found that CD4⁺ CD44^{v.low} cells significantly inhibited weight loss in diabetic mice (p=0.05 at 13 weeks post-cell infusion, and p=0.03 at 15 weeks post cell infusion for both body weight loss, and % body weight loss), suggesting that CD4⁺ CD440^{v.low} cells decrease the severity as well as the incidence of cachexia (FIGS. 10c and 10d).

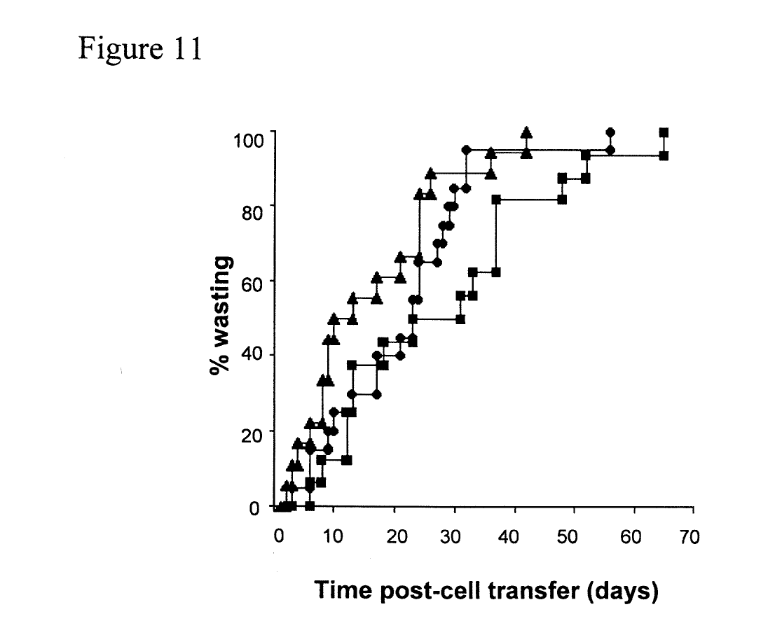

In a separate experiment (FIG. 11) the effect of infusing CD4⁺ CD44^{v.low} cells into mice that were already diabetic was determined, and this effect was compared to the infusion of CD4⁺ T cells that were depleted of the CD4⁺ CD44^{v.low} cell subset. While CD4⁺ CD44^{v.low} cells once again inhibited wasting compared to untreated NOD mice (triangles versus squares in FIG. 11, p=0.02), CD4⁺ cells depleted of CD44^{v.low} cells did not (circles). Taken together the data suggest a relationship between the CD4⁺ CD44^{v.low} cell subset and modulation of cachexia but not TID.

### CD4⁺ CD44^{v.low} Cells Inhibit Muscle Atrophy

In order to confirm that the inhibition of wasting in diabetic NOD mice by CD4⁺ CD44^{v.low} was associated with an inhibition of muscle atrophy, the skeletal muscle was isolated from the CD4⁺ CD44^{v.low} treated and untreated diabetic mice (described in FIG. 10), 15 weeks post cell infusion, and weighed (Table 3). The weight of skeletal muscle from diabetic NOD mice that received CD4⁺ CD44^{v.low} cells was significantly greater than from untreated diabetic NOD mice (179±27 mg in treated compared to 119±9 mg in untreated, p=0.005). Cachexia is associated with preferential loss of skeletal muscle mass (lean tissue mass). That is, in cachexia, the weight of skeletal muscle as a % of total body weight is less than it is in non-cachexic mice. To determine whether CD4⁺ CD44^{v.low} cells protected skeletal muscle preferentially or whether their effect was equivalent in skeletal muscle and the rest of the body weight, skeletal muscle was calculated as a % of total body weight for each mouse and any ability of CD4⁺ CD44^{v.low} cells to preferentially protect skeletal muscle was determined. In untreated non-diabetic mice the skeletal muscle analyzed makes up 1.45±0.06% of the total body weight (Table 3). In contrast, in untreated diabetic mice the same skeletal muscle is reduced to 0.76±0.02% of the total body weight. If the CD4⁺ CD44^{v.low} cells inhibit skeletal muscle weight loss to the same extent as they protect wasting in the rest of the body, % of total body weight that is skeletal muscle should not be significantly different from that seen in untreated mice (0.76±0.02%). However, if CD4⁺ CD44^{v.low} cells preferentially protect skeletal muscle, skeletal muscle in treated mice would be expected to make up a significantly greater % of total body weight than in untreated mice (greater than 0.76±0.02%). Data shows that skeletal muscle weight in treated mice is 0.96±0.12% of total body weight, and is significantly greater than that in untreated mice (0.96±0.12% compared to 0.76±0.02% respectively, p=0.03), suggesting that CD4⁺ CD44^{v.low} cells preferentially inhibit muscle atrophy in diabetic NOD mice (Table 3).

TABLE 3
CD4⁺ CD44^{v.low} cells inhibit skeletal muscle loss

| | Muscle | Body weight | Muscle weight as % |
|---|---|---|---|
| Treatment^{A} | weight (mg)^{B} | (g)^{C} | of total body weight^{D} |
| Diabetic^{E} | | | |
| No treatment | 119 +/− 9^{F} | 15.7 +/− 1.5 | 0.76 +/− 0.02 |
| CD4⁺ CD44^{v.low} | 179 +/− 27 | 18.9 +/− 2.0 | 0.95 +/− 0.12 |
| Non-diabetic | | | |
| No treatment | 374 +/− 20 | 26.3 +/− 1.4 | 1.42 +/− 0.06 |

^{A}Ten-week old female NOD mice were either injected with 2.5 × 10⁵ CD4⁺ CD44^{v.low} cells isolated from eleven week old pre-diabetic NOD donors, or left untreated (no treatment), and monitored for the development of diabetes and wasting.
^{B}Diabetic mice were sacrificed at 25 weeks of age, and skeletal muscle isolated and weighed.
^{C}Mice were weighed at 25 weeks of age.
^{D}Preferential skeletal muscle loss was determined by calculating muscle weight as a percentage of total body weight for each mouse. Untreated non-diabetic mice were used to generate baseline data for muscle weight, and muscle weight as a percentage of total body weight.
^{E}Diabetes is determined as described in Materials and Methods.
^{F}The values given are the mean +/− SD for each group.

### The Effect of CD4⁺ CD44^{v.low} Cells on Insulin-Secreting β Cells

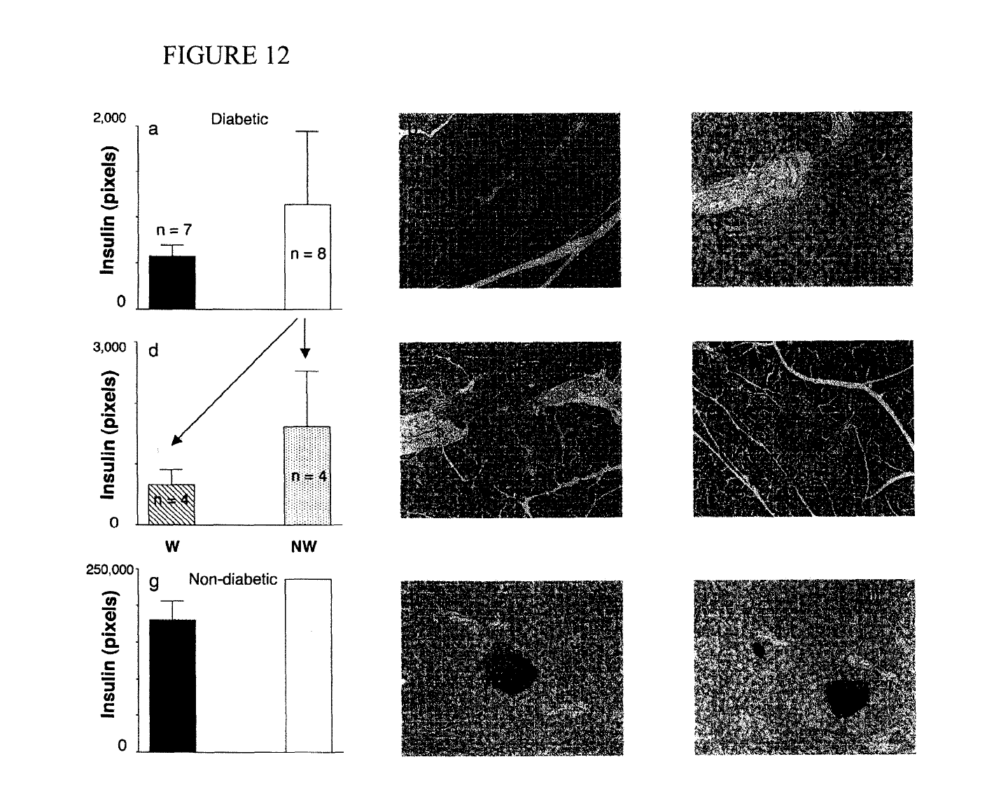

Although data shown in FIG. 10 indicates that CD4⁺ CD44^{v low} cells do not affect the onset of diabetes, it does not exclude the possibility that they might inhibit the loss of insulin-secreting β cells to a level that is sufficient to modulate cachexia, but not diabetes. To address this issue, the effect of CD4⁺CD44^{v.low} cells on insulin-secreting β cell mass in the pancreas was tested. Pre-diabetic NOD mice were treated with and without CD4⁺ CD44^{v.low} cells, and monitored for the development of diabetes and wasting. At 15 weeks post-cell infusion the mice were sacrificed, and the pancreas removed and assayed for the presence of insulin by immunohistochemistry. The relative amount of insulin in each pancreas was determined by measuring the insulin positive area in pixels. The insulin area in the pancreas of diabetic mice that were untreated (7 out of 9 mice were diabetic), was first compared with the insulin area in pancreas of diabetic mice that were treated with CD4⁺ CD44^{v.low} cells (8 out of 9 were diabetic, FIG. 12a). No significant difference was seen in the number of pixels of insulin measured in the pancreata of diabetic untreated (FIG. 12b) and diabetic treated (FIG. 12c) mice. Whereas all of the diabetic mice in the untreated group were also wasting by this time point, only 4 out of 8 diabetic mice in the treated group were wasting. However, although there was a trend that suggested an increase in insulin in the non-wasting group (FIG. 12d) compared to the wasting group (FIG. 12e), no significant difference was seen between these two groups. In addition, there is no difference (FIG. 12g) in the amount of insulin in the non-diabetic untreated (FIG. 12h) and treated (FIG. 12i) mice. The amount of insulin measured in pancreas from treated mice that were diabetic but not wasting was, at best, 1% of that measured in pancreas of non-diabetic treated and untreated mice (FIG. 12g compared to FIGS. 12d respectively).

Cancer Cachexia in C57BL/6 Mice Induced by LL2 is also Associated with Lymphopenia

Although the data thus far suggest that the CD4⁺ CD44^{v.low} cells inhibit cachexia by a mechanism that is independent of any effect on diabetes, this possibility was tested further using a model of cachexia that does not require diabetes as its primary disease. The LL2 model was chosen from a number of models of cancer cachexia because, unlike other models of cancer cachexia, the mechanism of cachexia induced by LL2, like that in the NOD mouse, involves skeletal muscle apoptosis in addition to activation of the ubiquitin proteasome pathway.

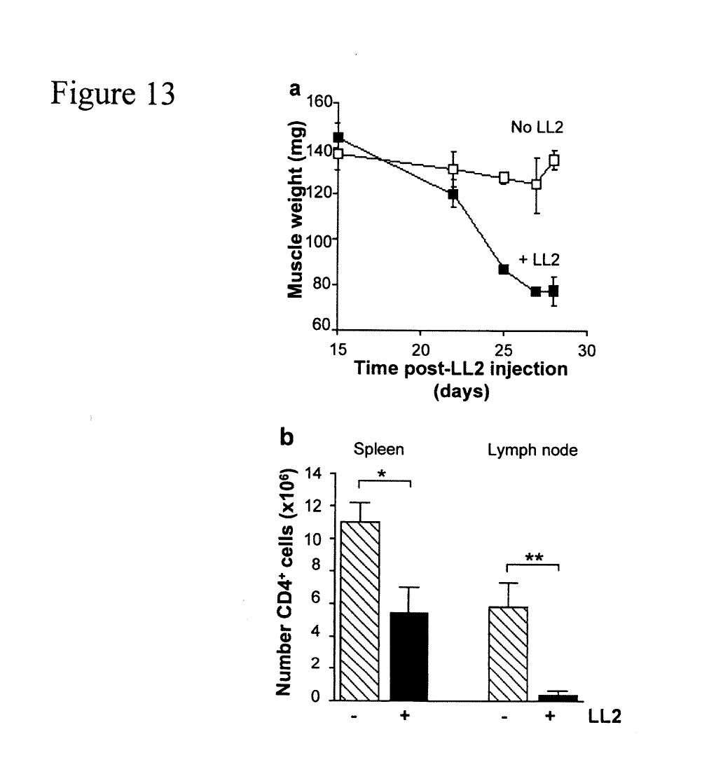

In order to determine whether cachexia in the LL2 model of cancer cachexia is also associated with lymphopenia, C57BL/6 mice were injected with LL2 cells and sacrificed at various times between 15 and 28 days afterward. Skeletal muscle was isolated and weighed. Consistent with published data, significant weight loss was seen in the skeletal muscle of LL2-treated mice by 25 days post-injection compared to age matched control mice (FIG. 13a). CD4⁺ T cell number in both spleens and lymph nodes was significantly reduced in cachexic mice compared to untreated control mice, indicating that the LL2 model for cancer cachexia is associated with CD4⁺ T cell lymphopenia (p=0.02 for spleen, and p=0.04 for lymph node (FIG. 13b).

### CD4⁺ CD44^{v.low} Cells Inhibit Muscle Atrophy in Cancer Cachexia

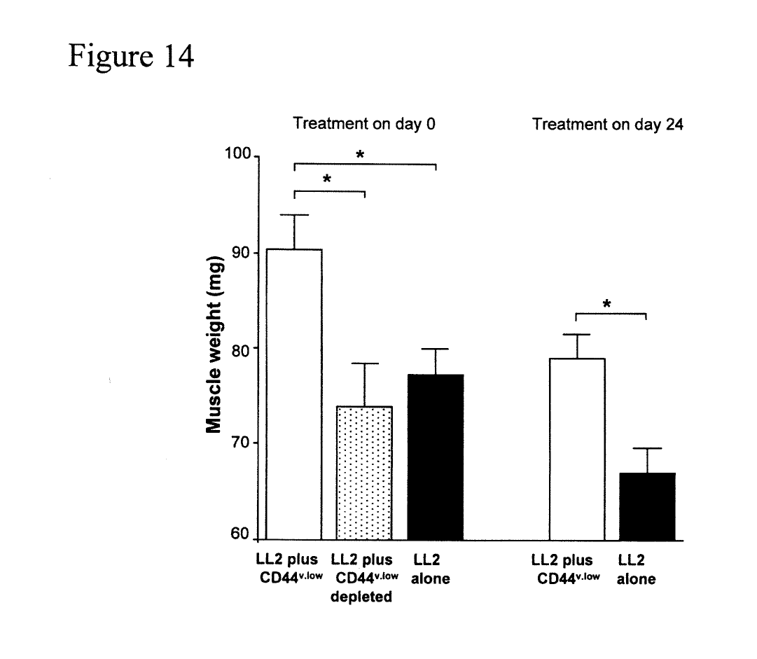

Having established the time of onset of cachexia in the LL2 model, it was determined whether infusion of CD4⁺ CD44^{v.low} cells was also able to modulate cancer cachexia, as it does for diabetes-induced cachexia. C57BL/6 mice were injected with LL2 and on the same day infused with either CD4⁺ CD44^{v.low} cells, or CD4⁺ cells depleted of CD44^{v.low} cells, or with no cells (FIG. 14). Skeletal muscle was removed and weighed on day 28 post-LL2 injection. Skeletal muscle weight was significantly greater in CD4⁺ CD44^{v.low} cell treated mice, compared to mice treated with either CD4⁺ cells that were depleted of CD44^{v.low} cells, or with no cells.

Similar data were obtained when CD4⁺ CD44^{v.low} cells were infused 24 and 25 days days post-LL2 injection, at a time when cachexia was clearly evident. Again the severity of muscle atrophy was significantly reduced in LL2-treated mice that received the CD4⁺ CD44^{v.low} cell infusion compared to mice that did not (FIG. 14).

CD4⁺ CD44^{v.low} Cells Inhibit Skeletal Muscle Protein and DNA Loss in Mice with Cancer

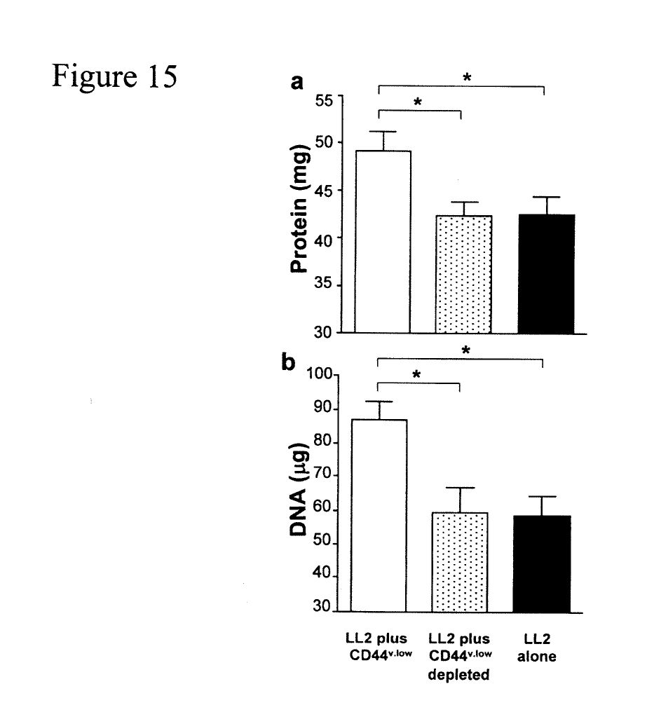

Muscle atrophy in cachexia is associated with a dramatic loss of muscle protein. In addition, the cancer cachexia model used here is also associated with a significant loss in muscle DNA (Carbo et al. 2002. Br. J. Cancer 86: 1012-1016; 2007. Clin. Nutr. 26: 614-618; Gu and Sarvetnick. 1993. Development. 118: 83-46). In order to determine whether protection from muscle atrophy by infused CD4⁺ CD44^{v.low} cells resulted in protection from protein and DNA loss, C57BL/6 mice were injected with LL2 and, on the same day infused with either CD4⁺ CD44^{v.low} cells, CD4⁺ cells depleted of CD44^{v.low} cells, or with no cells. Skeletal muscle taken from mice treated with CD4⁺ CD44^{v.low} cells 28 days after LL2 injection contained significantly more soluble protein (FIG. 15a) and DNA (FIG. 15b) than skeletal muscle taken from mice treated with either CD4⁺ cells depleted of CD44′^{v.low} cells, or with no cells.

CD4⁺ CD44^{v.low} Cell-Mediated Protection from Cachexia is Associated with Protection CD4⁺ T Cell Lymphopenia

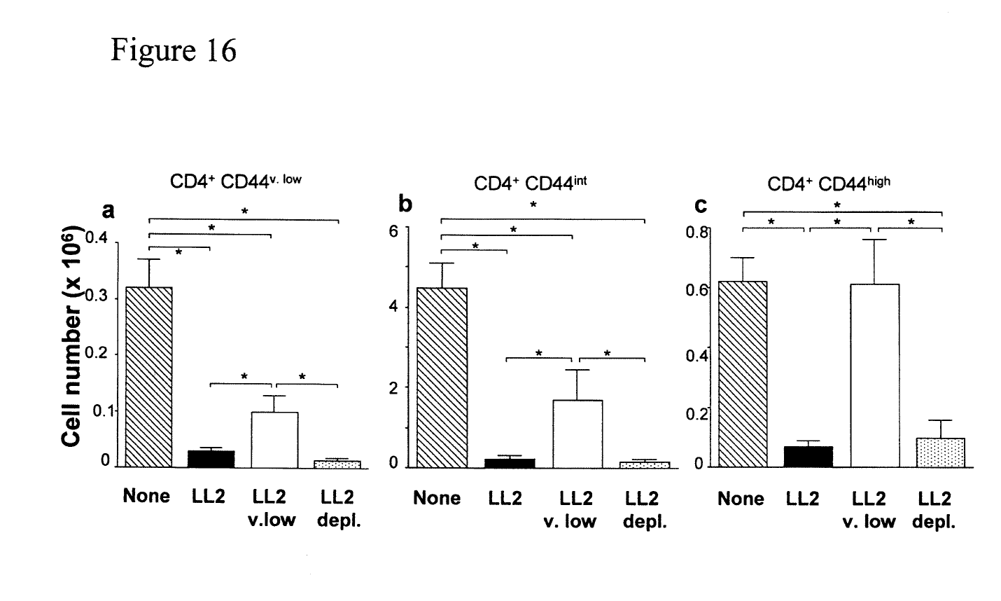

C57BL/6 mice injected with LL2 and infused with either CD4⁺ CD44^{v.low} cells, CD4⁺ cells depleted of CD44^{v.low} cells, or with no cells, were sacrificed 28 days later, and lymph nodes were removed. LL2-treated mice that were infused with CD4⁺ CD44^{v.low} cells, but not CD4⁺ cells depleted of CD44^{v.low} cells, had significantly greater numbers of CD4⁺ CD44^{v.low} (FIG. 16a), CD4⁺ CD44^{int} (FIG. 16b), and CD4⁺ CD44^{high} (FIG. 16c) cells compared to mice that did not receive a CD4⁺ T cell infusion. The effect of CD4⁺ CD44^{v.low} cell infusion on CD4⁺ T cell lymphopenia is similar in the spleen to that seen for lymph node (data not shown). However, data is shown for lymph node rather than spleen because lymphopenia in the lymph node is more dramatic than that seen in the spleen (FIG. 13b). The data show that treatment with CD4⁻ CD44^{v.low} cells can inhibit cancer-induced CD4⁺ T cell lymphopenia, and are consistent with the hypothesis that the mechanism that CD4⁺ CD44^{v.low} cells use to modulate cachexia involves inhibition of CD4⁺ T cell lymphopenia.

### Discussion

Cachexia (Strawford and Hellerstein. 1998. Semin. Oncol. 25:76-81; Grounds, M. D. 2002. Biogerontology 3: 19-24; Wallace and Schwartz. 2002. Int. J. Cardiol. 85: 15-21; 2007. Clin. Nutr. 26: 389-399; Nair et al. 1995. J. Clin. Invest. 95: 2926-2937) is the term used to describe the overall severe weight loss, muscle wasting and anorexia seen in patients with a variety of primary disorders including cancer (Am. J. Clin. Nutr. 83: 1345-1350), AIDS (Strawford and Hellerstein. 1998. Semin. Oncol. 25:76-81), certain autoimmune conditions, including TID (Nair et al. 1995. J. Clin. Invest. 95: 2926-2937), chronic infection (Tracey and Cerami. 1989. Ann. N. Y. Acad. Sci. 569: 211-218) and sepsis (Hasselgren and Fischer. 2001. Ann. Surg. 233: 9-17), and is often present in aging individuals with failure to thrive syndrome (Grounds, M. D. 2002. Biogerontology 3: 19-24; Wallace and Schwartz. 2002. Int. J. Cardiol. 85: 15-21; 2007. Clin. Nutr. 26: 389-399; Chakravarti and Abraham. 1999. Mech. Ageing Dev. 108: 183-206; Burns and Goodwin. 1997. Drugs Aging 11: 374-397). Muscle wasting specifically refers to the loss of muscle mass, preferentially in skeletal muscle. It has important clinical consequences including impaired rehabilitation and shortness of breath (Hasselgren and Fischer. 2001. Ann. Surg. 233: 9-17). Although a number of treatments have been used with some success (2007. J. Support Oncol. 5: 119-125; J. Clin. Oncol. 22: 2469-2476; Nutrition. 24: 305-313) further improvements in therapeutic approaches are needed. In this study the role of CD4⁺ T cells in the control of muscle wasting in animal models of TID and cancer cachexia was focused on.

Cachexia is often associated with lymphopenia, and cachexia in the two primary disease models used in this study is no exception. The presence of lymphopenia in patients with cachexia is associated with decreased responsiveness to therapy and poor prognosis. Whether lymphopenia is a cause or an effect of cachexia is not yet known. Under healthy conditions a balance between naïve and memory T cell numbers (Min et al. 2004. Proc. Natl. Acad. Sci. U.S.A. 101: 3874-3879; 2003. J. Immunol. 1; 171:61-68; 2007. J. Exp. Med. 204: 1665-1675), and the size of the T cell pool (Freitas and Rocha. 1993. Immunol. Today. 14: 25-29; Bell and Sparshott. 1997. Semin. Immunol. 9: 347-353; Mackall et al. Semin. Immunol. 9: 339-346) is maintained at a constant level by homeostatic mechanisms. It was previously shown that lymphopenia in TID is associated with a preferential loss of CD4⁺ CD44^{v.low} cells, but not CD4⁺ CD44^{low} and CD4⁺ CD44^{high} cells, at the onset of cachexia (Zhao et al. 2008. Immunology. In Press). Based on these data it was hypothesized that CD4⁺ CD44^{v.low} cells promote protection from cachexia and that cachexia ensues, at least in part, as a result of their loss. This hypothesis predicts that infusion of CD4⁺ CD44^{v.low} cells into cachexic or pre-cachexic mice might protect from cachexia. Our data show that the transfer of highly purified CD4⁺ CD44^{v.low} cells into pre-diabetic NOD mice significantly inhibit total body weight loss, and skeletal muscle atrophy. In contrast, these cells have no effect on the rate of onset and incidence of TID. Moreover, modulation of cachexia was also seen in mice that were infused with CD4⁺ CD44^{v.low} cells, but not CD4⁺ cells that are depleted of CD44^{v.low} cells, after the onset of diabetes. These data suggest that CD4⁺ CD44^{v.low} cells protect from cachexia but not autoimmune diabetes. However, treatment of TID patients with insulin can inhibit muscle protein breakdown (Charlton and Nair. 1998. J. Nutr. 128: 323S -327S ; Abu-Lebdeh and Nair. 1996. Baillieres Clin. Endocrinol. Metab. 10: 589-601) by inhibiting proteolysis and ubiquitin-mediated proteasomal activity (Bennett et al. 2000. Endocrinology. 141: 2508-2517). Therefore, it was possible that CD4⁺ CD44^{v.low} cells inhibited the autoimmune destruction of insulin secreting β cells to an extent that was insufficient to prevent TID, but sufficient to protect from cachexia. Analysis of the pancreata from diabetic treated, and untreated, mice failed to show a significant effect of CD4⁺ CD44^{v.low} cells on insulin secreting β cells mass in diabetic NOD mice. These data strongly suggest that these CD4⁺ CD44^{v.low} cells modulate cachexia by a mechanism that is independent of an effect on autoimmune diabetes. Moreover, CD4⁺ CD44^{v.low} cells also inhibit wasting and muscle atrophy in a model of cancer cachexia in a non-autoimmune-susceptible mouse strain, further supporting the conclusion that these cells protect from cachexia by a mechanism that is independent of an effect on autoimmunity and insulin secretion. Taken as a whole, the data also suggest that the pathways that lead to cachexia in multiple primary disease states are, at least in part, overlapping. This is consistent with findings that show a common program of changes in gene expression in atrophied skeletal muscle isolated from rats with cancer cachexia and rats with chemically-induced diabetes (Lecker et al. 2004. FASEB J. 18: 39-51). It is important to note that these data do not confirm that the loss of this cell subset is the cause of cachexia in TID and cancer. However, the data do show that CD4⁺ CD44^{v.low} cells are able to protect from cachexia when infused into cachexia or pre-cachexic mice.

Lymphopenia can lead to organ-specific autoimmunity (Gleeson et al. 1996. Immunol. Rev. 149: 97-125; Schaller, J. G. 1975. Birth Defects Orig. Artic. Ser. 11: 173-184), and a resolution of the lymphopenia can result in a resolution of autoimmune pathology (Barthlott et al. 2003. J. Exp. Med. 197: 451-460). However, since skeletal muscle has not been described as a target for autoimmunity, it is unlikely that modulation of cachexia by CD4⁺ CD44^{v.low} cells involves mechanisms that are relevant to protection from autoimmunity that is specific for skeletal muscle. Additional support for the conclusion that the mechanism of protection exerted by CD4⁺ CD44^{v.low} cells on cachexia does not involve protection from autoimmunity comes from the analysis of the phenotype of the CD4⁺ CD44^{v.low} cell population (Zhao et al. 2008. Immunology. In Press). Thus, CD4⁺ CD44^{v.low} cells do not express CD25 (Shevach, E. M. 2002. Nat Rev. Immunol. 2: 389-400; Salomon et al. 2000. Immunity. 12: 431-40; 2007. Diabetes. 56: 1395-1402), CD38 (Martins and Aguas. 1999. Immunology. 96: 600-605), and CD45RB^{low} (Powrie et al. 1993. Int. Immunol. 5: 1461-1471; Fontenot et al. 2003. Nat. Immunol. 4: 330-336), cell surface markers that define regulatory cell subsets known to inhibit autoimmunity. In addition, the regulatory cell marker Foxp3 (Hori et al. 2003. Science 299: 1057-1061; Apostolou and von Boehmer. 2004. J. Exp. Med. 199: 1401-1408) is also not expressed by CD4⁺ CD44^{v.low} cells (J. D. Davies, unpublished observation). Naïve CD4⁺ T cells that do not express regulatory cell markers have been shown to inhibit wasting caused by autoimmune colitis, but not wasting caused by cachexia (Barthlott et al. 2003. J. Exp. Med. 197: 451-460).

CD4⁺ T cells expressing the lowest two densities of CD44 (denoted as CD44^{v.low} and CD44^{int} in this study), are generally considered naïve CD4⁺ T cells (CD44^{low}), while cells that express a high density of CD44 (CD44^{high}) are generally considered memory CD4⁺ T cells (Budd et al. 1987. J. Immunol. 138: 3120-3129; Swain, S. L. 1994. Immunity 1: 543-552). Therefore, based on the density of expression of CD44, CD4⁺ CD44^{v.low} cells are naïve CD4⁺ T cells. CD4⁺ CD44^{v.low} cells also express a high density of CD62L and intermediate/high density of CD45RB (Zhao et al. 2008. Immunology. In Press) further suggesting their naive status (Birkeland et al. 1989. Proc. Natl. Acad. Sci. U.S.A. 86: 6734-6738; Bottomly et al. 1989. Eur. J. Immunol. 19: 617-623; Lee et al. 1990. J. Immunol. 144: 3288-3295). In addition, while activated CD4⁺T cells express CD25 (Waldmann, T. A. 1989. Annu. Rev. Biochem. 58: 875-911) and CD38 (Jackson and Bell. 1990. J. Immunol. 144: 2811-2815), the CD4⁺ CD44^{v.low} cell subset expresses neither CD25 nor CD38 (Zhao et al. 2008. Immunology. In Press), suggesting this cell subset is a resting naïve CD4⁺ T cell. However, our data does not exclude the possibility that this cell subset is not functional in its naïve, unactivated state and that the CD4⁺ CD44^{v.low} T cell subset might only delay cachexia after activation and/or differentiation into a memory cell subset.

In addition to modulating cachexia, CD4⁺ CD44^{v.low} cell infusion significantly reduced the extent of CD4⁺ cell lymphopenia in the CD44^{v.low}, CD44^{int} and the CD44^{high} subsets, protection of CD4⁺ CD44^{high} cell numbers being the most significant. Inhibition of lymphopenia by CD4⁺ CD44^{v.low} cells might result from differentiation and proliferation to CD4⁺ CD44^{int} and CD4⁺ CD44^{high} cells, as well proliferation to maintain the CD4⁺ CD44^{v.low} cell population itself. Alternatively, CD4⁺ CD44^{v.low} cells might inhibit the depletion of CD4⁺ T cell subsets. These data strengthen the association between lymphopenia and cachexia by showing, for the first time, that protection from lymphopenia is associated with protection from cachexia. It is tempting to speculate that the mechanism used by CD4⁺ CD44^{v.low} cells to protect from cachexia involves protection from CD4⁺ T cell lymphopenia, and in particular, protection of the memory cell pool. Alternatively protection from cachexia might not involve protection from lymphopenia. Nevertheless, the finding that CD4⁺ CD44^{v.low} cells protect from lymphopenia might have important clinical implications in improving responsiveness to therapy, irrespective of whether protection from lymphopenia plays a role in protection from cachexia.

The highly consistent balance demonstrated between T cell subsets in healthy individuals might suggest that such control over T cell subset numbers is necessary to maintain the health of the host. In an attempt to repopulate the depleted lymphocyte pool, lymphopenia is generally followed by proliferation of the remaining memory cell pool (Surh et al. Immunological Reviews. 211: 154-163), and proliferation and differentiation of naïve CD4⁺ cells to become memory CD4⁺ cells (Ernst et al. 1999. Immunity. 11: 173-181; Min et al. 2005. J. Immunol. 174: 6039-6044) by homeostatic expansion. Under conditions where the cause of lymphopenia is not removed, as is seen for memory CD4⁺ T cell loss at the onset of TID (Zhao et al. 2008. Immunology. In Press), homeostatic repopulation might not be equivalent for all immune cell subsets. This might be particularly detrimental if memory cells that secrete pro-cachexic cytokines such as, IFN-γ (Schindler et al. 1990. J. Immunol. 144: 2216-2222; Ucla et al. 1990. J. Clin. Invest. 85: 185-191), IL-1 (Cederholm et al. 1997. Am. J. Clin. Nutr. 65: 876-882; Yasumoto et al. 1995. Cancer Res. 55: 921-927; Mantovani et al. 1998. Crit. Rev. Onco. 9: 99-106), IL-6 (Cederholm et al. 1997. Am. I Clin. Nutr. 65: 876-882; Yasumoto et al. 1995. Cancer Res. 55: 921-927; Mantovani et al. 1998. Crit. Rev. Onco. 9: 99-106; Strassmann et al. 1992. J. Clin. Invest. 89: 1681-1684; Fujimoto-Ouchi et al. 1995. Int. J. Cancer. 61: 522-528), TNF-α (Dinarello et al. 1986. J. Exp. Med. 163: 1433-1450) and TGF-β (Zugmaier et al. 1991. Cancer Res. 51: 3590-3594; Chuncharunee et al. 1993. Br. J. Haematol. 84: 374-380), are preferentially activated and expanded. Alternatively, CD4⁺ CD44^{v.low} cells might inhibit cachexia by promoting the expansion of memory cells that secrete IL-4 (Sturlan et al. 2002. Anticancer Res. 22: 2547-2554) and IL-10 (Fujiki et al. 1997. Cancer Res. 57: 94-99), cytokines known for their anti-cachexic properties. However, whether cytokine manipulation is a viable treatment strategy for cachexia is currently under debate (2008. Eur. Respir. J. 31: 492-501; 2006. Am. J. Clin. Nutr. 84: 1463-1472; 2006. J. Clin. Oncol. 24: 1852-1859). It is important to note that T cells are not required for the onset of cachexia, and that cancer cachexia can be induced with similar kinetics in immunodeficient and congenic immunocompetent BALB/c recipients (Yasumoto et al. 1995. Cancer Res. 55: 921-927). However, this does not exclude the possibility that, if the immune system is present, it might play a role in enhancing the effects of cachexia. It is also possible that CD4⁺ CD44^{v.low} cells can protect from cachexia in immunodeficient recipients by a mechanism that does not involve inhibiting homeostatic expansion of memory cells.

To our knowledge this is the first report of an immune cell subset that promotes protection from cachexia, and provides a new approach in the search for novel therapeutics for the treatment of this syndrome. Whether the ability of CD4⁺ CD44^{v.low} cells to protect from cachexia reflects a novel function for CD4⁺ T cells or whether it reflects an established function that had not previously been linked specifically to CD4⁺ cells and cachexia has yet to be determined. Nevertheless, these findings provide a new insight into understanding the pathways that control the development of cachexia, and suggest a novel role for the immune system in maintaining skeletal muscle integrity.

The example below describes in greater detail some of the materials and methods used in Example 3.

### Example 4

### Mice

NOD/LtJ (NOD) and C57BL/6J adult mice were purchased from the Jackson Laboratories (Bar Harbor, Me.). All protocols used in this study were conducted according to institutional guidelines and approved by the Institutional Animal Care and Use Committee.

### Assessment of Diabetes

Every two weeks for the duration of the experiment, blood glucose levels (BGL) were tested using a one-step Bayer Glucometer Elite (Bayer, Elkhart, Ind.). Mice were considered diabetic when the BGL were >300 mg/dL over two consecutive readings.

### Lewis Lung Carcinoma Cell-Induced Cachexia

LL2 is a cell line derived from the Lewis lung carcinoma. C57BL/6 mice were injected with 5×10⁵ LL2 in the left thigh. By day 7 post-LL2 injection, a small nodule can be detected in the thigh by palpitation. Mice were terminated and tissues removed on or before day 28 post-LL2 injection, as indicated for each experiment.

### Assessment of Wasting

NOD mice were weighed once a week for the duration of the experiment. C57BL/6 mice injected with LL2 cells were weighed once a week for the first two weeks post-LL2 injection and then daily. Mice were considered wasting when their body weight was 20% less than at the beginning of the experiment. Weight loss in excess of 20% was associated with morbidity and mortality and therefore, wasting mice were sacrificed and tissues taken for analysis within 24 hours of wasting assessment, or before, as indicated in each experiment.

### Cell Purification and Transfer

Spleen cells from either 2-4 month old mice were prepared for single cell suspensions. Red blood cells were removed with lysing buffer (Sigma Chemical Co., St. Louis, Mo.), and the remaining spleen cells were resuspended in PBS with 1% Fetal Bovine Serum (Intergen Co., New York, N.Y.). Splenocytes were labeled with an APC-conjugated CD4-specific monoclonal antibody and PE-conjugated CD44-specific monoclonal antibody and CD4⁺ CD44^{v.low} cells, and CD4⁺ cells depleted of CD4⁺ CD44^{v.low} cells, were sorted under high speed on a FACSVantage SE with TurboSort (Becton Dickinson Immunocytometry Systems). The CD4⁺ CD44^{v.low} population was defined as the CD4⁺ cells that stain the weakest for CD44 and was typically 3-5% of the total CD4⁺ cells in non-diabetic NOD mice (in press), and 1-2% of the total CD4⁺cells in untreated C57BL/6 mice. In order to avoid contamination with CD4⁺ CD44^{int} cells, only the weakest staining 2% of CD4⁺ CD44^{v.low} cells in NOD and 0.8% in C57BL/6, and the brightest staining 80% of CD4⁺ cells depleted of CD4⁺ CD44^{v.low} cells were collected for the experiments described herein. All cell populations were sampled and analyzed using a FACSCalibur to confirm the purity of the sorted populations. Sorted CD4⁺ cell populations were greater than 98% CD4⁺ (data not shown). Cells were washed once in PBS after sorting and prior to intraperitoneal injection into syngeneic recipients.

### Histology and Histologic Assessment

Mice were sacrificed at the indicated times and the pancreas was removed and immediately placed in 10% neutral buffered formalin to be fixed. After 24 hours, the pancreata were embedded in paraffin and 4 μm sections were cut. Sections were tested for the presence of insulin-producing β cells by immunohistochemistry (Gu and Sarvetnick. 1993. Development. 118: 83-46). Sections were stained for insulin with guinea pig anti-porcine insulin antibody (Dako, Cartpenteria, Calif.) using the indirect immunoperoxidase method. Sections were hydrated and blocked with 10% normal goat serum (vector Laboratories, Burlinghame, Calif.). The sections were then incubated overnight in primary antibody and treated with a biotinylated secondary antibody (Vector) followed by an avidin/biotinylated enzyme complex (Vector). Slides were incubated in the dark with the enzyme substrate, 0.05% 3,3′-diaminobenzidine (Sigma chemical Co., St. Louis, Mo.) and counterstained, dehydrated and mounted with Permount (Fisher Scientific, Fairlawn, N.J.). Insulin positive areas were stained brown. The insulin positive area was accurately scored in pixels by measuring the brown coloration (insulin-positive area after insulin-specific staining) in each section of each pancreas using a Zeiss Axiovision camera and acquisition software (Carl Zeiss, inc., Thornwod, N.Y.) and KS300 analyzing system (Carl Zeiss, Inc., Thornwood, N.Y.). For each pancreas the islet area from three sections 20 mm apart was analyzed and the mean calculated.

### Skeletal Muscle Protein Isolation and Quantitation

The left and right anterior and lateral thigh muscles were isolated, weighed, then individually wrapped in autoclaved aluminum foil and stored at -80° C. until analyzed. The packed muscle was immersed in liquid nitrogen and ground with mortar and pestle. The powdered tissue was transferred into 1 ml of ice-cold homogenization buffer (Tris 0.01M, 2 mM EDTA, 0.15M NaCl, 0.012M Brij 96, 2.22 mM NP-40, 0.025 mM Leupeptin, 0.025 mM Aprotinin, 0.025 mM AEBSF) and homogenized with an electronic pellet pestle. The homogenates were incubated for 30 minutes at 4° C., and centrifuged at 14,000 g for 10 minutes at 4° C. Supernatants were thawed and diluted 1:800 in distilled H₂O on ice. Soluble protein concentration was determined by mixing 160 ml of the diluted sample with 40 ml of Bio-Rad dye reagent (Bio-RAD, Hercules, Calif.) in a 96-well plate using bovine serum albumin (BSA) as the protein standard. Using this information the weight of soluble protein in each whole muscle was calculated. Supernatant measurements were performed at least in duplicate. The plates were incubated for 10 minutes at room temperature and read at a 595 nm on a microplate reader (Molecular Devices, Sunnyvale, Calif.).

### Skeletal Muscle DNA Isolation and Quantitation

The lateral and anterior thigh muscles were excised from both hind legs of each mouse and weighed. Tissue samples (50 mg) were minced and then lysed in a 6 M guanidinium chloride buffer containing proteinase K (40 μg/ml) at 55° C. for 2-4 hours and then treated briefly with DNase-free RNase following the DNeasy protocol (Qiagen, Valencia, Calif.). Aliquots of DNA were diluted in 1M Urea for total DNA concentration measurements using a fluorometric DNA assay (Downs and Wilfinger. 1983. Anal. Biochem. 131: 538-547) (Downs and Wilfinger. 1983. Anal. Biochem. 131: 538-547) with Hoechst dye 33258 (Bio-RAD, Hercules, Calif.), and the weight of DNA in each whole muscle was calculated.

### Cell Subset Analysis

Single cell suspensions of lymph nodes (cervical, mesenteric, inguinal, para-aortic) were labeled with an allophycocyanin—(APC) conjugated CD4-specific monoclonal antibody (mAb, RM4-5) and PE-conjugated CD44-specific mAb (IM7). APC-conjugated rat IgG2a and PE-conjugated rat IgG2b were used as isotype controls. All cell populations were sampled and analyzed using a FACSCalibur with CELLQuest version 3.3 software (Becton Dickinson Immunocytometry Systems, La Jolla, Calif.) and the percentage and total number of CD4⁺ CD44^{v.low} (lowest fluorescent intensity peak for CD44 expression), CD4⁺ CD44^{int} (middle fluorescent intensity peak), and CD4⁺ CD44^{high} (highest fluorescent intensity peak) as described previously (in press) was determined. All mAbs and isotype controls were purchased from Pharmingen (La Jolla, Calif.).

### Statistical Analysis

The significance of the effect of CD4⁺ CD44^{v.low} cells on protection from cachexia on transfer into NOD recipients was assessed using the Logrank (Mantel-Cox) Test. The significance of the effect of CD4⁺ CD44^{v.low} cells on insulin secreting cells in the pancreas, the effect of LL2 cell treatment on CD4⁺ T cell lymphopenia, and the effect of CD4⁺ CD44^{v.low} cells on inhibition of CD4⁺ T cell subset lymphopenia, was determined using the Mann-Whitney Test. Skeletal muscle weight loss, and skeletal muscle protein and DNA content in cachexic animals was determined using the Student t test. A p value equal to or less than 0.05 is considered significant for all tests.

### Example 5

Since CD4⁺ CD44^{v.low} cells are preferentially lost in diabetic mice at the onset of cachexia, and can control both cancer cachexia and TID cachexia, the loss of this cell subset is likely also relevant to cachexia in humans, and that their loss in the blood of cancer patients can be used to predict and/or indicate cancer cachexia.

### CD4⁺ CD44^{v.low} Cells can be Detected in PBL of Healthy Blood Donors.

In order to accurately determine absolute numbers of CD4⁺ T cell subsets in blood, TruCount tubes (Becton Dickinson) were used that are specifically designed for this purpose. Each TruCount tube contains a defined number of beads. The absolute number of cells of interest per ml of blood is calculated using an equation that takes into account the number of beads, and the number of cells of interest, collected by FACS. For the experiment described in FIG. 17, 50 μl of blood from each of four healthy donors was aliquoted into separate TruCount tubes. A cocktail of CD4- and CD44-specific mAb in 20 μl buffer was added to the blood and incubated. 450 μl FACS lysis buffer was added, the samples were fixed (100 μl buffered fixative), mixed thoroughly to completely mix the blood and beads, and analyzed by FACS. FIG. 17A shows a representative dot plot of CD44 expression on CD4 cells. Using the number of beads in Box 1, and the number of CD4⁺ cells in Box 2, the absolute number of CD4⁺ T cells per ml of blood can be calculated. FIG. 17B shows a histogram of CD44 expression on CD4⁺ T cells. The CD4⁺ CD44^{v.low} cells were identified as the cells in the peak with the lowest mean fluorescent intensity (FIG. 17B, M1). The expression of CD44 on CD4⁺ T cells in blood did not change after overnight incubation on ice.

Using the markers shown in FIG. 17, the percentage of CD4+ CD44^{v.low} cells, as well as CD4 cells with intermediate (CD44int FIG. 17B, M2) and high (CD44high FIG. 17B, M3) expression of CD44 within the CD4+ T cell population can be calculated. The naïve, CD4+ CD44low cell subset is made up of CD4+ CD44^{v.low} plus CD4+ CD44int cells.

### Example 6

The Loss of CD4⁺ CD44^{v.low} Cells in Peripheral Blood of Cancer Patients can be used to Predict the Onset of Cachexia.

### Patient Groups.

Inclusion criteria. In the clinic, cancer cachexia is defined as the loss of 10% total body weight, loss of appetite and lethargy. The cancer patients recruited for study are those patients that have been newly diagnosed with either colon or breast cancer. Patients for the study described in this study are not cachexic at the time of initial evaluation. For this first study the patient population is limited to two cancer types in order to limit the number of variables for comparison between groups.

Exclusion criteria. Patients with any prior history of cancer are excluded from the study. In the longer term such patients might provide an additional group for study. Patients with any prior history of an immune-mediated disorder are also excluded from the study since both the disorder and the treatment are likely to affect the parameters that are critical to the study. Colon cancer patients with a growth that obstructs the colon are also excluded from the study since such an obstruction will result in weight loss. In the latter case, it is likely that such an obstruction will not be detected until after the blood has been drawn and processed. In that case the patient are removed from the study.

Control group. When possible, blood is drawn from healthy relatives/companions of the patient at the same time as the patient as this also controls for life-style of the patient. In many cases the companion is age matched but not sex matched. However, since blood is collected from men and women, the control group includes both sexes. As the study proceeds, the control group is assessed and, if sufficient numbers of age and sex matched controls have not been obtained, additional healthy individuals are included. Blood is obtained from at least 20 control subjects for this study.

### Study Design

In this study, newly diagnosed colon and breast cancer patients are monitored before, during, and after chemotherapy for the absolute number of CD4⁺ CD44^{v.low} cells, and CD4⁺ cell subsets expressing other naïve and memory cell markers, per ml of blood. Blood is drawn and analyzed by FACS (as described below in sections i) and ii)) at the time of initial evaluation (before treatment begins), and again at 2, 4, 6, 8 and 10 months post-initial evaluation (a total of six bleeds per patient). The analysis of multiple blood samples from each patient allows the determination of the variation in CD4⁺ CD44^{v.low} cell number within a single non-cachexic individual over time, as well as allowing the determination of whether the loss of this cell subset precedes cachexia, and might therefore be used to predict the onset of cachexia. Cancer therapy is different for patients with colon and breast cancer. In addition, therapy changes during the study depending on the responsiveness of the patient to the therapy. The duration of treatment also varies but is expected to be 6 months for patients without metastases and continuous for patients with metastases. Additional bleeds are taken and analyzed if clinical evaluation suggests that a patient becomes cachexic between the time points indicated. Since the absolute numbers, as well as relative percentages, of cell subsets will be calculated for each blood sample, it is not necessary to collect and analyze cachexic patient blood at the same time as blood from non-cachexic cancer patients. This allows one to collect, process and analyze cachexic and non-cachexic blood samples at different times. Because of the potential difficulty in obtaining more than one blood sample from the control subjects, a single blood sample from each is analyzed.

The goal of this study is to analyze blood from each of 20 patients with cancer cachexia. Twenty percent of the colon and breast cancer patients seen are expected to become cachexic within eight to ten months after initial evaluation. Therefore, a total of 100 patients are monitored during the two year funding period. The sample size has been calculated by statistician, and is based on data generated in the LL2 mouse model for cancer cachexia, and specifically, the number of CD4⁺ CD44^{v.low} cells in mice i) with cancer cachexia compared to ii) mice with cancer without cachexia and iii) mice without cancer. Based on these data, the number of cachexic patients in the study (n=20) ensures a power of 0.90 to detect a moderate effect size of 0.4 with a standard analysis of variance procedure at conventional alpha level 0.05.

### i) Lymphocyte Cell Subset Evaluation

The expression of CD44, CD45RB and CD45RO on CD4⁺ T cells in the blood of cancer patients and control subjects is analyzed by FACS as follows:

CD44. On arrival 50 μl of whole blood is aliquoted into TruCount tubes (Becton Dickinson, Calif.). Blood is mixed with 20 μl fluorochrome-conjugated mAb specific for CD4 and CD44. The numbers and percentages of CD4⁺ T cells that express CD44^{v.low}, CD44^{low}, CD44^{int} and CD44^{high} in the blood is determined by FACS as described in the Preliminary Data section. Isotype controls for the CD4-, and CD44-specific mAbs are included for each sample.

CD45RA and CD45RO. In the mouse, the level of expression of CD44 is the best marker to distinguish naïve from memory cells (25-26). However, in human, the two markers that distinguish naïve from memory cells are CD45RA and CD45RO. Human CD4⁺ T cells that are naive express CD45RA but not CD45RO (29), and memory CD4⁺ T cells express CD45RO but not CD45RA (30-32). Since it is not known whether the function of CD4⁺ CD44^{v.low} cells is related to the fact that they are a subset of naïve CD4⁺ T cells, or whether the function is dependent on a very low expression of CD44, the expression of CD45RA and CD45RO on CD4⁺ T cells of cancer patients is also determined. Therefore, blood is also labeled with fluorochrome-conjugated mAbs specific for CD4 and either CD45RA or, CD45RO, and the expression of these markers on CD4⁺ T cells is determined by FACS.

### ii) Apoptosis and Proliferation.

In addition to the CD4⁺ cell subset analysis the hypothesis that a greater percentage of CD4⁺ CD44^{v.low} cells undergo apoptosis in cancer patients with cachexia than they do in cancer patients that are not cachexic is tested. The alternative hypothesis that a lower percentage of CD4⁺ CD44^{v.low} cells undergo proliferation in cachexic patients compared to non-cachexic patients is also tested. In order to determine the number and percentage of CD4⁺ T cell subsets that are proliferating and undergoing apoptosis, whole blood is labeled first with either, the DNA dye, Hoechst 33342 (Molecular Probes), or, with PE-conjugated annexin V. This is followed by cell surface labeling with CD4- and either CD44, or CD45RA, or CD45RO-specific mAb, and FACS analysis. An increase in the intensity of Hoechst label indicates that the cell is undergoing proliferation, while annexin V positive cells are those that are undergoing apoptosis. All labeling is performed in TruCount tubes.

iii) Further Phenotype.

It was shown that, in the NOD mouse strain, CD4⁺ CD44^{v.low} cells express CD3, CD62L^{high} and an intermediate and high density of CD45RB, consistent with a naïve cell phenotype (10). They do not express the activation/regulatory markers CD25 and CD38 (10). CD4⁺ CD44^{v.low} cells also do not express the regulatory marker Foxp3. Using blood taken from five cancer patients at the time of initial evaluation, and five control subjects, it is determined whether CD4⁺ CD44^{v.low} cells in non-cachexic cancer patients and control subjects have the same phenotype as they do in mice by co-labeling CD4⁺cells with monoclonal antibody (mAb) specific for CD44 and either CD3, or, CD62L, or, CD45RB, or CD25, or, CD38, or Foxp3. It is also determined whether CD4⁺ CD44^{v.low} cells express the naïve phenotype, CD45RA⁺, by co-labeling cells with CD4-, CD44- and CD45RA-specific mAbs. All of these mAbs are available from Pharmingen (La Jolla, Calif.). The percentage of CD4⁺ CD45RA⁺ cells that expresses CD44^{v.low} is also determined.

### Predicted Results and Interpretation:

If it is found that the number and percentage of CD4⁺ CD44^{v.low} cells in the blood of non-cachexic cancer patients is greater than in cancer patients that are cachexic, and that the percentage and number of CD4⁺ CD44^{v.low} cells is lower in patients with cachexia than in the same patients before cachexia developed, the loss of CD4⁻ CD44^{v.low} cells is associated with cachexia. Data from age and sex matched cachexic and non-cachexic patients with the same cancer type and treatment regimen is compared throughout the study. In light of the fact that the number of cachexic patients analyzed will be in the order of 20, data from all cachexic patients is also compared to data from all non-cachexic patients. If found that cancer patients that become cachexic lose CD4⁺ CD44^{v.low} cells in the blood before they develop cachexia, this suggests that the loss of this cell subset might be used to predict the onset of cachexia. Any relationship between the degree of CD4⁺ CD44^{v.low} cell loss and the severity of cachexia is also determined. CD44 is the relevant marker for the association between cachexia and naïve CD4⁺ T cell loss and not CD45RB. However, this is formally tested in this study. With respect to the hypothesis that cancer cachexia is associated with the loss of CD4⁺ CD44^{v.low} cells, since the mouse data show a role for this cell subset in cachexia induced by two different primary diseases, namely, type I diabetes and cancer, the association is also likely to exist in human disease.

If found that the absolute number and/or percentage of CD4⁺ CD44′^{v.low} cells is greater in control subjects than in cancer patients without cachexia this suggests that either cancer itself, or cancer with chemotherapy, plays a role the loss of this cell subset.

If the data show that a loss of CD4⁺ CD44^{v.low} cells in patients with cancer cachexia is associated with an increase in apoptosis of CD4⁺ CD44^{v.low} cells, and not a decrease in proliferation, this suggests that CD4⁺ CD44^{v.low} cell lymphopenia is the result of cell death.

Patients with cancer cachexia are often lymphopenic and therefore cancer patients with cancer cachexia show a loss in both naïve (CD45RB⁺) and memory (CD45RO⁺) CD4⁺ T cells compared to patients with cancer that are not cachexic. However, the loss of CD4⁺ CD44^{v.low} cells precedes, and is more extensive than, the loss of the naïve and memory cell subsets. The effect of cancer and cancer cachexia on CD4⁺ CD44^{int} and CD4⁻ CD44^{high} cells is also be evaluated, and the loss of these cell subsets is gradual as disease progresses, but is specific to cachexia.

The phenotype of CD4⁺ CD44^{v.low} cells in human cancer patients without cachexia is the same as that seen in the mouse.

Since the treatment regime given to each colon and breast cancer patient is selected based on the responsiveness of the patient to therapy, for full analysis the cachexic and non-cachexic patient groups are divided into sub-groups to control for treatment, as well as cancer type, and age and sex. However, despite this variation, data is sufficiently clear.

### Example 7

CD4⁺ CD44^{v.low} Cells in Peripheral Blood can be used as a Biomarker to Indicate Cachexia in Cancer Patients that have never been Treated for Cancer.

On rare occasions (3-5 patients a year) cancer patients that have not previously been treated for cancer present with cachexia at their initial evaluation. These patients provide an opportunity to evaluate the effect of cachexia on immune parameters in cancer patients in the absence of cancer treatment. In this study, the expression of CD44, as well as other naïve and memory cell markers, on CD4⁺ T cells from previously untreated cancer patients that already have cachexia, is compared with the CD4⁺T cells from non-cachexic cancer patients that are newly diagnosed with the same cancer.

### Patient Groups.

Inclusion criteria. Two groups of cancer patients are recruited for the study outlined in this study. One group is those patients newly diagnosed with cancer that are already cachexic at initial evaluation, and the second group is those patients newly diagnosed with cancer that are not cachexic at initial evaluation. Since this patient population is so rare, all cancer types are evaluated, except for those described in the exclusion criteria section.

Exclusion criteria. The exclusion criteria are the same as those for Example 6.

Control group. The control group is selected using the same criteria as described for Example 6.

### Study Design

In this study, a single blood sample was analyzed taken at initial evaluation from newly diagnosed cancer patients that are cachexic, and newly diagnosed cancer patients that are not cachexic. As described in Example 6, the absolute number of CD4⁺ CD44^{v.low} cells, as well as CD4⁺ CD44^{int}, CD4⁺ CD44^{high} CD4⁺ CD45RB⁺, and CD4⁺ CD45RO⁺ cells per ml of blood is determined for each sample.

All other aspects of study design in this study, including lymphocyte subset analysis, and analysis of CD4⁺ T cell apoptosis and proliferation, are the same as those described for Example 6.

### Predicted Results and Interpretation

If the blood of patients with cancer cachexia contain fewer CD4⁺ CD44^{v.low} cells that cancer patients that are not cachexic, the loss of CD4⁻ CD44^{v.low} cells in the blood might indeed be used as a marker to indicate cachexia in cancer patients that have not received therapy for cancer.

Data in this study also indicate to us the nature of the CD4⁺ T cell lymphopenia seen in cachexic patients, and whether it dominantly affects naïve or memory cells. All CD4⁺ T cell subsets are reduced in the blood of cachexic patients but, at the onset of cachexia, the cell subset that is affected first is the CD4⁺ CD44^{v.low} cells population.

The goal of this study is very important with respect to comparison with mouse models of cachexia, because in the latter models, CD44 expression on CD4⁺ T cells is evaluated in mice that had not received any therapy. In addition, if cancer cachexia in patients treated with chemotherapy is not associated with a loss of CD4⁺ CD44^{v.low} cells in Example 6, this study helps address whether the lack of an association is due to differences between human and mouse disease, or whether it relates to the treatment given to cancer patients.

### Long Term.

The loss of CD4⁺ CD44^{v.low} cells can be used as a biomarker to indicate, and predict, the onset of cachexia. The presence of CD4⁺ CD44^{v.low} cells in the blood of cancer patients that are not cachexic correlates with a period during which therapeutic approaches are feasible. The CD4⁺ CD44^{v.low} cell subset can be used to monitor the success of therapeutic intervention. Finally, this study shows there is a correlation between the severity of the immune cell subset imbalance and the kinetics and severity of cachexia progression. This is useful in the development of novel therapeutic strategies for the treatment of cachexia in cancer patients, and possibly patients with other chronic diseases.

### Example 8

CD4⁺ CD44^{v.low} Cells in the PBL of Patients with TID can be used as a Biomarker to Indicate the Onset (Increase in CD4⁺ CD44^{v.low} Cells) and Loss (Decrease CD4⁺ CD44^{v.low} Cells) of the Honeymoon Period

The honeymoon period in Type I Diabetes (TID) is the transient partial remission seen primarily in children with new onset TID (1). Partial remission is thought to be due to an increase in β-cell mass (2), or, in some patients, to a transient resolution in insulin resistance (3), leading to a period with low insulin requirement. Currently there is no biomarker that can accurately identify the honeymoon period. The goal of this study is to identify a biomarker in the blood of TID patients that will indicate both the onset, and the loss, of the honeymoon period.

In the absence of exogenous insulin treatment, diabetic NOD mice (the mouse model for spontaneous TID), experience severe weight loss (wasting) and muscle atrophy within 2-6 weeks of diabetes onset (4). CD4⁺ T cells that express a very low density of CD44 (a small peak of CD4⁻ T cells that express the lowest density of CD44), but not CD4⁺ T cells that express either an intermediate or a high density of CD44, are significantly lost in diabetic mice that are wasting, but not in diabetic mice that are not yet wasting. The loss of CD4⁺ CD44^{v.low} cells is also associated with the inability of diabetic mice to respond to low doses of insulin (Preliminary Data), while diabetic mice that do respond to low doses of insulin have a number of CD4⁺ CD44^{v.low} cells equivalent to that in non-diabetic mice (Preliminary Data). Moreover, our published data show that CD4⁺ CD44^{v.low} cells delay the onset of wasting in diabetic NOD mice, and promote a significant increase in β-cell mass when infused into NOD. SCID mice (Preliminary Data). These data strongly suggest an association between CD4⁺ CD44^{v.low} cells and the honeymoon period in TID. This study was designed to address the primary hypothesis that an increase in CD4⁺ CD44^{v.low} cells in the PBL of patients with TID reflects the onset of the honeymoon period, while a loss of the same cell subsets reflects the loss of the honeymoon period.

### b) Background and Significance

Autoimmune destruction of pancreatic β-cells results in TID with low insulin production and high blood glucose levels (BGL) (5-6). Insulin resistance can also play a role in the overall increase in BGL in patients with TID, but to a lesser extent (7-8). Soon after the diagnosis of TID, many children, particularly those between the ages of 7 and 16 years, experience a period of partial remission characterized by the ability of a low dose of exogenous insulin to achieve euglycemia (1, 9). This period is termed, the honeymoon period, and can last from days to months (10). Clinically the honeymoon period is defined as a daily insulin requirement of less than 0.5 U/kg/day (11-13). The mechanisms that lead to the onset of the honeymoon period involve both an increase in insulin-secreting β-cell mass (2), and a resolution of insulin resistance (3). β-Cell mass increases in response to a variety of conditions that result in metabolic changes (14), including moderate hyperglycemia (15). The increased demand for insulin caused by insulin resistance can also lead to an increase in β-cell mass (16), and the resolution of insulin resistance can then promote the onset of the honeymoon period. Insulin therapy has also been implicated in promoting the onset of the honeymoon period by ameliorating β-cell destruction (11). However, this does not explain the loss of the honeymoon period with continued insulin therapy. As the autoimmune response continues to destroy the pancreatic β-cells, glucose levels increase leading to further β-cell damage (15) and, the loss of the honeymoon period.

The non-obese diabetic (NOD) mouse strain is the well-characterized mouse model for spontaneous TID (17-18). Within 2-6 weeks after the onset of diabetes, in the absence of exogenous insulin treatment, diabetic NOD mice become wasting (4), and wasting is associated with a further loss of β-cell mass. These data have led us to speculate that wasting in the NOD mouse might be related to the loss of the honeymoon period. In human TID, weight loss is often evident in patients before TID diagnosis. However, after insulin treatment, wasting is reversed and the honeymoon period begins. In this case the data might suggest an association between reversal of wasting and the onset of the honeymoon period. However, there is a paradox in that the loss of the honeymoon period in TID patients is not associated with wasting. This paradox might be explained by the fact that TID patients continue to take insulin after the loss of the honeymoon period, and insulin treatment reverses weight loss and promotes growth in TID patients (19-20). Therefore, TID patients do not lose weight when the honeymoon period is lost, because they are treated with exogenous insulin.

CD44 is one of the well-established cell surface markers used to distinguish antigen inexperienced (naïve, CD44^{low}) from antigen experienced (memory, CD44^{high}) CD4⁺ T cells in the mouse. Thus, naive CD4⁺ T cells express CD44^{low} and a high density of CD62L (CD62L^{high}) while memory cells express CD44 at a high density (CD44^{high}) (21-22). A subset of CD4⁺ CD44^{low} cells, defined by their expression of the lowest density of CD44 (CD4⁺ CD44^{v.low}), but not CD4⁺ cells that express either an intermediate or a high density of CD44, are preferentially depleted in diabetic mice that are wasting, but not in diabetic mice that are not wasting (4). In addition, diabetic mice that are depleted of CD4⁺ CD44^{v.low} cells are not responsive to low doses of insulin, whereas those diabetic mice that are not depleted of CD4⁺ CD44^{v.low} cells become euglycemic in response to a low dose of insulin. Furthermore, injecting diabetic mice with highly purified CD4⁺ CD44^{v.low} cells sorted from pre-diabetic NOD mice can delay the onset of wasting (23), and that they promote a significant increase in insulin-secreting β-cell mass on transfer into NOD. SCID recipients, suggesting that CD4⁺ CD44^{v.low} cells might play a causal role in the progression of disease. In this application, the hypothesis that will be tested is CD4⁺ CD44^{v.low} cells in the blood of patients with TID can be used as a biomarker to indicate the onset (increase in CD4⁺ CD44^{v.low} cells) and loss (decrease CD4⁺ CD44^{v.low} cells) of the honeymoon period.

Significance to TID in human: Treatment of new onset TID patients with anti-CD3 monoclonal antibody (mAb) leads to an increase in residual β-cell function, compared to a placebo treated group, at 18 months post-treatment (24-26). Moreover, treatment was most effective in those patients with the highest residual β-cell function at the time of treatment, ie. during the honeymoon period. Therefore, identifying biomarkers that can accurately identify the honeymoon period is likely to be extremely important in identifying patients who are most likely to respond to treatments aimed at reversing TID. Identifying strategies that delay the loss of the honeymoon period is also highly significant.

### c) Preliminary Data

The number of CD4⁺ CD44^{v.low} Cells in the Blood of Diabetic NOD Mice Correlates with Insulin Requirement

NOD mice that were either diabetic but not wasting (n=7), or diabetic and wasting (n=8), or non-diabetic (n=5), were injected with a low dose of insulin subcutaneously. BGL was taken immediately before and one hour after insulin injection. After the second BGL measurement, all mice were bled and the absolute number of CD4⁺ CD44^{v.low} cells per ml of blood was determined by FACS using TruCount tubes as described above.

TABLE 4
Correlation between response to insulin, CD4⁺ CD44^{v.low} cell number
and wasting

| | | | | |
|---|---|---|---|---|
| | BGL | BGL | CD4⁺ CD44^{v.low} | |
| | pre-insulin | post-insulin | number per | weight |
| Mouse # | (mg/dL) | (mg/dL) | ml blood | (g) |

a. Diabetic mice that respond to insulin

| | | | | |
|---|---|---|---|---|
| 1 | >600 | 103 | 730 | 22 |
| 2 | 376 | 44 | 2,810 | 24 |
| 3 | 576 | 146 | 584 | 24 |
| 4 | >600 | 176 | 1,675 | 26 |
| 5 | >600 | 107 | 509 | 24 |
| 6 | 385 | 98 | 1,822 | 25 |
| 7 | 359 | 64 | 1,543 | 27 |

b. Diabetic mice that do not respond to insulin

| | | | | |
|---|---|---|---|---|
| 1 | <600 | >600 | 48 | 24 |
| 2 | >600 | >600 | 116 | 18 |
| 3 | >600 | >600 | 42 | 18 |
| 4 | >600 | >600 | 40 | 20 |
| 5 | >600 | 572 | 90 | 21 |
| 6 | >600 | >600 | 190 | 19 |
| 7 | >600 | >600 | 180 | 20 |
| 8 | >600 | >600 | 100 | 17 |

c. Non-diabetic mice

| | | | | |
|---|---|---|---|---|
| 1 | 124 | 48 | 1,150 | 25 |
| 2 | 94 | 33 | 789 | 24 |
| 3 | 280 | 55 | 1,853 | 26 |
| 4 | 98 | 36 | 1,870 | 23 |
| 5 | 109 | 30 | 2,200 | 24 |

All diabetic mice that responded to the low dose of insulin by becoming euglycemic contained greater than 500 CD4⁺ CD44^{v.low} cells per ml of blood and none were wasting (Table 4a). In contrast, all of the diabetic mice that did not respond to insulin contained fewer than 200 CD4⁺ CD44^{v.low} cells per ml blood and all but one were wasting (Table 4b). All non-diabetic mice responded to insulin and all contained greater than 500 CD4⁺ CD44^{v.low} cells per ml blood (Table 4c). In general, mice that had a lower BGL before insulin injection were more responsive to the insulin. These data strongly support the hypothesis that the number of CD4⁺ CD44^{v.low} cells in the blood can indicate responsiveness to insulin, and might be used to indicate the onset and loss of the honeymoon period.

CD4⁺ CD44^{v.low} Cells Promote an Increase in Insulin Secreting β-Cell Mass on Transfer into NOD.SCID Mice

NOD.SCID recipient mice were injected with either 1×10⁵ CD4⁺ CD44^{v.low} cells, or an equal number of CD4⁺ cells depleted of CD44^{v.low} cells, sorted from 12 week old pre-diabetic female NOD donors. An additional group of recipients was left without a cell infusion. Mice were monitored for diabetes by measuring BGL every week. Four weeks after cell infusion all mice were sacrificed, and the pancreas was removed and analyzed for the presence of insulin by immunohistochemistry. The relative amount of insulin in each pancreas was measured in pixels using standard published methods as previously described by us, and others (24). Briefly, the pancreas is immediately placed in 10% neutral buffered formalin to be fixed. After 24 hours, the pancreata are embedded in paraffin and 4 μm sections cut. Sections are stained for insulin with guinea pig anti-porcine insulin antibody (Dako, Cartpenteria, Calif.) using the indirect immunoperoxidase method. Sections are hydrated and blocked with 10% normal goat serum (vector Laboratories, Burlinghame, Calif.). The sections are then incubated overnight in primary antibody and treated with a biotinylated secondary antibody (Vector) followed by an avidin/biotinylated enzyme complex (Vector). Slides are incubated in the dark with the enzyme substrate, 0.05% 3,3′-diaminobenzidine (Sigma chemical Co., St. Louis, Mo.) and counterstained, dehydrated and mounted with Permount (Fisher Scientific, Fairlawn, N.J.). Insulin positive areas are stained brown. The insulin positive area is accurately scored in pixels by measuring the brown coloration (insulin-positive area after insulin-specific staining) in each section of each pancreas using a Zeiss Axiovision camera and acquisition software (Carl Zeiss, inc., Thornwod, N.Y.) and KS300 analyzing system (Carl Zeiss, Inc., Thornwood, N.Y.). For each pancreas the islet area from three sections 20₁Am apart is analyzed and the mean calculated.

All of the mice in the group that received CD4⁺cells depleted of CD44^{v.low} cells were diabetic by the time they were sacrificed, probably due to the presence of autoreactive memory cells within the transferred population. Not surprisingly, the insulin positive area in this diabetic group was very low compared to the group of mice that did not receive a cell infusion (Table 5). In contrast, none of the mice in the group that received CD4+ CD44^{v.low} cells were diabetic by this time point, suggesting that either this cell subset does not contain autoreactive cells, or that the frequency of autoreactive cells is too low to cause diabetes within this time frame. Moreover, the amount of insulin measured in the group of mice that received CD4⁺ CD44^{v.low} cells was significantly greater than the group that received no cells (p<0.03, Student t test) indicating that CD4⁺ CD44^{v.low} cells promoted an increase in insulin positive β-cell area in NOD. SCID mice (Table 5). The mechanism of this increase in insulin positive (3-cell mass is currently under investigation. These data are shown to indicate a potential mechanism by which CD4⁺ CD44^{v.low} cells might promote the honeymoon period in TID, and this is also currently under investigation, but not part of this proposal. Data are shown as mean±SEM of insulin-positive pixels in all pancreata within each group.

TABLE 5
CD4⁺ CD44^{v.low} cells promote an increase
in insulin secreting β-cell mass

| Treatment | |
|---|---|
| (pixels) | Area of insulin secreting β-cells |
| CD4⁺ CD44^{v.low} (n = 4) | 171,110 +/− 37,826 |
| CD4⁺ depleted of CD44^{v.low} (n = 5) | 911 +/− 792 |
| No cells (n = 5) | 60,407 +/− 21,763 |

### d) Research Design and Methods

Patient Group (n=40)

Inclusion criteria:

1) Initial bleed—Symptoms of hyperglycemia (polyuria, polydipsia, or unexplained weight loss), with a blood sugar level greater than, or equal to, 200 mg/dL (2005 American Diabetes Association diagnosis criteria). Second bleed—evidence of onset of honeymoon period based on low insulin requirement. Third bleed—Evidence of loss of the honeymoon period based on increase in insulin requirement. This is a longitudinal study. The same patients are followed for all three bleeds.

2) Age 7-16 years old.

Exclusion criteria: Patients with any prior history of an immune-mediated disorder are excluded from the study since both the disorder and the treatment are likely to affect the parameters that are critical to the study.

Control group A (n=40)

Age matched subjects who attend the clinic for growth evaluation and who have no history, or family history, of TID. As with the patient group above, any individuals with any prior history of an immune-mediated disorder are excluded from the study since both the disorder and the treatment are likely to affect the parameters that are critical to the study. These subjects are bled one time only, when they attend the clinic for their first evaluation.

Control group B (n=40)

Age matched subjects who attend the clinic with high blood glucose levels but who are diagnosed with type II diabetes and not type I diabetes. As with the groups above, any individuals with any prior history of an immune-mediated disorder are excluded from the study since both the disorder and the treatment are likely to affect the parameters that are critical to the study. These patients are bled one time only, on their first visit to the clinic for their initial evaluation. This control group provides age matched controls for the effect of hyperglycemia on the CD4⁺ CD44^{v.low} cells.

Patients and control subjects are recruited. Control subjects and patients who fit the inclusion criteria will be identified, and then they and their families will be approached to discuss consent. Blood is analyzed from each of 40 TID patients, 40 age matched control subjects who attend the clinic for growth evaluation, and 40 age matched control subjects newly diagnosed with type II diabetes. The sample size has been calculated by a statistician, and is based on data generated in the NOD mouse model for TID (number of CD4⁺ CD44^{v.low} cells in PBL in i) pre-diabetic mice compared to ii) mice with TID before wasting and iii) mice with TID and wasting). Based on these data, the number of patients in each group ensures a power of 0.90 to detect a moderate effect size of 0.4 with a standard analysis of variance procedure at conventional alpha level 0.05. This number was then doubled to accommodate the additional variation in the human population compared to an inbred mouse population.

For this study, TID patients are bled three times, once when they attend the clinic for their initial evaluation and before insulin treatment, a second time at the onset of the honeymoon period, as determined by Dr. Gottschalk (when the insulin requirement for the patient is decreased), and a third time at the loss of the honeymoon period, again as determined by Dr. Gottschalk (when the insulin requirement increases again). In the Rady Chlidrens Hospital diabetes clinic more than 75% of children diagnosed with TID experience a honeymoon period and all of those do so within six weeks of diagnosis (Gottschalk, unpublished observations). For this study, children who do not enter a honeymoon period within six weeks of diagnosis are omitted from the study. Control subjects are bled one time only, at their first visit to the clinic.

### Time Line (Table 6):

Table 6 shows a timeline for collection and analysis of blood samples from TID and control subjects.

TABLE 6
A schematic to show the time line for collection and analysis
of blood samples from TID and control subjects

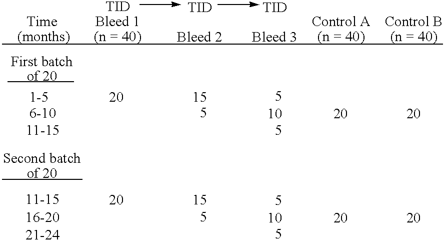

For the majority of patients, the honeymoon period is lost within a year of diagnosis. Since the absolute numbers, as well as relative percentages, of cell subsets are calculated for each blood sample, it is not necessary to collect and analyze control subject blood at the same time as blood from TID patients. This allows us to collect a single blood sample from 20 control subjects rather than three samples from each. The blood is immediately processed and analyzed by FACS.

### i) Lymphocyte Cell Subset Evaluation.

CD44. On arrival 50 μl of whole blood is aliquoted into TruCount tubes (Becton Dickinson, Calif.). Blood is mixed with 20 μl fluorochrome-conjugated mAb specific for CD4 and CD44. The numbers and percentages of CD4⁺ T cells that express CD44^{v.low} CD44^{low}, CD44^{int} and CD44^{high} in the blood is determined by FACS as described in the Preliminary Data section. Isotype controls for the CD4-, and CD44-specific mAbs are included for each sample.

CD45RA and CD45RO. In the mouse, the level of expression of CD44 is the best marker to distinguish naïve from memory cells (21-22). However, in human, the two markers that distinguish naïve from memory cells are CD45RA and CD45RO. Human CD4⁺ T cells that are naive express CD45RA but not CD45RO (27), and memory CD4⁺ T cells express CD45RO but not CD45RA (28-30). Since it is not known whether the function of CD4⁺ CD44^{v.low} cells is related to the fact that they are a subset of naïve CD4⁺ T cells, or whether the function is dependent on a very low expression of CD44, the expression of CD45RA and CD45RO on CD4⁺ T cells of TID and control subjects is also determined. Therefore, blood from patient and control subjects is also labeled with fluorochrome-conjugated mAbs specific for CD4 and either CD45RA or, CD45RO, and the expression of these markers on CD4⁺ T cells is determined by FACS.

### ii) Apoptosis and Proliferation

In addition to the cell subset analysis the hypothesis is tested that a greater percentage of CD4⁺ CD44^{v.low} cells are proliferating in TID patients at the onset of the honeymoon period compared to TID patients either before the onset of the honeymoon period, or after the loss of the honeymoon period. Similarly, the hypothesis is tested that a greater percentage of CD4⁺ CD44^{v.low} cells are undergoing apoptosis in TID patients at the loss of the honeymoon period compared to either, TID patients at the onset of the honeymoon period, or control individuals. In order to determine the number and percentage of CD4⁺ T cell subsets that are proliferating and undergoing apoptosis, whole blood is labeled first with either, the DNA dye, Hoechst 33342 (Molecular Probes), or, with PE-conjugated annexin V. This will be followed by cell surface labeling with CD4- and either CD44, or CD45RA, or CD45RO-specific mAb, and FACS analysis. An increase in the intensity of Hoechst label indicates that the cell is undergoing proliferation, while annexin V positive cells are those that are undergoing apoptosis. All labeling is performed in TruCount tubes.

iii) Further Phenotype

In the NOD mouse strain, CD4⁺ CD44^{v.low} cells expressed CD3, CD62L^{high} and an intermediate and high density of CD45RB, consistent with a naïve cell phenotype (4). They do not express the activation/regulatory markers CD25 and CD38 (4). CD4⁺ CD44^{v.low} cells also do not express the regulatory marker Foxp3. Using five subjects from each of the two control groups and five TID patients at the onset of the honeymoon period, it is determined whether CD4⁻ CD44^{v.low} cells in human have the same phenotype as they do in mice by co-labeling CD4⁺ cells with monoclonal antibody (mAb) specific for CD44 and either CD3, or, CD62L, or, CD45RB, or CD25, or, CD38. It is also determined whether CD4⁺ CD44^{v.low} cells express the naïve phenotype, CD45RA⁺, by co-labeling cells with CD4-, CD44- and CD45RA-specific mAbs. All of these mAbs are available from Pharmingen (La Jolla, Calif.). The percentage of CD4⁺ CD45RA⁺ cells that expresses CD44^{v.low} will be be determined.

### Predicted Results and Interpretation

The phenotype of CD4+ CD44^{v.low} cells in human TID, TIID or in non-diabetic control subjects is the same as that seen in the mouse. Blood from patients after the onset of the honeymoon period, shows an increase in the total number and percentage of CD4+ CD44^{v.low} cells and CD4+ CD45RA+ cells, compared to blood from the same TID patients taken either at their first visit to the clinic before insulin treatment, or after the loss of the honeymoon period. The number and percentage of CD4+ CD44^{v.low} cells in the blood of control subjects is greater than in TID patients after the loss of the honeymoon period and either equal to, or less than, that in TID patients during the honeymoon period. Further, the increase in CD4+ CD44^{v.low} cells during the honeymoon period is demonstrated as due to an increase in proliferation whereas the loss of the honeymoon period is associated with an increase in apoptosis of the same cell subset.

The effect of hyperglycemia on the CD4⁺ CD44^{v.low} cell population is difficult to predict in TID versus TIID patients. Based on an association between CD4⁺ CD44^{v.low} cells and the onset and loss of the honeymoon period, and a correlation between the CD4⁺ CD44^{v.low} cell population in the first bleed in TID and TIID patients, a follow up study is performed to demonstrate that the CD4⁺ CD44^{v.low} cell population is the same when hyperglycemia in resolved in TIID compared to the first bleed in the same patient group at initial evaluation.

### Long Term

Follow up studies are performed to demonstrate that the increase in CD4⁺ CD44^{v.low} cells in the blood at the onset of the honeymoon period correlates with a period during which therapeutic approaches are feasible. These studies show the CD4⁺ CD44^{v.low} cell subset can be used to monitor the success of therapeutic intervention.

Based on validation of the CD4⁺ CD44^{v.low} cell population as a biomarker for the onset and loss of the honeymoon period in patients with TID, the same immune cell subset is used to identify the onset and loss of the honeymoon period in the NOD mouse, and to elucidate mechanistic insight into the basis of the honeymoon period.

### E) LITERATURE CITED

1. Abdul-Rasoul, M., Habib, H., and Al-Khouli, M. 2006. ‘The honeymoon phase’ in children with type I diabetes mellitus: frequency, duration, and influential factors. Pediatr. Diabetes. 7:101-107.
2. Lombardo, F., Velenzise, M., Wasniewska, M., Messina, M. F., Ruggeri, C., Arrigo, T., and De Luca, F. 2002. Two-year prospective evaluation of the factors affecting honeymoon frequency and duration in children with insulin dependent diabetes mellitus: the key-role of age at diagnosis. Diabetes Nutr. Metab. 15:246-251.
3. Greenbaum, C. J. 2002. Insulin resistance in type I diabetes. Diabetes metab. Res. Rev. 18:192-200.
4. Zhao, C., Wang, Z., Robertson, M. W., and Davies, J. D. 2008. Cachexia in the non obese diabetic mouse is associated with CD4+ T cell lymphopenia. Immunology. In Press.
5. Castano, L., and G. S. Eisenbarth. 1990. Type-I diabetes: a chronic autoimmune disease of human, mouse and rat Annu. Rev. Immunol. 8: 647-679.
6. Tisch, R., and H. McDevitt. 1996. Insulin-dependent diabetes mellitus. Cell 85: 291-297.
7. Ginsberg, H. N. 1977. Investigation of insulin sensitivity in treated subjects with ketosis-prone diabetes mellitus. Diabetes. 26: 278-283.
8. Leslie, R. D., Taylor, R., and Pozzilli, P. 1997. The role of insulin resistance in the natural history of type I diabetes. Diabet. Med. 14:327-331.
9. Keymeulen, B., and Somer, G. 1993. Immunointervention in type-I (insulin-dependent) diabetes mellitus. Acta Clinica Belg. 48: 86-95.
10. Madsbad, S., Krarup, T., Regeur, L., Faber, O. K., and Binder, C. 1980. Insulin secretory reserve in insulin dependent patients at time of diagnosis and the first 180 days of insulin treatment. Acta Endocrinol (Copenh) 95:359-363.
11. Muhammad, B. H., Swift, P. G. F., Raymond, N. T., and Botha, J. L. 1999. Partial remission phase of diabetes in children younger than 10 years. Arc. Dis. Child. 80: 367-369.
12. Bober, E., Dunbar, B., and Buyukgebiz A. 2001. Partial remission phase and metabolic control in type I diabetes meelitus in children and adolescents. J. Pediatr. Endocrinol. Metab. 14: 435-441.
13. Buyukgebiz, A., Cemeroglu, A. P., Bober, E., Mohn, A., and Chiarelli, F. 2001. Factors influencing remission phase in children with type I diabetes meelitus. J. Periatr. Endocrin. Metab. 14: 1585-1596.
14. Bruning, J. C., Winnay, J., Bonner-Weir, S., Taylor, S. I., Accili, D., and Kahn, R. C. 1997. Development of a novel polygenic model of NIDDM in mice heterozygous for IR and IRS-1 null alleles. Cell. 88:561-572.
15. Maedler, K., Fontana, A., Ris, F., Sergeev, P., Toso, C., Oberholzer, J., Lehmann, R., Bachman, F., Tasinato, A., Spinas, G. A., Halban, A., Donath, M. Y. 2002. FLIP switches Fas-mediated glucose signaling in human pancreatic beta cells from apoptosis to cell replication. Proc. Natl. Acad. Sci. USA. 99:8236-8241.
16. Weir, C. G., Laybutt, D. R., Kaneto, H., Bonner-Weir, S., Sharma, A. 2001. b-Cell adaptation and decompensation during the progression of diabetes. Diabetes 50:154-159.
17. Kikutani H, and Makino S. The murine autoimmune diabetes model: NOD and related strains. Adv Immunol 1992; 51:285-322.
18. Makino S, Kunimoto K, Muraoka Y, Mizushima Y, Katagiri K, and Tochino Y. Breeding of a non-obese, diabetic strain of mice. Jikken Dobutsu 1980; 29:1-213.
19. Nair K S, Ford G C, Ekberg K, Fernqvist-Forbes E, Wahren, J. Protein dynamics in whole body and in splanchnic and leg tissues in type I diabetic patients. J Clin Invest 1995; 95:2926-37.
20. Kettelhut, I. C., Wing, S. S., and Goldberg, A. L. 1988. Endocrine regulation of protein breakdown in skeletal muscle. Diabetes Metab. Rev. 4:751-772.
21. Budd, R. C., Cerottini, J. C., Horvath, C., Bron, C., Pedrazzini, T., Howe, R. C., and MacDonald, H. R. 1987. Distinction of virgin and memory T lymphocytes: stable acquisition of the Pgp-1 glycoprotein concomitant with antigenic stimulation. J. Immunol. 138:3120-3129.
22. Swain, S. L. 1994. Generation and in vivo persistence of polarized Th1 and Th2 memory cells. Immunity 1:543-552.
23. Wang, A., Zhao, C., Davies, J. D. 2008. A novel role for CD4+ T cells in the control of cachexia. J. Immunol. In Press.
24. Keymeulen, B., Vandemeulebroucke, E., Ziegler, A. G., et al. 2005. Insulin needs after CD3-antibody therapy in new-onset type I diabetes. N. Eng. J. Med. 352:2598-2608.
25. Herold, K. C., Hagopian, W., Auger, J. A., et al. 2005. Anti-CD3 monoclonal antibody in new-onset type I diabetes mellitus. N. Engl. J. Med. 346:1692-1698.
26. Herold, K. C., Gitelman, S. E., Masharani, U., et al. 2005. A single course of anti-CD3 monoclonal antibody hOKT3g1(Ala-Ala) results in improvement in C-peptide responses and clinical parameters for at least 2 years after onset of type I diabetes. Diabetes. 54:1763-1769.
27. Vanhecke, D., Leclercq, G., Plum, J., et al. 1995. Characterization of distinct stages during the differentiation of human CD69+CD3+thymocytes and identification of thymic emigrants. J. Immunol. 155: 1862-1872.
28. Smith, S. H., Brown, M. H., Rowe, D., et al. 1986. Functional subsets of human helper-inducer cells defined by a new monoclonal antibody UCHL1. Immunology 58:63-70.
29. Kallas, E. G., Gibbons, D. C., Soucier, H., et al. 1999. Detection of intracellular antigen-specific cytokines in human T cell populations. J. Infect. Dis. 179: 1124-1131.
30. Sato, A., Tsuji, K., Yamamura, M., et al. 1998. Increased type-2 cytokine expression by both CD4+ CD45RO+ T cells and CD8+ CD45RO+ T cells in blood circulation is associated with high serum IgE but not with atopic dermatitis. J. Invest. Dermatol. 111: 1079-1084.

### Example 9

### Reversal of Lymphopenia

In this experiment, 2.5×10⁶ highly purified CD4+ CD44^{v.low} cells (isolated from immunocompetent, untreated donor mice) are injected into recipient mice that do not have an immune system. The lymphoid organs (lymph nodes and spleen) are isolated 3-4 weeks later. Single cell suspensions are made and the cells are incubated with monoclonal antibodies that identify specific immune cell subsets. Mice that are not injected with the cells contain no CD4+ T cells. By this time point after injection of the cells, the lymphoid organs within the host mouse contain CD4+ T cells that express a very low density, an intermediate density (naïve CD4+ T cells) and a high density (memory CD4+ T cells) of CD44, suggesting that this CD4+ CD44^{v.low} cells subset can differentiate to repopulate both the naïve and memory cell subset. Based on the phenotype of the cells, they also give rise to regulatory CD4+ T cells, a cell subset that is critical to maintaining immune balance in the host. In addition, there were more CD4+ CD44^{v.low} cells in the host mouse at this point than the number injected into the mouse, indicating that they also repopulate themselves. Although injection of CD4+ T cells that express either an intermediate or a high density of CD44 also appear to repopulated the mouse to a small extent, they do so very inefficiently. The ability to reverse lymphopenia is important therapeutically in many chronic disease states, including aging. Thus, chronic disease (AIDS, autoimmunity, cancer, failure to thrive syndrome in aging, sepsis) is associated with lymphopenia, and lymphopenia is associated with wasting/muscle atrophy and poor responsiveness to therapy.

### Example 10

### Increase in Insulin Secreting Beta Cell Mass

The protocol for this is that same as that described in Examples 3 and 4 except that the cells were injected into immunoincompetent hosts and not NOD hosts. 4-8 weeks after CD4+ CD44^{v.low} cell injection the pancreas was removed and analyzed by immunohistochemistry for the presence of insulin. Data showed that mice injected with this cell subset, but not other CD4+ T cell subsets, induced a significant increase in the amount of insulin in the pancreas, suggesting that CD4+ CD44^{v.low} cells promote an increase in insulin secretion. The reason a significant increase in insulin secretion was not observed in the pancreas of NOD mice described herein is that the NOD mice are diabetic and their insulin secreting cells are being destroyed by an autoimmune process so the net effect in insulin secretion is negligible. The ability to increase insulin-secreting beta cell mass in the pancreas might be important therapeutically to reverse diabetes in diabetic patients, to promote the growth of islet transplants in islet transplanted patients, to delay the loss of the honeymoon period, and to treat cachexic patients.

### Example 11

### CD4+ CD44int can Differentiate to Become CD4+ CD44^{v.low} Cells

CD4+ regulatory cells, cells that express the regulatory marker, Foxp3, are required to prevent the immune system from causing damage to the host, as it does in autoimmunity and transplant rejection. Purified CD4+ T cells that have an intermediate density of CD44, and that do not contain any Foxp3+cells, are able to differentiate into CD4+ CD44^{v.low} cells if injected into immunodeficient hosts.

What is claimed is:

1. A method of treating, ameliorating, preventing, or delaying the onset of cachexia in a patient comprising:
administering isolated or purified CD4+ T cells to the patient.

2. The method of claim 1, further comprising:
obtaining a cell sample from a mammal;
isolating or purifiying CD4+ T cells from the cell sample; and
expanding the isolated or purified T cells.

3. The method of claim 1, wherein the isolated or purified CD4+ T cells are CD4+ CD44^{v.low}

4. The method of claim 2, wherein the cell sample comprises a blood sample.

5. The method of claim 2, wherein the cell sample comprises a tissue sample.

6. The method of claim 2, wherein the cell sample comprises lymph tissue.

7. The method of claim 2, wherein the mammal is the patient.

8. The method of claim 2, wherein the mammal is not the patient.

9. The method of claim 1, wherein the cachexia is associated with a disease selected from the group consisting of diabetes mellitus, cancer, AIDS, aging, an autoimmune disorder, chronic viral infection, chronic bacterial infection, chronic fungal infection, and end-stage organ failure.

10. The method of claim 9, wherein the disease is diabetes mellitus.

11. The method of claim 10, wherein the diabetes mellitus is Type I diabetes.

12. The method of claim 1, wherein the isolated T cells are isolated using antibodies.

13. The method of claim 12, wherein the antibodies are at least one of anti-CD4 and anti-CD44.

14. The method of claim 1, wherein the isolated T cells are expanded with growth factors.

15. The method of claim 1, wherein the T cells are administered to the patient by one or more of the routes consisting of intravenous, intraperitoneal, intramuscular, subcutaneous, nasal and oral.

16. The method of claim 1, wherein the T cells are administered to the patient by an intramuscular route.

17. The method of claim 1, wherein the patient is human.

18. The method of claim 17, wherein the T cells administered to the human patient comprise between about 10⁸ and about 10¹¹ cells.

19. An isolated T cell population, comprising an isolated population of T cells characterized as CD4+ CD44^{v.low}.

20. The isolated T cell population of claim 19, further in combination with an aqueous vehicle and an additional pharmaceutically acceptable excipient.

21. A method of inhibiting or reversing lymphopenia in a patient comprising:
administering isolated or purified CD4+ T cells to the patient.

22. The method of claim 21, further comprising:
obtaining a cell sample from a mammal;
isolating or purifying CD4+ T cells from the cell sample; and
expanding the isolated or purified T cells.

23. The method of claim 22, wherein the isolated or purified CD4+ T cells are CD4+ CD44^{v.low}.

24. The method of claim 22, further comprising providing to said patient a therapy.

25. A method of treating, ameliorating or preventing diabetes in a patient comprising:
administering isolated CD4+ T cells to the patient.

26. The method of claim 25, further comprising:
obtaining a cell sample from a mammal;
isolating CD4+ T cells from the cell sample; and
expanding the isolated T cells.

27. The method of claim 25, wherein the isolated CD4+ T cells are CD4+ CD44^{v.low}.

28. A method for diagnosing cachexia in a patient, comprising:
identifying a patient at risk for cachexia;
determining a level of CD4+ CD44^{v.low} T cells in a biological sample from said patient; and
assessing whether the amount of CD4+ CD44^{v.low} T cells is at a level which is lower than a predetermined level.

29. A method for diagnosing the onset of a honeymoon period in a patient suffering from Type 1 diabetes, comprising:
identifying a patient with Type 1 diabetes prior to said honeymoon period;
determining a level of CD4+ CD44^{v.low} T cells in a biological sample from said patient; and
assessing whether the amount of CD4+ CD44^{v.low} T cells is at a level which is higher than a predetermined level.

30. A method for diagnosing the loss of a honeymoon period in a patient suffering from Type 1 diabetes, comprising:
identifying a patient with Type 1 diabetes within said honeymoon period;
determining a level of CD4+ CD44^{v.low} T cells in a biological sample from said patient; and
assessing whether the amount of CD4+ CD44^{v.low} T cells is at a level which is lower than a predetermined level.

31. A method for monitoring the progress of a cachexia therapy in a patient comprising:
identifying a patient with cachexia;
providing said subject a cachexia therapy;
determining a level of CD4+ CD44^{v.low} T cells in a biological sample in said patient, before a treatment with said cachexia therapy and during or after a period of said treatment.

32. A method for determining the response to a cachexia therapy in a patient comprising:
identifying a patient with a cachexia;
providing said patient a cachexia therapy; and
determining a level of CD4+ CD44^{v.low} T cells in a biological sample in said patient, before a treatment with said cachexia therapy and during or after a period of said treatment.

33. A method for promoting the responsiveness to a therapy for a disorder:
identifying a patient with the disorder;
administering isolated or purified CD4+ T cells to the patient;

34. The method of claim 33, further comprising:
obtaining a cell sample from a mammal;
isolating or purifiying CD4+ T cells from the cell sample; and
expanding the isolated or purified T cells.

35. The method of claim 33, wherein the isolated or purified CD4+ T cells are CD4+ CD44^{v.low}.

36. The method of claim 35, further comprising providing to said patient said therapy for said disorder.

37. The method of claim 33, wherein said disorder is cachexia.

38. The method of claim 33, wherein said disorder is Type I diabetes.

39. A method of identifying a patient likely to be responsive to a therapy for a disorder comprising:
identifying a patient with said disorder;
determining a level of CD4+ CD44^{v.low} T cells in a biological sample from said patient; and
assessing whether the amount of CD4+ CD44^{v.low} T cells is at a level which is greater than a predetermined level.

40. The method of claim 39, wherein said disorder is cachexia.

41. The method of claim 39, wherein said disorder is Type 1 diabetes.

42. A method for promoting the onset of the honeymoon period in Type 1 diabetes, comprising:
administering isolated or purified CD4+ T cells to the patient.

43. The method of claim 42, further comprising:
obtaining a cell sample from a mammal;
isolating or purifiying CD4+ T cells from the cell sample; and
expanding the isolated or purified T cells.

44. The method of claim 42, wherein the isolated or purified CD4+ T cells are CD4+ CD44^{v.low}.

45. A method for delaying the loss of the honeymoon period in Type 1 diabetes, comprising:
administering isolated or purified CD4+ T cells to the patient.

46. The method of claim 45, further comprising:
obtaining a cell sample from a mammal;
isolating or purifiying CD4+ T cells from the cell sample; and
expanding the isolated or purified T cells.

47. The method of claim 45, wherein the isolated or purified CD4+ T cells are CD4+ CD44^{v.low}.

48. A method of treating, ameliorating or preventing diabetes in a patient comprising:
isolating or purifying pancreatic islets from said patient or other donor;
growing said islets in culture in the presence of CD4+ T cells;
and transplanting said islets into said patient.

49. The method of claim 48, wherein the CD4+ T cells are CD4+ CD44^{v.low} cells.


SEQUENCE LISTING (6 sequences)

SEQ ID NO: 1 - DNA, 25 bp, Artificial Sequence
  B-actin forward primer
tggaatcctgtggcatccatgaaac

SEQ ID NO: 2 - DNA, 24 bp, Artificial Sequence
  B-actin reverse primer
taaaacgcagctcagtaacagtcc

SEQ ID NO: 3 - DNA, 20 bp, Artificial Sequence
  MuRF1 forward primer
gtccatgtctggaggtcgtt

SEQ ID NO: 4 - DNA, 20 bp, Artificial Sequence
  MuRF1 reverse primer
gtggacttttccagctgctc

SEQ ID NO: 5 - DNA, 20 bp, Artificial Sequence
  MAFbx forward primer
gaacatcatgcagaggctga

SEQ ID NO: 6 - DNA, 20 bp, Artificial Sequence
  MAFbx reverse primer
cttcttggcctgctgaaaac